(19) United States
(12) Patent Application Publication
(10) Pub. No.: US 2024/0209071 A1
(43) Pub. Date: Jun. 27, 2024

(54) DOSAGE AND ADMINISTRATION OF ANTI-C5 ANTIBODIES FOR TREATING HEMATOPOIETIC STEM CELL TRANSPLANT-ASSOCIATED THROMBOTIC MICROANGIOPATHY (HSCT-TMA)

(71) Applicant: Alexion Pharmaceuticals, Inc., Boston, MA (US)

(72) Inventors: Stephan Ortiz, Wellesley, MA (US); Jonathan Monteleone, Raleigh, NC (US)

(73) Assignee: Alexion Pharmaceuticals, Inc., Boston, MA (US)

(21) Appl. No.: 18/018,936

(22) PCT Filed: Aug. 12, 2021
(86) PCT No.: PCT/US2021/045823

§ 371 Date: Jan. 31, 2023

Related U.S. Application Data
(60) Provisional application No. 63/065,107, filed on Aug. 13, 2020.

(51) Int. Cl. C07K 16/18; A61K 9/00; A61K 39/395; A61K 45/06; A61P 7/02

(52) U.S. Cl. CPC C07K 16/18; A61K 9/0019; A61K 39/3955; A61K 45/06; A61P 7/02; A61K 2039/505; C07K 2317/52; C07K 2317/565; C07K 2317/76; C07K 2317/92

(57) ABSTRACT

Provided are methods for clinical treatment of hematopoietic stem cell transplant-associated thrombotic microangiopathy (HSCT-TMA), e.g., TMA after HSCT, in human patients using an anti-C5 antibody, or antigen binding fragment thereof.

### CROSS REFERENCE TO RELATED APPLICATIONS

This application claims the benefit of U.S. Provisional Application No. 63/065,107, filed Aug. 13, 2020, the contents of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Aug. 10, 2021 is named 0630_WO_SL.txt and is 58,856 bytes in size.

### BACKGROUND

Thrombotic microangiopathy (TMA) is a post-transplant complication of hematopoietic stem cell transplant (HSCT) that may affect 10% to 35% of HSCT recipients (Dvorak et al., Front Pediatr. 2019; 7:133); Jodele et al., Blood Rev. 2015; 29(3):191-204; Seaby et al., Pediatr Nephrol. 2018; 33(9):1489-1500). It presents via endothelial injury and affects the kidney and other organs. It is estimated that 30% of patients with HSCT TMA will present with severe disease (Rosenthal et al., J. Blood Med. 2016; 7:181-186), and in these cases, patients with HSCT-TMA develop systemic vascular injury manifesting in kidney damage, serositis, pulmonary hypertension, and multisystem organ failure. Survival of patients with HSCT-TMA at 1 year has been reported to range from 18% to 40% (Wanchoo et al., Am. J Kidney Dis. 2018; 72(6):857-865). Severe HSCT-TMA is associated with long-term morbidity and mortality rates of approximately 80%. Studies have shown that the large majority of patients die within 6 months (Cho et al., Bone Marrow Transplant. 2008; 41(9):813-820; Cho et al., Transplantation. 2010; 90(8):918-926; Oran, 2007). Another study showed 9% overall survival of patients with severe HSCT-TMA who did not receive TMA targeted therapy, with all mortality occurring within 10 months of the TMA diagnosis (Jodele, et al., Blood. 2014b; 124(4):645-653).

In pediatric patients, HSCT-TMA typically occurs early post allogeneic HSCT, with a median diagnosis at 35 to 47 days post-HSCT, and 88% to 92% occurring before Day +100. However, cases have been reported up to 2 years post-HSCT. Autologous recipients may develop HSCT TMA even earlier, with a median of 18 days post-HSCT (Dvorak et al., Front Pediatr. 2019; 7:133).

Endothelial injury is fundamental to the pathogenesis of HSCT-TMA, with dysregulated complement activation likely to be a consequence of the endothelial damage. Risk factors associated with HSCT-TMA development that also initiate endothelial damage include calcineurin inhibitors (CNIs), infections, and conditioning regimens (high dose chemotherapy or total body irradiation) (Khosla, et al., Bone Marrow Transplant. 2018; 53(2):129-137; Masias, et al., Blood. 2017; 129(21):2857-2863).

Currently, there are no approved therapies for the treatment of HSCT-TMA. Primary intervention for HSCT-TMA involves withdrawal of the offending agent (e.g., CNIs) and/or treatment of any trigger conditions (e.g., treatment for infections) (Dvorak, et al., Front Pediatr. 2019; 7:133); Seaby et al., Pediatr. Nephrol. 2018; 33(9):1489-1500). In some patients, withdrawal of the offending agent and/or treatment of any associated trigger condition does not reverse the HSCT-TMA. Accordingly, it is an object of the present disclosure to provide improved methods for treating patients with HSCT-TMA.

### SUMMARY

Provided herein are compositions and methods for treating hematopoietic stem cell transplant-associated thrombotic microangiopathy (HSCT-TMA), e.g., TMA after HSCT, in a human patient, comprising administering to the patient an anti-C5 antibody or antigen binding fragment thereof, wherein the anti-C5 antibody or antigen binding fragment thereof is administered (or is for administration) according to a particular clinical dosage regimen (e.g., at a particular dose amount and according to a specific dosing schedule).

An exemplary anti-C5 antibody is ravulizumab (ULTOMIRIS®) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of ravulizumab. Accordingly, in one embodiment, the antibody comprises the CDR1, CDR2 and CDR3 domains of the heavy chain variable (VH) region of ravulizumab having the sequence shown in SEQ ID NO:12, and the CDR1, CDR2 and CDR3 domains of the light chain variable (VL) region of ravulizumab having the sequence shown in SEQ ID NO:8. In another embodiment, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively. In another embodiment, the antibody comprises VH and VL regions having the amino acid sequences set forth in SEQ ID NO:12 and SEQ ID NO:8, respectively. In another embodiment, the antibody comprises a heavy chain constant region as set forth in SEQ ID NO:13.

In another embodiment, the antibody comprises a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention.

In another embodiment, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention.

In another embodiment, the anti-C5 antibody comprises the heavy and light chain CDRs or variable regions of the BNJ421 antibody (described in WO2015134894 and U.S. Pat. No. 9,079,949). In another embodiment, the anti-C5 antibody comprises the heavy and light chain CDRs or variable regions of the 7086 antibody (see U.S. Pat. Nos. 8,241,628 and 8,883,158). In another embodiment, the anti-C5 antibody comprises the heavy and light chain CDRs or variable regions of the 8110 antibody (see U.S. Pat. Nos. 8,241,628 and 8,883,158). In another embodiment, the anti-C5 antibody comprises the heavy and light chain CDRs or variable regions of the 305LO5 antibody (see U.S. Pat. No. 9,765,135). In another embodiment, the anti-C5 antibody comprises the heavy and light chain CDRs or variable regions of the SKY59 antibody. In another embodiment, the anti-C5 antibody comprises the heavy and light chain CDRs or variable regions of the REGN3918 antibody.

In another embodiment, the antibody competes for binding with, and/or binds to the same epitope on C5 as any of the above-mentioned antibodies. In another embodiment, the antibody has at least about 90% variable region amino acid sequence identity to any of the above-mentioned antibodies (e.g., at least about 90%, 95% or 99% variable region identity with SEQ ID NO:12 or SEQ ID NO:8).

In another embodiment, the antibody binds to human C5 at pH 7.4 and 25° C. with an affinity dissociation constant (K_{D}) that is in the range 0.1 nM≤K_{D}≤1 nM. In another embodiment, the antibody binds to human C5 at pH 7.4 and 25° C. with an affinity dissociation constant (K_{D}) of about 0.5 nM. In another embodiment, the antibody binds to human C5 at pH 6.0 and 25° C. with a K_{D}≥10 nM. In another embodiment, the antibody binds to human C5 at pH 6.0 and 25° C. with a K_{D} of about 22 nM. In yet another embodiment, the [(K_{D} of the antibody or antigen-binding fragment thereof for human C5 at pH 6.0 and at 25° C.)/(K_{D} of the antibody or antigen-binding fragment thereof for human C5 at pH 7.4 and at 25° C.)] of the antibody is greater than 25.

In one embodiment, the dose of the anti-C5 antibody, or antigen binding fragment thereof, is based on the weight of the patient. In one embodiment, for example, 300 mg or 600 mg of the anti-C5 antibody, or antigen binding fragment thereof, is administered to a patient weighing ≥5 to <10 kg. In another embodiment, 300 mg or 600 mg of the anti-C5 antibody, or antigen binding fragment thereof, is administered to a patient weighing ≥10 to <20 kg. In another embodiment, 300 mg, 900 mg, or 2100 mg of the anti-C5 antibody, or antigen binding fragment thereof, is administered to a patient weighing ≥20 to <30 kg. In another embodiment, 300 mg, 1200 mg, or 2700 mg of the anti-C5 antibody, or antigen binding fragment thereof, is administered to a patient weighing ≥30 to <40 kg. In another embodiment, 600 mg, 2400 mg, or 3000 mg of the anti-C5 antibody, or antigen binding fragment thereof, is administered to a patient weighing ≥40 to <60 kg. In another embodiment, 900 mg, 2700 mg, or 3300 mg of the anti-C5 antibody, or antigen binding fragment thereof, is administered to a patient weighing ≥60 to <100 kg. In another embodiment, 900 mg, 3000 mg, or 3600 mg of the anti-C5 antibody, or antigen binding fragment thereof, is administered to a patient weighing ≥100 kg. In certain embodiments, dosage regimens are adjusted to provide the optimum desired response (e.g., an effective response).

In another embodiment, the anti-C5 antibody, or antigen binding fragment thereof, is administered for one or more administration cycles. In one embodiment, the treatment (e.g., administration cycle) is 26 weeks. In one embodiment, the anti-C5 antibody, or antigen binding fragment thereof, is administered once on Day 1 (e.g., of the administration cycle), once on Day 5 (e.g., of the administration cycle), once on Day 10 (e.g., of the administration cycle). In another embodiment, the anti-C5 antibody, or antigen binding fragment thereof, is administered is administered once on Day 1 (e.g., of the administration cycle), once on Day 5 (e.g., of the administration cycle), once on Day 10 (e.g., of the administration cycle) and every eight four or eight weeks thereafter starting at Day 15. In another embodiment, the anti-C5 antibody, or antigen binding fragment thereof, is administered every four or eight weeks after treatment (e.g., an administration cycle) for an extension period up to two years (e.g., at a dose of 300 mg, 600 mg, 900 mg, 1200 mg, 2100 mg, 2400 mg, 2700 mg, 3000 mg, 3300 mg or 3600 mg).

In another embodiment, a method of treating a human patient with HSCT-TMA is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In another embodiment, a method of treating a human patient with HSCT-TMA is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg; and wherein the treatment results in a reduction in LDH levels to within normal levels or to within 50% below what is considered the ULN level (e.g., within 105-333 IU/L (international units per liter), a serum trough concentration of the anti-C5 antibody, or antigen binding fragment thereof, of at least 175 μg/mL or greater, and/or a free C5 concentration of 0.5 μg/mL or less (e.g., 0.4 μg/mL, 0.3 μg/mL, 0.2 μg/mL, or 0.1 μg/mL or less).

In another embodiment, a method of treating a human patient with HSCT-TMA is provided, the method comprising administering to the patient (e.g., during an administration cycle) an effective amount of an anti-C5 antibody, or antigen binding fragment thereof, comprising CDR1, CDR2 and CDR3 heavy chain sequences as set forth in SEQ ID NOs:19, 18 and 3, respectively, CDR1, CDR2 and CDR3 light chain sequences as set forth in SEQ ID NOs:4, 5 and 6, respectively, and a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention, wherein the anti-C5 antibody, or antigen binding fragment thereof, is administered:
(a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In another embodiment, the anti-C5 antibody or antigen binding fragment thereof is administered to a patient weighing ≥5 to <10 kg:
(a) once on Day 1 at a dose of 600 mg;
(b) once on Day 5 at a dose of 300 mg;
(c) once on Day 10 at a dose of 300 mg; and
(d) on Day 15 and every four weeks thereafter at a dose of 300 mg.

In another embodiment, the anti-C5 antibody is administered to a patient weighing ≥10 to <20 kg:
(a) once on Day 1 at a dose of 600 mg;
(b) once on Day 5 at a dose of 300 mg;
(c) once on Day 10 at a dose of 300 mg; and
(d) on Day 15 and every four weeks thereafter at a dose of 600 mg.

In another embodiment, the anti-C5 antibody is administered to a patient weighing ≥20 to <30 kg:
(a) once on Day 1 at a dose of 900 mg;
(b) once on Day 5 at a dose of 300 mg;
(c) once on Day 10 at a dose of 300 mg; and
(d) on Day 15 and every eight weeks thereafter at a dose of 2100 mg.

In another embodiment, the anti-C5 antibody is administered to a patient weighing ≥30 to <40 kg:
(a) once on Day 1 at a dose of 1200 mg;
(b) once on Day 5 at a dose of 300 mg;
(c) once on Day 10 at a dose of 300 mg; and
(d) on Day 15 and every eight weeks thereafter at a dose of 2700 mg.

In another embodiment, the anti-C5 antibody is administered to a patient weighing ≥40 to <60 kg:
(a) once on Day 1 at a dose of 2400 mg;
(b) once on Day 5 at a dose of 600 mg;
(c) once on Day 10 at a dose of 600 mg; and
(d) on Day 15 and every eight weeks thereafter at a dose of 3000 mg.

In another embodiment, the anti-C5 antibody is administered to a patient weighing ≥60 to <100 kg:
(a) once on Day 1 at a dose of 2700 mg;
(b) once on Day 5 at a dose of 900 mg;
(c) once on Day 10 at a dose of 900 mg; and
(d) on Day 15 and every eight weeks thereafter at a dose of 3300 mg.

In another embodiment, the anti-C5 antibody is administered to a patient weighing ≥100 kg:
(a) once on Day 1 at a dose of 3000 mg;
(b) once on Day 5 at a dose of 900 mg;
(c) once on Day 10 at a dose of 900 mg; and
(d) on Day 15 and every eight weeks thereafter at a dose of 3600 mg.

In one embodiment, the methods of treatment disclosed herein further comprise administering best supportive care (BSC) measures. BSC measures include, but are not limited to, transfusion support, corticosteroids, dialysis, and antihypertensive medications.

In one embodiment, the patient has not previously been treated with eculizumab. In another embodiment, the patient has previously been treated with eculizumab. In another embodiment, the patient has previously been treated with eculizumab and Day 1 (e.g., of the administration cycle) is two weeks or more from the patient's last dose of eculizumab.

In another aspect, the treatment regimens described are sufficient to maintain particular serum trough concentrations of the anti-C5 antibody or antigen binding fragment thereof. In one embodiment, for example, the treatment regimen maintains a serum trough concentration of the anti-C5 antibody or antigen binding fragment thereof of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395 or 400 μg/mL or greater. In one embodiment, the treatment regimen maintains a serum trough concentration of the anti-C5 antibody or antigen binding fragment thereof of 100 μg/mL or greater, 150 μg/mL or greater, 200 μg/mL or greater, 250 μg/mL or greater, or 300 μg/mL or greater. In another embodiment, the treatment maintains a serum trough concentration of the anti-C5 antibody or antigen binding fragment thereof of between 100 μg/mL and 200 μg/mL. In another embodiment, the treatment maintains a serum trough concentration of the anti-C5 antibody or antigen binding fragment thereof of about 175 μg/mL.

In another embodiment, to obtain an effective response, the anti-C5 antibody is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg or 260 μg of antibody per milliliter of the patient's blood. In another embodiment, the anti-C5 antibody is administered to the patient in an amount and with a frequency to maintain between 50 μg and 250 μg of antibody per milliliter of the patient's blood. In another embodiment, the anti-C5 antibody is administered to the patient in an amount and with a frequency to maintain between 100 μg and 200 μg of antibody per milliliter of the patient's blood. In another embodiment, the anti-C5 antibody is administered to the patient in an amount and with a frequency to maintain about 175 μg of antibody per milliliter of the patient's blood.

In another embodiment, to obtain an effective response, the anti-C5 antibody is administered to the patient in an amount and with a frequency to maintain a minimum free C5 concentration. In one embodiment, for example, the anti-C5 antibody is administered to the patient in an amount and with a frequency to maintain a free C5 concentration of 0.5 μg/mL or less (e.g., 0.4 μg/mL, 0.3 μg/mL, 0.2 μg/mL, or 0.1 μg/mL or less).

The anti-C5 antibodies, or antigen binding fragments thereof, can be administered to a patient by any suitable means. In one embodiment, the antibodies are formulated for intravenous administration.

The efficacy of the treatment methods provided herein can be assessed using any suitable means. In one embodiment, for a HSCT-TMA patient, the treatment produces at least one therapeutic effect selected from the group consisting of: a reduction or cessation in microangiopathic hemolytic anemia, thrombocytopenia, endothelial injury, kidney damage, kidney failure, serositis, pulmonary hypertension, and multisystem organ failure compared to baseline.

In another embodiment, the treatment results in (a) platelet count ≥50,000/mm³ without transfusion support during the prior 7 days, (b) LDH<1.5×ULN, and (c) absence of schistocytes (if there were schistocytes present at baseline).

In another embodiment, the treatment results in (a) platelet count ≥50,000/mm³ without transfusion support during the prior 7 days, (b) LDH<1.5×ULN, (c) absence of schistocytes (if there were schistocytes present at baseline) and (d) at least 50% reduction of proteinuria from baseline. In one embodiment, proteinuria is as protein/creatinine ratio ≥0.5 mg/mg.

In another embodiment, the treatment results in a favorable hematological response.

In another embodiment, the treatment results in normalization of LDH, resolution of need for red cell and platelet transfusions, and disappearance of schistocytes.

In another embodiment, the treatment results in the patient's ability to maintain hemoglobin ≥8 g/dL without transfusion support. In another embodiment, the treatment results in hemoglobin ≥8 g/dL without transfusion support.

In another embodiment, the treatment results in a decrease in LDH, an increase in platelets, and/or an increase in hemoglobin compared to baseline.

In another embodiment, the treatment results in normal levels of serum creatinine compared to baseline.

In another embodiment, the treatment results in an improvement in TMA-associated organ dysfunction in the renal, cardiovascular, pulmonary, CNS, and/or GI systems compared to baseline.

In another embodiment, the treatment results in terminal complement inhibition.

In another embodiment, the treatment produces a reduction in adverse events.

In another embodiment, the treatment produces a shift toward normal levels of biomarkers associated with vascular inflammation (e.g., shed tumor necrosis factor receptor 1 [TNF-R1]), endothelial damage and/or activation (e.g., thrombomodulin and shed vascular cell adhesion molecule 1 [VCAM-1]), renal injury (e.g., Cystatin C), and/or complement proteins and complement activation pathway products.

In another embodiment, the treatment produces a change from baseline in quality of life as assessed via a Quality of Life Assessment (e.g., a Quality of Life Inventory (PedsQL) Scale or an EQ-5D-5L questionnaire). Exemplary are Quality of Life Assessment are set forth in FIGS. 4-10 and 15.

In another embodiment, the treatment results in a reduction of hemolysis as assessed by lactate dehydrogenase (LDH) levels compared to baseline. In one embodiment, patients treated according to the disclosed methods experience reductions in LDH levels to near normal levels or to within 10%, or within 20% above what is considered the normal level (e.g., within 105-333 IU/L (international units per liter). In another embodiment, the patient's LDH levels are normalized throughout maintenance period of treatment. In another embodiment, the treated patient's LDH levels are normalized at least at least 95% of the time while on the maintenance period of treatment. In another embodiment, the treated patient's LDH levels are normalized at least at least 90%, 85% or 80% of the time while on the maintenance period of treatment. In one embodiment, the patient's LDH levels are ≥1.5 fold above the upper limit of normal (LDH≥1.5×ULN) prior to initiating treatment.

In one embodiment, patients treated according to the disclosed methods experience reductions in LDH levels to within normal levels or to within 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% 40%, 4%1%, 42%, 43%, 44%, 45%, 46%, 47%, 48%, 49% or within 50% below what is considered the ULN level (e.g., within 105-333 IU/L (international units per liter). In one embodiment, the patient's LDH levels are ≥1.5 fold above the ULN (LDH≥1.5×ULN) prior to initiating treatment.

In one embodiment, patients treated according to the disclosed methods experience an LDH percent change compared to baseline of 10%, 11%, 12%, 13%, 14%, 15%, 16%, 17%, 1⁸%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53% 54%, 55%, 56%, 57%, 58%, 59%, or 60%.

In one embodiment, patients treated according to the disclosed methods maintain a serum trough concentration of the anti-C5 antibody, or antigen binding fragment thereof, of at least 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395 or 400 μg/mL or greater. In one embodiment, patients treated according to the disclosed methods maintain a serum trough concentration of the anti-C5 antibody, or antigen binding fragment thereof, of at least 175 μg/mL or greater.

In one embodiment, patients treated according to the disclosed methods have a free C5 concentration of 0.5 μg/mL or less (e.g., 0.4 μg/mL, 0.3 μg/mL, 0.2 μg/mL, or 0.1 μg/mL or less).

In another aspect, an anti-C5 antibody or antigen binding fragment thereof is provided, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8, for administration:
(a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In one embodiment, the antibody is determined to be safe, tolerable and sufficiently non-immunogenic after multiple IV doses for use in HSCT-TMA patients.

Further provided are kits that include a pharmaceutical composition containing an anti-C5 antibody or antigen binding fragment thereof, such as ravulizumab, and a pharmaceutically acceptable carrier, in a therapeutically effective amount adapted for use in the methods described herein. In one embodiment, the kit comprises: (a) a dose of an anti-C5 antibody or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and (b) instructions for using the anti-C5 antibody or antigen binding fragment thereof in the methods described herein.

In one embodiment, 300 mg or 600 mg of the anti-C5 antibody, or antigen binding fragment thereof, is administered to a patient weighing ≥5 to <10 kg. In another embodiment, 300 mg or 600 mg of the anti-C5 antibody, or antigen binding fragment thereof, is administered to a patient weighing ≥10 to <20 kg. In another embodiment, 300 mg, 900 mg, or 2100 mg of the anti-C5 antibody, or antigen binding fragment thereof, is administered to a patient weighing ≥20 to <30 kg. In another embodiment, 300 mg, 1200 mg, or 2700 mg of the anti-C5 antibody, or antigen binding fragment thereof, is administered to a patient weighing ≥30 to <40 kg. In another embodiment, 600 mg, 2400 mg, or 3000 mg of the anti-C5 antibody, or antigen binding fragment thereof, is administered to a patient weighing ≥40 to <60 kg. In another embodiment, 900 mg, 2700 mg, or 3300 mg of the anti-C5 antibody, or antigen binding fragment thereof, is administered to a patient weighing ≥60 to <100 kg. In another embodiment, 900 mg, 3000 mg, or 3600 mg of the anti-C5 antibody, or antigen binding fragment thereof, is administered to a patient weighing ≥100 kg.

In some embodiments, the disclosure relates to a composition, e.g., pharmaceutical composition or a medicament, comprising an effective amount of an anti-C5 antibody or an antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, 5 and 6, respectively, for use in the treatment of hematopoietic stem cell transplant-associated thrombotic microangiopathy (HSCT-TMA), e.g., TMA after HSCT, in a human patient. Specifically, provided herein are compositions comprising effective amounts of ravulizumab (ULTOMIRIS®) or the antigen-binding fragment thereof, for treatment of HSCT-TMA, e.g., TMA after HSCT, in a human patient. In some embodiments, the effective amount comprises use of the above dosages and scheduling of the anti-C5 antibody, e.g., ravulizumab.

In some embodiments, the disclosure relates to use of an effective amount of an anti-C5 antibody or antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, 5 and 6, respectively, in the manufacture of a composition, e.g., pharmaceutical composition or a medicament, for treating hematopoietic stem cell transplant-associated thrombotic microangiopathy (HSCT-TMA), e.g., TMA after HSCT, in a human patient. Specifically, provided herein are use of an effective amount of ravulizumab (ULTOMIRIS®) or the antigen-binding fragment thereof, in the manufacture of a composition, e.g., pharmaceutical composition or a medicament, for treating HSCT-TMA, e.g., TMA after HSCT, in a human patient. In some embodiments, the effective amount comprises use of the above dosages and scheduling of the anti-C5 antibody, e.g., ravulizumab.

### BRIEF DESCRIPTION OF THE DRAWINGS

FIG. 1 is a schematic depicting the overall design for the pediatric phase 3 trial.

FIG. 2 sets forth the schedule of study visits and assessments through completion of the 26-week Treatment Period of the pediatric phase 3 clinical trial.

FIG. 3 sets forth the schedule of study visits and assessments for the Follow-up Period of the pediatric phase 3 clinical trial.

FIG. 4 is a sample Pediatric Quality of Life Inventory (PedsQL) 4.0 Generic Core Scale for Toddlers (ages 2-4).

FIG. 5 is a sample PedsQL 4.0 Generic Core Scale for a Young Child (ages 5-7).

FIG. 6 is a second sample PedsQL 4.0 Generic Core Scale for a Young Child (ages 5-7).

FIG. 7 is a sample PedsQL 4.0 Generic Core Scale for a Young Child (ages 8-12).

FIG. 8 is a second sample PedsQL 4.0 Generic Core Scale for a Young Child (ages 8-12).

FIG. 9 is a sample PedsQL 4.0 Generic Core Scale for a Young Child (ages 13-18).

FIG. 10 is a second sample PedsQL 4.0 Generic Core Scale for a Young Child (ages 13-18).

FIG. 11 is a schematic depicting the overall design for the adult phase 3 trial.

FIG. 12 sets forth the schedule of activities for the screening and treatment Period (Stage 1 and Stage 2 Participants) of the adult phase 3 clinical trial.

FIG. 13 sets forth the schedule of activities for the treatment period (participants who receive ravulizumab rescue therapy) of the adult phase 3 clinical trial.

FIG. 14 sets forth the schedule of activities for the follow-up period of the adult phase 3 clinical trial.

FIG. 15 is a sample Qol for an adult.

### DETAILED DESCRIPTION

### I. Definitions

As used herein, the term “subject” or “patient” is a human patient (e.g., a patient having hematopoietic stem cell transplant-associated thrombotic microangiopathy (HSCT-TMA)).

As used herein, the term “pediatric” patient is a human patient that has been classified by a physician or caretaker as belonging to a non-adult category and can include, e.g., newborn (both preterm and of term), infants, children, and adolescents. Typically, pediatric patients are patients under 18 years of age (<18 years of age).

As used herein, the term “adult” patient is a human patient that has been classified by a physician or caretaker as such, e.g., one who is not a newborn, infant, child or adolescent, e.g., based on age, developmental status, physiological features, etc. Typically, adult patients are patients who are 18 years of age or older (≥18 years of age).

HSCT-TMA is a multifactorial disorder caused by systemic vascular endothelial injury that can be triggered by several mechanisms during the transplant process. It presents via endothelial injury and affects the kidney and other organs. It is estimated that 30% of patients with HSCT TMA will present with severe disease (Rosenthal et al., J. Blood Med. 2016; 7:181-186), and in these cases, patients with HSCT-TMA develop systemic vascular injury manifesting in kidney damage, serositis, pulmonary hypertension, and multisystem organ failure.

As used herein, “effective treatment” refers to treatment producing a beneficial effect, e.g., amelioration of at least one symptom of a disease or disorder. A beneficial effect can take the form of an improvement over baseline, e.g., an improvement over a measurement or observation made prior to initiation of therapy according to the method. Effective treatment may refer to alleviation of at least one symptom of HSCT-TMA (e.g., microangiopathic hemolytic anemia, thrombocytopenia, endothelial injury, kidney damage, kidney failure, serositis, pulmonary hypertension, and multisystem organ failure).

The term “effective amount” refers to an amount of an agent that provides the desired biological, therapeutic and/or prophylactic result. That result can be reduction, amelioration, palliation, lessening, delaying and/or alleviation of one or more of the signs, symptoms or causes of a disease, or any other desired alteration of a biological system. In one example, an “effective amount” is the amount of anti-C5 antibody, or antigen binding fragment thereof, clinically proven to alleviate at least one symptom of HSCT-TMA (e.g., microangiopathic hemolytic anemia, thrombocytopenia, endothelial injury, kidney damage, kidney failure, serositis, pulmonary hypertension, and multisystem organ failure). An effective amount can be administered in one or more administrations.

As used herein, the term “loading dose” refers to the first dose administered (e.g., during an administration cycle).

As used herein, the terms “maintenance” and “maintenance phase” are used interchangeably and refer to the second phase of treatment. In certain embodiments, treatment is continued as long as clinical benefit is observed or until unmanageable toxicity or disease progression occurs.

As used herein, the term “serum trough level” refers to the lowest level that the agent (e.g., the anti-C5 antibody, or antigen binding fragment thereof) or medicine is present in the serum. In contrast, a “peak serum level,” refers to the highest level of the agent in the serum. The “average serum level,” refers to the mean level of the agent in the serum over time.

The term “antibody” describes a polypeptide comprising at least one antibody-derived antigen binding site (e.g., VH/VL region or Fv, or CDR). Antibodies include known forms of antibodies, e.g., the antibody can be a human antibody, a humanized antibody, a bispecific antibody or a chimeric antibody. The antibody also can be a Fab, Fab′2, ScFv, SMIP, Affibody®, nanobody or a single-domain antibody. The antibody also can be of any of the following isotypes: IgG1, IgG2, IgG3, IgG4, IgM, IgA1, IgA2, IgAsec, IgD, IgE or combinations thereof. The antibody can be a naturally occurring antibody or an antibody that has been altered by a protein engineering technique (e.g., by mutation, deletion, substitution, conjugation to a non-antibody moiety). An antibody can include, for example, one or more variant amino acids (compared to a naturally occurring antibody) that change a property (e.g., a functional property) of the antibody. Numerous such alterations are known in the art that affect, e.g., half-life, effector function, and/or immune responses to the antibody in a patient. The term antibody also includes artificial or engineered polypeptide constructs that comprise at least one antibody-derived antigen binding site.

### II. Anti-C5 Antibodies

Anti-C5 antibodies described herein bind to complement component C5 (e.g., human C5) and inhibit the cleavage of C5 into fragments C5a and C5b. As described above, such antibodies also have, for example, improved pharmacokinetic properties relative to other anti-C5 antibodies (e.g., eculizumab) used for therapeutic purposes.

Anti-C5 antibodies (or VH/VL domains derived therefrom) suitable for use in the methods described herein can be generated using methods known in the art. Alternatively, art recognized anti-C5 antibodies can be used. Antibodies that compete for binding to C5 with any of these art recognized antibodies or antibodies described herein can also be used.

An exemplary anti-C5 antibody is ravulizumab comprising heavy and light chains having the sequences shown in SEQ ID NOs:14 and 11, respectively, or antigen binding fragments and variants thereof. Ravulizumab (also known as ULTOMIRIS®, BNJ441 and ALXN1210) is described in WO2015134894 and U.S. Pat. No. 9,079,949, the entire teachings of which are hereby incorporated by reference. The terms ravulizumab, BNJ441, and ALXN1210 may be used interchangeably throughout this document, but all refer to the same antibody. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of ravulizumab. Accordingly, in one embodiment, the antibody comprises the CDR1, CDR2 and CDR3 domains of the VH region of ravulizumab having the sequence set forth in SEQ ID NO:12, and the CDR1, CDR2 and CDR3 domains of the VL region of ravulizumab having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively. In another embodiment, the antibody comprises VH and VL regions having the amino acid sequences set forth in SEQ ID NO: 12 and SEQ ID NO:8, respectively.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in WO2015134894 and U.S. Pat. No. 9,079,949, the entire teachings of which are hereby incorporated by reference.

In other embodiments, the antibody comprises the heavy and light chain CDRs or variable regions of BNJ421. Accordingly, in one embodiment, the antibody comprises the CDR1, CDR2 and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively. In another embodiment, the antibody comprises VH and VL regions having the amino acid sequences set forth in SEQ ID NO: 12 and SEQ ID NO:8, respectively.

The exact boundaries of CDRs are defined differently according to different methods. In some embodiments, the positions of the CDRs or framework regions within a light or heavy chain variable domain are as defined by Kabat et al. [(1991) “Sequences of Proteins of Immunological Interest.” NIH Publication No. 91-3242, U.S. Department of Health and Human Services, Bethesda, MD]. In such cases, the CDRs can be referred to as “Kabat CDRs” (e.g., “Kabat LCDR2” or “Kabat HCDR1”). In some embodiments, the positions of the CDRs of a light or heavy chain variable region are as defined by Chothia et al. (Nature, 342:877-83, 1989). Accordingly, these regions can be referred to as “Chothia CDRs” (e.g., “Chothia LCDR2” or “Chothia HCDR3”). In some embodiments, the positions of the CDRs of the light and heavy chain variable regions can be defined by a Kabat-Chothia combined definition. In such embodiments, these regions can be referred to as “combined Kabat-Chothia CDRs.” Thomas, C. et al. (Mol. Immunol., 33:1389-401, 1996) exemplifies the identification of CDR boundaries according to Kabat and Chothia numbering schemes.

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody, or antigen binding fragment thereof, comprises heavy chain CDR1, CDR2 and CDR3 domains having the sequences set forth in SEQ ID NOs:21, 22 and 23, respectively, and light chain CDR1, CDR2 and CDR3 domains having the sequences set forth in SEQ ID NOs:24, 25 and 26, respectively. In another embodiment, the antibody, or antigen binding fragment thereof, comprises the VH region of the 7086 antibody having the sequence set forth in SEQ ID NO:27, and the VL region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiment, the antibody comprises the heavy and light chain CDRs or variable regions of the 8110 antibody. In another embodiment, the antibody, or antigen binding fragment thereof, comprises heavy chain CDR1, CDR2 and CDR3 domains having the sequences set forth in SEQ ID NOs:29, 30 and 31, respectively, and light chain CDR1, CDR2 and CDR3 domains having the sequences set forth in SEQ ID NOs:32, 33 and 34, respectively. In another embodiment, the antibody comprises the VH region of the 8110 antibody having the sequence set forth in SEQ ID NO:35, and the VL region of the 8110 antibody having the sequence set forth in SEQ ID NO:36.

Another exemplary anti-C5 antibody is the 305LO5 antibody described in U.S. Pat. No. 9,765,135. In one embodiment, the antibody comprises the heavy and light chain CDRs or variable regions of the 305LO5 antibody. In another embodiment, the antibody, or antigen binding fragment thereof, comprises heavy chain CDR1, CDR2 and CDR3 domains having the sequences set forth in SEQ ID NOs:37, 38 and 39, respectively, and light chain CDR1, CDR2 and CDR3 domains having the sequences set forth in SEQ ID NOs:40, 41 and 42, respectively. In another embodiment, the antibody comprises the VH region of the 305LO5 antibody having the sequence set forth in SEQ ID NO:43, and the VL region of the 305LO5 antibody having the sequence set forth in SEQ ID NO:44.

Another exemplary anti-C5 antibody is the SKY59 antibody (Fukuzawa, T. et al., Sci. Rep., 7:1080, 2017). In one embodiment, the antibody comprises the heavy and light chain CDRs or variable regions of the SKY59 antibody. In another embodiment, the antibody, or antigen binding fragment thereof, comprises a heavy chain comprising SEQ ID NO:45 and a light chain comprising SEQ ID NO:46.

In some embodiments, the anti-C5 antibody comprises the heavy and light chain variable regions or heavy and light chains of the REGN3918 antibody (see U.S. Pat. No. 10,633,434). In some embodiments, the anti-C5 antibody, or antigen-binding fragment thereof, comprises a heavy chain variable region sequence set forth in SEQ ID NO: 47 and a light chain variable region comprising the sequence set forth in SEQ ID NO: 48. In some embodiments, the anti-C5 antibody, or antigen-binding fragment thereof, comprises a heavy chain sequence set forth in SEQ ID NO: 49 and a light chain sequence set forth in SEQ ID NO: 50.

In some embodiments, an anti-C5 antibody described herein comprises a heavy chain CDR1 comprising, or consisting of, the following amino acid sequence: GHIFSNYWIQ (SEQ ID NO:19). In some embodiments, an anti-C5 antibody described herein comprises a heavy chain CDR2 comprising, or consisting of, the following amino acid sequence: EILPGSGHTEYTENFKD (SEQ ID NO:18). In some embodiments, an anti-C5 antibody described herein comprises a heavy chain variable region comprising the following amino acid sequence:

(SEQ ID NO: 12)
QVQLVQSGAE VKKPGASVKV SCKASGHIES NYWIQWVRQA
PGQGLEWMGE ILPGSGHTEY TENFKDRVTM TRDTSTSTVY
MELSSLRSED TAVYYCARYF FGSSPNWYFD VWGQGTLVTV SS.

In some embodiments, an anti-C5 antibody described herein comprises a light chain variable region comprising the following amino acid sequence:

(SEQ ID NO: 8)
DIQMTQSPSS LSASVGDRVT ITCGASENIY GALNWYQQKP
GKAPKLLIYG ATNLADGVPS RFSGSGSGTD FTLTISSLQP
EDFATYYCQN VLNTPLTFGQ GTKVEIK.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. The Fc constant region can, for example, comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., WO2015134894 and U.S. Pat. No. 9,079,949 the disclosures of each of which are incorporated herein by reference in their entirety.

Substitutions that enhance the binding affinity of an antibody Fc constant region for FcRn are known in the art and include, e.g., (1) the M252Y/S254T/T256E triple substitution (Dall'Acqua, W. et al., J. Biol. Chem., 281:23514-24, 2006); (2) the M428L or T250Q/M428L substitutions (Hinton, P. et al., J. Biol. Chem., 279:6213-6, 2004; Hinton, P. et al., J. Immunol., 176:346-56, 2006); and (3) the N434A or T307/E380A/N434A substitutions (Petkova, S. et al., Int. Immunol., 18:1759-69, 2006). The additional substitution pairings: P257I/Q311I, P257I/N434H and D376V/N434H (Datta-Mannan, A. et al., J. Biol. Chem., 282:1709-17, 2007), the disclosures of each of which are incorporated herein by reference in their entirety.

In some embodiments, the variant constant region has a substitution at EU amino acid position 255 for valine. In some embodiments, the variant constant region has a substitution at EU amino acid position 309 for asparagine. In some embodiments, the variant constant region has a substitution at EU amino acid position 312 for isoleucine. In some embodiments, the variant constant region has a substitution at EU amino acid position 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, 9, 8, 7, 6, 5, 4, 3 or 2) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I and V308F. In some embodiments, the variant human Fc constant region comprises a methionine at position 428 and an asparagine at position 434 of a native human IgG Fc constant region, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/434S double substitution when used in a IgG2/4 chimeric Fc may correspond to 429L and 435S as in the M429L and N435S variants found in ravulizumab and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable anti-C5 antibodies for use in the methods described herein, in some embodiments, comprise a heavy chain polypeptide comprising the amino acid sequence set forth in SEQ ID NO:14 and/or a light chain polypeptide comprising the amino acid sequence set forth in SEQ ID NO:11. Alternatively, the anti-C5 antibodies for use in the methods described herein, in some embodiments, comprise a heavy chain polypeptide comprising the amino acid sequence set forth in SEQ ID NO:20 and/or a light chain polypeptide comprising the amino acid sequence set forth in SEQ ID NO: 11.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, or 0.975) nM. In one embodiment, the antibody binds to C5 at pH 7.4 and 25° C. (and, otherwise, under physiologic conditions) with an affinity dissociation constant (K_{D}) that is about 0.5 nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3, or 0.2) nM. In some embodiments, the antibody binds to C5 at pH 6.0 and 25° C. (and, otherwise, under physiologic conditions) with a K_{D} that is about 22 nM.

In other embodiments, the [(K_{D} of the antibody for C5 at pH 6.0 at 25° C.)/(K_{D} of the antibody for C5 at pH 7.4 at 25 C)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000) Methods for determining whether an antibody binds to a protein antigen and/or the affinity for an antibody to a protein antigen are known in the art. The binding of an antibody to a protein antigen, for example, can be detected and/or quantified using a variety of techniques such as, but not limited to, Western blot, dot blot, surface plasmon resonance (SPR) detection (e.g., BIAcore system; Pharmacia Biosensor AB, Uppsala, Sweden and Piscataway, N.J.), or enzyme-linked immunosorbent assay (ELISA; Benny K. C. Lo (2004) “Antibody Engineering: Methods and Protocols,” Humana Press (ISBN: 1588290921); Johne, B. et al., J. Immunol. Meth., 160:191-8, 1993; Jonsson, U. et al., Ann. Biol. Clin., 51:19-26, 1993; Jonsson, U. et al., Biotechniques, 11:620-7, 1991). In addition, methods for measuring the affinity (e.g., dissociation and association constants) are set forth in the working examples.

As used herein, the term “kₐ” refers to the rate constant for association of an antibody to an antigen. The term “kₐ” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/k_{d}. Such determinations can be measured, for example, at 25 C or 37 C (see the working examples). The kinetics of antibody binding to human C5 can be determined, for example, at pH 8.0, 7.4, 7.0, 6.5 and 6.0 via SPR on a BIAcore 3000 instrument using an anti-Fc capture method to immobilize the antibody.

In one embodiment, the anti-C5 antibody, or antigen binding fragment thereof, blocks the cleavage of C5 into C5a and C5b. Through this blocking effect, for example, the pro-inflammatory effects of C5a and the generation of the C5b-9 membrane attack complex (MAC) at the surface of a cell are inhibited.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods known in the art such as, for example, by a conventional hemolytic assay such as the hemolysis assay (Kabat and Mayer (eds.), “Experimental Immunochemistry, 2^{nd} Edition,” 135-240, Springfield, IL, CC Thomas (1961), pages 135-139), or a conventional variation of that assay such as the chicken erythrocyte hemolysis method (Hillmen, P. et al., N. Engl. J Med., 350:552-9, 2004). Methods for determining whether a candidate compound inhibits the cleavage of human C5 into forms C5a and C5b are known in the art (Evans, M. et al., Mol. Immunol., 32:1183-95, 1995). The concentration and/or physiologic activity of C5a and C5b in a body fluid can be measured, for example, by methods known in the art. For C5b, hemolytic assays or assays for soluble C5b-9 as discussed herein can be used. Other assays known in the art can also be used. Using assays of these or other suitable types, candidate agents capable of inhibiting human complement component C5 can be screened.

Immunological techniques such as, but not limited to, ELISA can be used to measure the protein concentration of C5 and/or its split products to determine the ability of an anti-C5 antibody, or antigen binding fragment thereof, to inhibit conversion of C5 into biologically active products. In some embodiments, C5a generation is measured. In some embodiments, C5b-9 neoepitope-specific antibodies are used to detect MAC formation.

Hemolytic assays can be used to determine the inhibitory activity of an anti-C5 antibody, or antigen binding fragment thereof, on complement activation. To determine the effect of an anti-C5 antibody, or antigen binding fragment thereof, on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with an anti-C5 antibody, or antigen binding fragment thereof, in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the anti-C5 antibody, or antigen binding fragment thereof. In some embodiments, the test serum is a C5-deficient serum reconstituted with a C5 polypeptide.

To determine the effect of an anti-C5 antibody, or antigen binding fragment thereof, on alternative pathway-mediated hemolysis, unsensitized rabbit or guinea pig erythrocytes can be used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab© Alternative Pathway Complement Kit (Wieslab® COMPL AP330, Euro-Diagnostica, Sweden). Briefly, the test serum is incubated with an anti-C5 antibody, or antigen binding fragment thereof, in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the anti-C5 antibody, or antigen binding fragment thereof.

In some embodiments, C5 activity, or inhibition thereof, is quantified using a CH50eq assay. The CH50eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50eq assay provides an indirect measure of terminal complement complex (TCC) formation, since the TCC themselves are directly responsible for the hemolysis that is measured. The assay is known and commonly practiced by those of skill in the art. Briefly, to activate the classical complement pathway, undiluted serum samples (e.g., reconstituted human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Inhibition, e.g., as it pertains to terminal complement activity, includes at least a 5 (e.g., at least a 6, 7, 8, 9, 10, 15, 20, 25, 30, 35, 40, 45, 50, 55 or 60) % decrease in the activity of terminal complement in, e.g., a hemolytic assay or CH50eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least 40 (e.g., at least 45, 50, 55, 60, 65, 70, 75, 80, 85, 90, or 95 or greater) %. In some embodiments, an anti-C5 antibody described herein contains one or more amino acid substitutions relative to the CDRs of eculizumab (i.e., SEQ ID NOs:1-6), yet retains at least 30 (e.g., at least 31, 32, 33, 34, 35, 36, 37, 38, 39, 40, 41, 42, 43, 44, 45, 46, 47, 48, 49, 50, 55, 60, 65, 70, 75, 80, 85, 90 or 95) % of the complement inhibitory activity of eculizumab in a hemolytic assay or CH50eq assay.

An anti-C5 antibody described herein has a serum half-life in humans that is at least 20 (e.g., at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55) days. In another embodiment, the anti-C5 antibody described herein has a serum half-life in humans that is at least 40 days. In another embodiment, the anti-C5 antibody described herein has a serum half-life in humans that is approximately 43 days. In another embodiment, the anti-C5 antibody described herein has a serum half-life in humans that is between 39-48 days. Methods for measuring the serum half-life of an antibody are known in the art. In some embodiments, an anti-C5 antibody, or antigen binding fragment thereof, described herein has a serum half-life that is at least 20 (e.g., at least 30, 35, 40, 45, 50, 55, 60, 65, 70, 75, 80, 85, 90, 95, 100, 125, 150, 175, 200, 250, 300, 400 or 500) % greater than the serum half-life of eculizumab, e.g., as measured in one of the mouse model systems described in the working examples (e.g., the C5-deficient/NOD/scid mouse or hFcRn transgenic mouse model system).

In one embodiment, the antibody competes for binding with, and/or binds to the same epitope on C5 as an antibody described herein. The term “binds to the same epitope” with reference to two or more antibodies means that the antibodies bind to the same segment of amino acid residues, as determined by a given method. Techniques for determining whether antibodies bind to the same epitope on C5 with an antibody described herein include, for example, epitope mapping methods, such as, x-ray analyses of crystals of antigen:antibody complexes, and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, CDR2 and CDR3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the antibody that is incubated first with the target). Competing antibodies can bind to, for example, the same epitope, an overlapping epitope or to adjacent epitopes (e.g., as evidenced by steric hindrance).

Anti-C5 antibodies, or antigen-binding fragments thereof described herein, used in the methods described herein can be generated using a variety of art-recognized techniques. Monoclonal antibodies can be obtained by various techniques familiar to those skilled in the art. Briefly, spleen cells from an animal immunized with a desired antigen are immortalized, commonly by fusion with a myeloma cell (Kohler, G. & Milstein, C., Eur. J Immunol., 6:511-9, 1976)). Methods of immortalization include transformation with Epstein Barr Virus, oncogenes, or retroviruses or other methods known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a binding fragment thereof by screening a DNA library from human B cells (Huse, W. et al., Science, 246:1275-81, 1989).

In some embodiments, the anti-C5 antibody does not comprise eculizumab (SOLIRIS®) or an antigen-binding fragment thereof (e.g., comprising heavy and light chain complementarity determining regions (HCDR₁₋₃ and LCDR₁₋₃, respectively) of eculizumab). In some embodiments, the anti-C5 antibody is not a biosimilar of eculizumab (SOLIRIS®), e.g., ABP 959 antibody (manufactured by Amgen Inc., USA), ELIZARIA® (manufactured by Generium JNC, Russia), or SB12 (manufactured by Samsung Bioepis, Incheon, South Korea).

### III. Compositions

Also provided herein are compositions comprising an anti-C5 antibody or antigen binding fragment thereof. In one embodiment, the composition comprises an anti-C5 antibody comprising the CDR1, CDR2 and CDR3 domains in a heavy chain variable region having the sequence set forth in SEQ ID NO:12, and the CDR1, CDR2 and CDR3 domains in a light chain variable region having the sequence set forth in SEQ ID NO:8. In another embodiment, the anti-C5 antibody comprises heavy and light chains having the sequences shown in SEQ ID NOs:14 and 11, respectively. In another embodiment, the anti-C5 antibody comprises heavy and light chains having the sequences shown in SEQ ID NOs:20 and 11, respectively.

The compositions can be formulated as a pharmaceutical solution, e.g., for administration to a subject for the treatment of HSCT-TMA. The pharmaceutical compositions generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt, sugars, carbohydrates, polyols and/or tonicity modifiers.

The compositions can be formulated according to standard methods. Pharmaceutical formulation is an established art (see, for example, Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X)). In some embodiments, a composition can be formulated, for example, as a buffered solution at a suitable concentration and suitable for storage at 2-8 C (e.g., 4 C). In some embodiments, a composition can be formulated for storage at a temperature below OC (e.g., −20 C or −80 C). In some embodiments, the composition can be formulated for storage for up to 2 years (e.g., 1 month, 2 months, 3 months, 4 months, 5 months, 6 months, 7 months, 8 months, 9 months, 10 months, 11 months, 1 year, 1½ years or 2 years) at 2-8 C (e.g., 4 C). Thus, in some embodiments, the compositions described herein are stable in storage for at least 1 year at 2-8 C (e.g., 4 C). The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. Compositions containing a composition intended for systemic or local delivery, for example, can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

In some embodiments, the disclosure relates to a composition, e.g., pharmaceutical composition or a medicament, comprising an effective amount of an anti-C5 antibody or an antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, 5 and 6, respectively, for use in the treatment of hematopoietic stem cell transplant-associated thrombotic microangiopathy (HSCT-TMA), e.g., TMA after HSCT, in a human patient, wherein the effective amount comprises administration of the anti-C5 antibody or the antigen binding fragment thereof: (a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; (b) once on Day 5 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; (c) once on Day 10 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and (d) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg. In some embodiments, the disclosure relates to a composition, e.g., pharmaceutical composition or a medicament, comprising an effective amount of an anti-C5 antibody or antigen binding fragment thereof, comprising HCDR₁₋₃ comprising SEQ ID NOs:19, 18 and 3 and LCDR₁₋₃ comprising SEQ ID NOs: 4, 5 and 6, for use in the treatment of HSCT-TMA, e.g., TMA after HSCT, in a human patient, wherein the anti-C5 antibody further comprises a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering. Particularly, the disclosure relates to a pharmaceutical composition or a medicament comprising an effective amount of ravulizumab (ULTOMIRIS®) or an antigen-binding fragment thereof, e.g., comprising the HCDR₁₋₃ and the LCDR₁₋₃ of ravulizumab, for use in the treatment of HSCT-TMA, e.g., TMA after HSCT, in a human patient.

### IV. Methods

Provided herein are methods for treating HSCT-TMA in a human patient, comprising administering to the patient an anti-C5 antibody, or antigen binding fragment thereof, wherein the anti-C5 antibody or antigen binding fragment thereof is administered (or is for administration) according to a particular clinical dosage regimen (e.g., at a particular dose amount and according to a specific dosing schedule).

In one embodiment, the dose of the anti-C5 antibody, or antigen binding fragment thereof, is based on the weight of the patient. In one embodiment, for example, 300 mg or 600 mg of the anti-C5 antibody, or antigen binding fragment thereof, is administered to a patient weighing ≥5 to <10 kg. In another embodiment, 300 mg or 600 mg of the anti-C5 antibody, or antigen binding fragment thereof, is administered to a patient weighing ≥10 to <20 kg. In another embodiment, 300 mg, 900 mg, or 2100 mg of the anti-C5 antibody, or antigen binding fragment thereof, is administered to a patient weighing ≥20 to <30 kg. In another embodiment, 300 mg, 1200 mg, or 2700 mg of the anti-C5 antibody, or antigen binding fragment thereof, is administered to a patient weighing ≥30 to <40 kg. In another embodiment, 600 mg, 2400 mg, or 3000 mg of the anti-C5 antibody, or antigen binding fragment thereof, is administered to a patient weighing ≥40 to <60 kg. In another embodiment, 900 mg, 2700 mg, or 3300 mg of the anti-C5 antibody, or antigen binding fragment thereof, is administered to a patient weighing ≥60 to <100 kg. In another embodiment, 900 mg, 3000 mg, or 3600 mg of the anti-C5 antibody, or antigen binding fragment thereof, is administered to a patient weighing ≥100 kg. In certain embodiments, dosage regimens are adjusted to provide the optimum desired response (e.g., an effective response).

In another embodiment, the anti-C5 antibody, or antigen binding fragment thereof, is administered for one or more administration cycles. In one embodiment, the treatment (e.g., administration cycle) is 26 weeks. In one embodiment, the anti-C5 antibody, or antigen binding fragment thereof, is administered once on Day 1 (e.g., of the administration cycle), once on Day 5 (e.g., of the administration cycle), once on Day 10 (e.g., of the administration cycle). In another embodiment, the anti-C5 antibody, or antigen binding fragment thereof, is administered is administered once on Day 1 (e.g., of the administration cycle), once on Day 5 (e.g., of the administration cycle), once on Day 10 (e.g., of the administration cycle) and every eight four or eight weeks thereafter starting at Day 15. In another embodiment, the anti-C5 antibody, or antigen binding fragment thereof, is administered every four or eight weeks after treatment (e.g., an administration cycle) for an extension period up to two years (e.g., at a dose of 300 mg, 600 mg, 900 mg, 1200 mg, 2100 mg, 2400 mg, 2700 mg, 3000 mg, 3300 mg or 3600 mg).

In another embodiment, a method of treating a human patient with HSCT-TMA is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In another embodiment, a method of treating a human patient with HSCT-TMA is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg; and
   wherein the treatment results in a reduction in LDH levels to within normal levels or to within 50% below what is considered the ULN level (e.g., within 105-333 IU/L (international units per liter), a serum trough concentration of the anti-C5 antibody, or antigen binding fragment thereof, of at least 175 μg/mL or greater, and/or a free C5 concentration of 0.5 μg/mL or less (e.g., 0.4 μg/mL, 0.3 μg/mL, 0.2 μg/mL, or 0.1 μg/mL or less).

In another embodiment, a method of treating a human patient with HSCT-TMA is provided, the method comprising administering to the patient (e.g., during an administration cycle) an effective amount of an anti-C5 antibody, or antigen binding fragment thereof, comprising CDR1, CDR2 and CDR3 heavy chain sequences as set forth in SEQ ID NOs:19, 18 and 3, respectively, CDR1, CDR2 and CDR3 light chain sequences as set forth in SEQ ID NOs:4, 5 and 6, respectively, and a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention, wherein the anti-C5 antibody, or antigen binding fragment thereof, is administered:
(a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In another embodiment, the anti-C5 antibody or antigen binding fragment thereof is administered to a patient weighing ≥5 to <10 kg:
(a) once on Day 1 at a dose of 600 mg;
(b) once on Day 5 at a dose of 300 mg;
(c) once on Day 10 at a dose of 300 mg; and
(d) on Day 15 and every four weeks thereafter at a dose of 300 mg.

In another embodiment, the anti-C5 antibody is administered to a patient weighing ≥10 to <20 kg:
(a) once on Day 1 at a dose of 600 mg;
(b) once on Day 5 at a dose of 300 mg;
(c) once on Day 10 at a dose of 300 mg; and
(d) on Day 15 and every four weeks thereafter at a dose of 600 mg.

In another embodiment, the anti-C5 antibody is administered to a patient weighing ≥20 to <30 kg:
(a) once on Day 1 at a dose of 900 mg;
(b) once on Day 5 at a dose of 300 mg;
(c) once on Day 10 at a dose of 300 mg; and
(d) on Day 15 and every eight weeks thereafter at a dose of 2100 mg.

In another embodiment, the anti-C5 antibody is administered to a patient weighing ≥30 to <40 kg:
(a) once on Day 1 at a dose of 1200 mg;
(b) once on Day 5 at a dose of 300 mg;
(c) once on Day 10 at a dose of 300 mg; and
(d) on Day 15 and every eight weeks thereafter at a dose of 2700 mg.

In another embodiment, the anti-C5 antibody is administered to a patient weighing ≥40 to <60 kg:
(a) once on Day 1 at a dose of 2400 mg;
(b) once on Day 5 at a dose of 600 mg;
(c) once on Day 10 at a dose of 600 mg; and
(d) on Day 15 and every eight weeks thereafter at a dose of 3000 mg.

In another embodiment, the anti-C5 antibody is administered to a patient weighing ≥60 to <100 kg:
(a) once on Day 1 at a dose of 2700 mg;
(b) once on Day 5 at a dose of 900 mg;
(c) once on Day 10 at a dose of 900 mg; and
(d) on Day 15 and every eight weeks thereafter at a dose of 3300 mg.

In another embodiment, the anti-C5 antibody is administered to a patient weighing ≥100 kg:
(a) once on Day 1 at a dose of 3000 mg;
(b) once on Day 5 at a dose of 900 mg;
(c) once on Day 10 at a dose of 900 mg; and
(d) on Day 15 and every eight weeks thereafter at a dose of 3600 mg.

In one embodiment, the methods of treatment disclosed herein further comprise administering best supportive care (BSC) measures. BSC measures include, but are not limited to, transfusion support, corticosteroids, dialysis, and antihypertensive medications. In one embodiment, the patient has not previously been treated with eculizumab. In another embodiment, the patient has previously been treated with eculizumab. In another embodiment, the patient has previously been treated with eculizumab and Day 1 (e.g., of the administration cycle) is two weeks or more from the patient's last dose of eculizumab.

In some embodiments, the disclosure relates to use of an effective amount of an anti-C5 antibody or antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, 5 and 6, respectively, in the manufacture of a composition, e.g., pharmaceutical composition or a medicament, for treating hematopoietic stem cell transplant-associated thrombotic microangiopathy (HSCT-TMA), e.g., TMA after HSCT, in a human patient, wherein the effective amount comprises administration of the anti-C5 antibody or the antigen binding fragment thereof: (a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg; (b) once on Day 5 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; (c) once on Day 10 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and (d) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg. In some embodiments, the disclosure relates to use of an effective amount of an anti-C5 antibody or antigen binding fragment thereof comprising HCDR₁₋₃ comprising SEQ ID NOs:19, 18 and 3 and LCDR₁₋₃ comprising SEQ ID NOs: 4, 5 and 6, in the manufacture of a composition, e.g., pharmaceutical composition or a medicament, for treating HSCT-TMA, e.g., TMA after HSCT, in a human patient, wherein the anti-C5 antibody further comprises a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering. Particularly, the disclosure relates to use of an effective amount of ravulizumab (ULTOMIRIS®) or an antigen-binding fragment thereof, e.g., comprising the HCDR₁₋₃ and the LCDR₁₋₃ of ravulizumab, in the manufacture of a composition, e.g., pharmaceutical composition or a medicament, for treating HSCT-TMA, e.g., TMA after HSCT, in a human patient.

### V. Outcomes

Provided herein are methods for treating HSCT-TMA in a patient comprising administering to the patient an anti-C5 antibody. Symptoms of HSCT-TMA include, but are not limited to, e.g., microangiopathic hemolytic anemia, thrombocytopenia, endothelial injury, kidney damage, kidney failure, serositis, pulmonary hypertension, and multisystem organ failure.

Patients treated according to the methods disclosed herein experience improvement in at least one sign of HSCT-TMA. The treatment may produce at least one therapeutic effect selected from the group consisting of, for example, a reduction or cessation in microangiopathic hemolytic anemia, thrombocytopenia, endothelial injury, kidney damage, kidney failure, serositis, pulmonary hypertension, and multisystem organ failure compared to baseline.

In another embodiment, the treatment results in (a) platelet count ≥50,000/mm³ without transfusion support during the prior 7 days, (b) LDH<1.5×ULN, and (c) absence of schistocytes (if there were schistocytes present at baseline).

In another embodiment, the treatment results in (a) platelet count ≥50,000/mm³ without transfusion support during the prior 7 days, (b) LDH<1.5×ULN, (c) absence of schistocytes (if there were schistocytes present at baseline) and (d) at least 50% reduction of proteinuria from baseline. In one embodiment, proteinuria is as protein/creatinine ratio ≥0.5 mg/mg.

In another embodiment, the treatment results in a favorable hematological response.

In another embodiment, the treatment results in normalization of LDH, resolution of need for red cell and platelet transfusions, and disappearance of schistocytes.

In another embodiment, the treatment results in the patient's ability to maintain hemoglobin ≥8 g/dL without transfusion support. In another embodiment, the treatment results in hemoglobin ≥8 g/dL without transfusion support.

In another embodiment, the treatment results in a decrease in LDH, an increase in platelets, and/or an increase in hemoglobin compared to baseline.

In another embodiment, the treatment results in normal levels of serum creatinine compared to baseline.

In another embodiment, the treatment results in an improvement in TMA-associated organ dysfunction in the renal, cardiovascular, pulmonary, CNS, and/or GI systems compared to baseline.

In another embodiment, the treatment results in terminal complement inhibition.

In another embodiment, the treatment produces a reduction in adverse events.

In another embodiment, the treatment produces a shift toward normal levels of biomarkers associated with vascular inflammation (e.g., shed tumor necrosis factor receptor 1 [TNF-R1]), endothelial damage and/or activation (e.g., thrombomodulin and shed vascular cell adhesion molecule 1 [VCAM-1]), renal injury (e.g., Cystatin C), and/or complement proteins and complement activation pathway products.

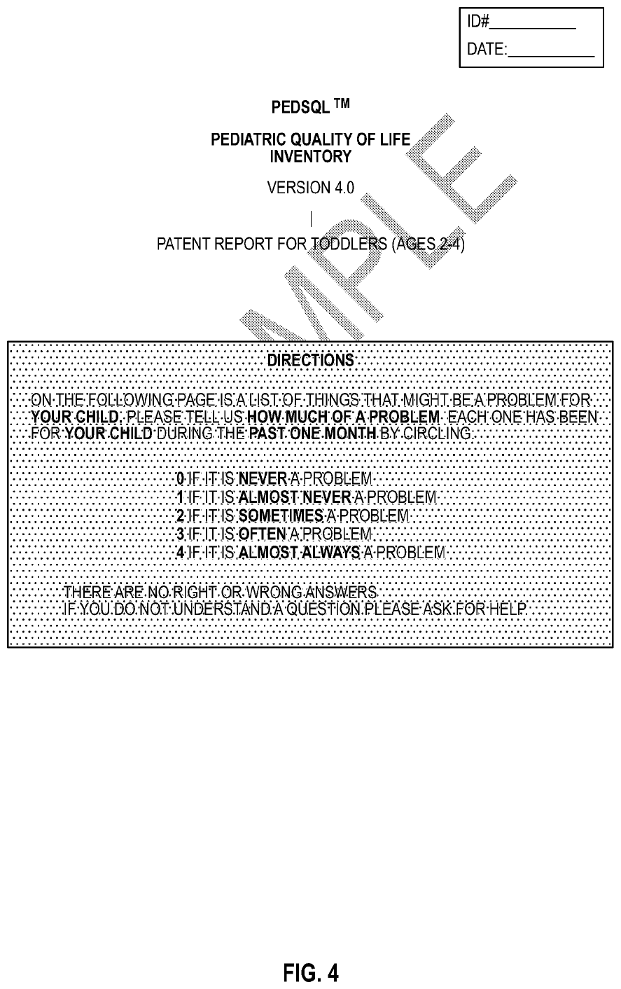

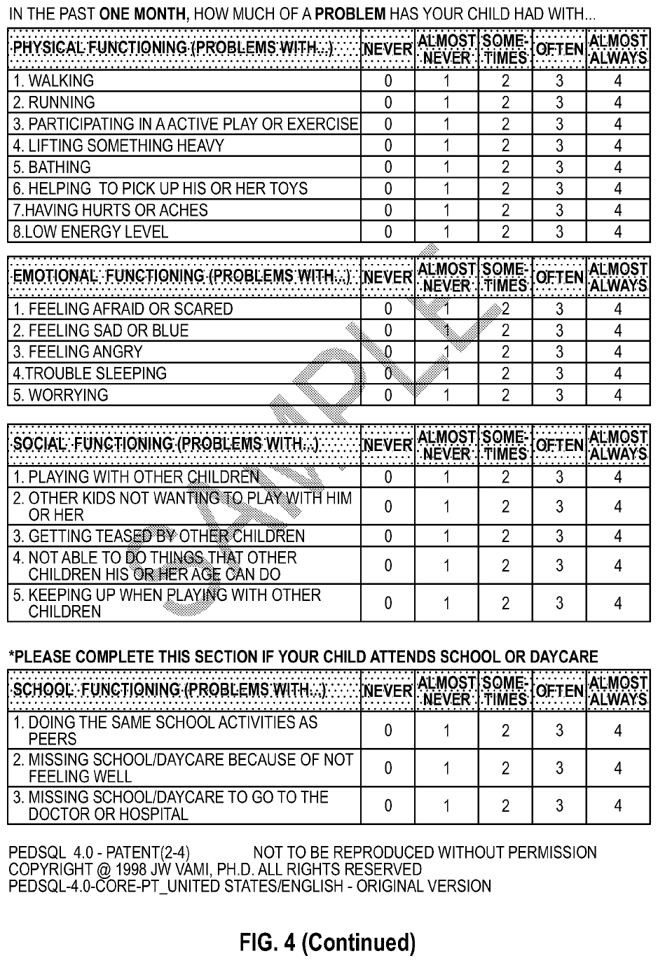

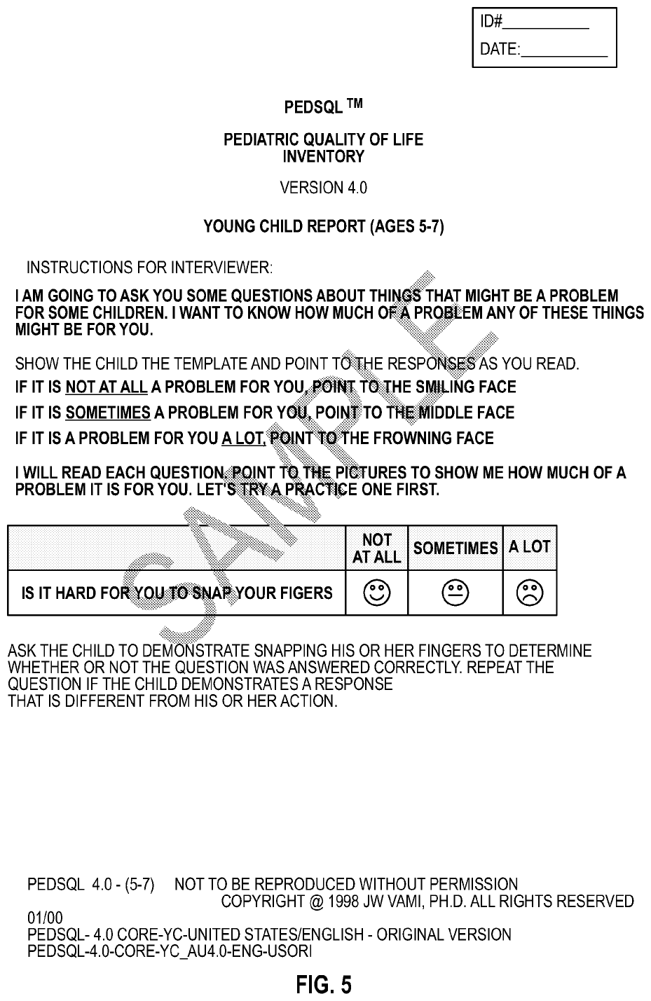

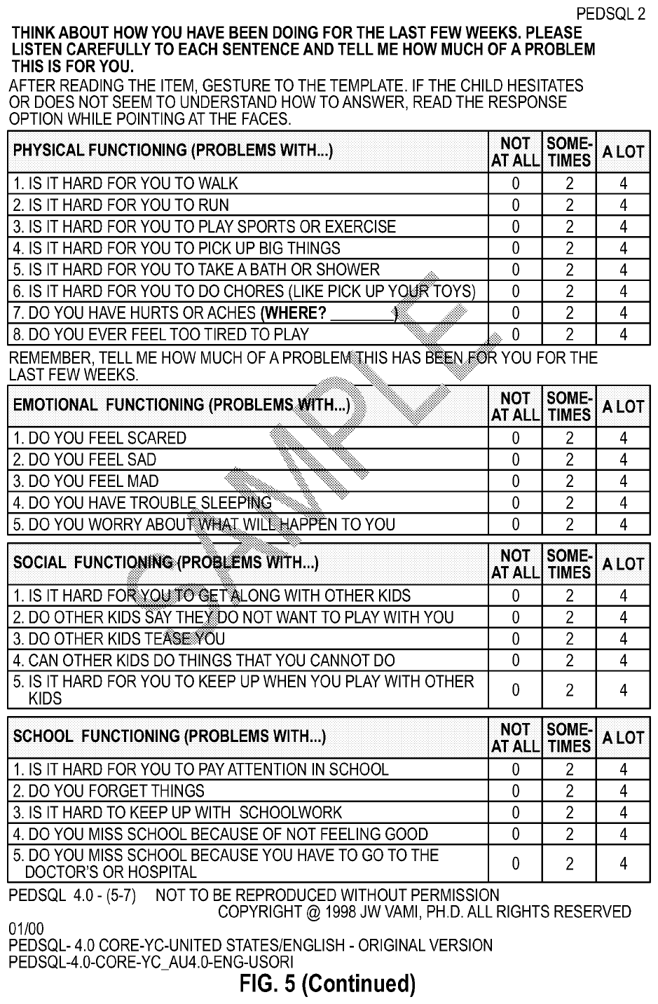

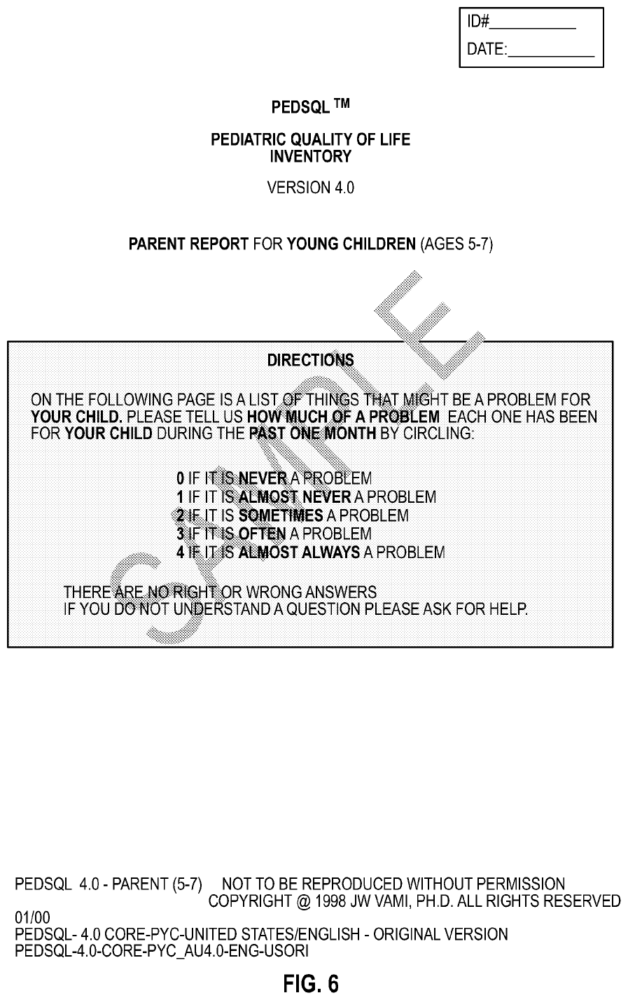

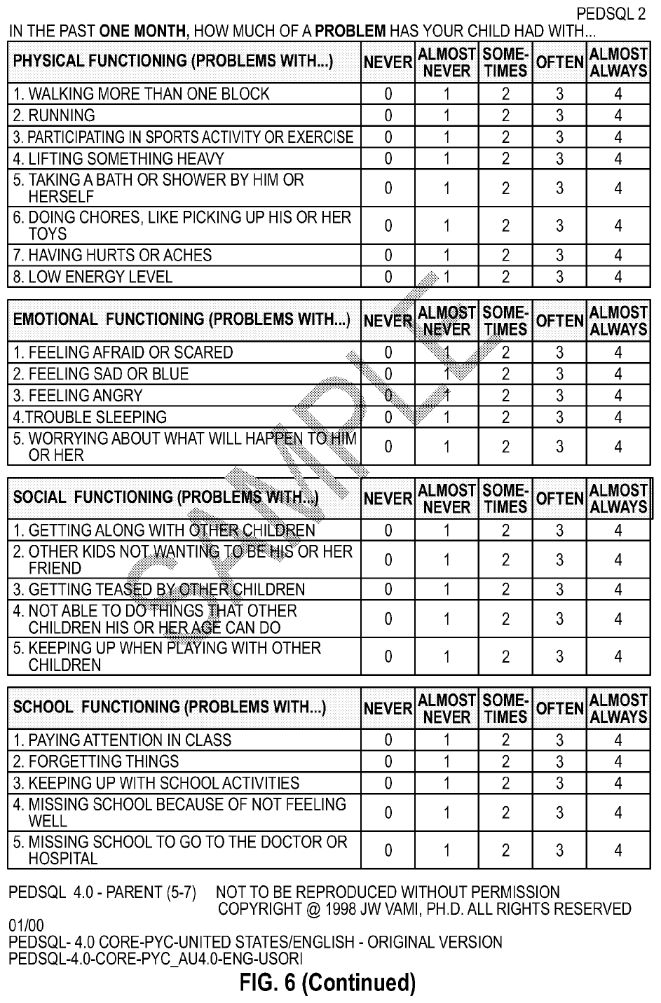

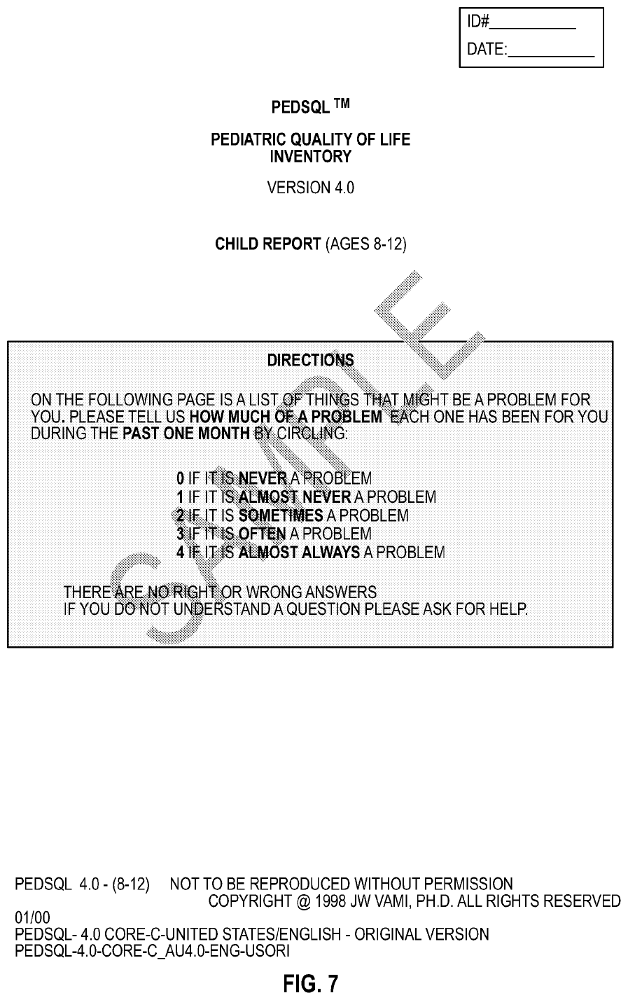

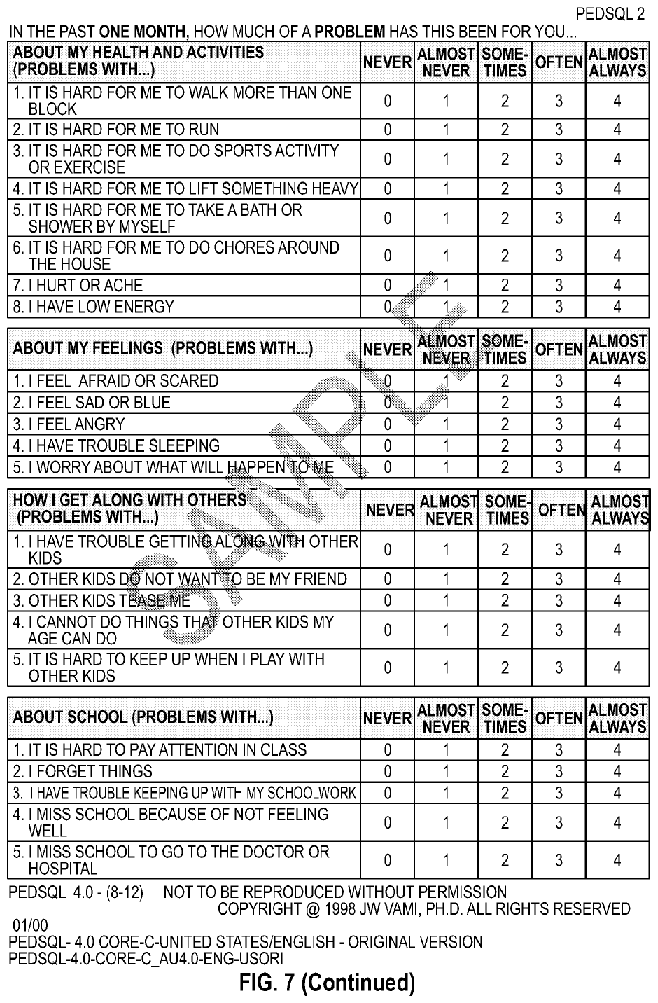

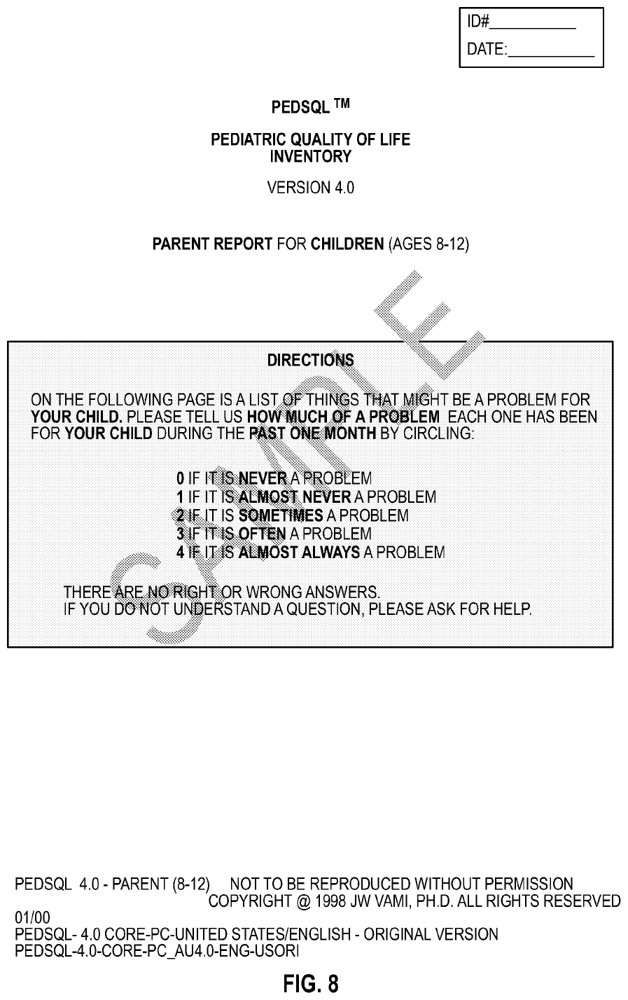

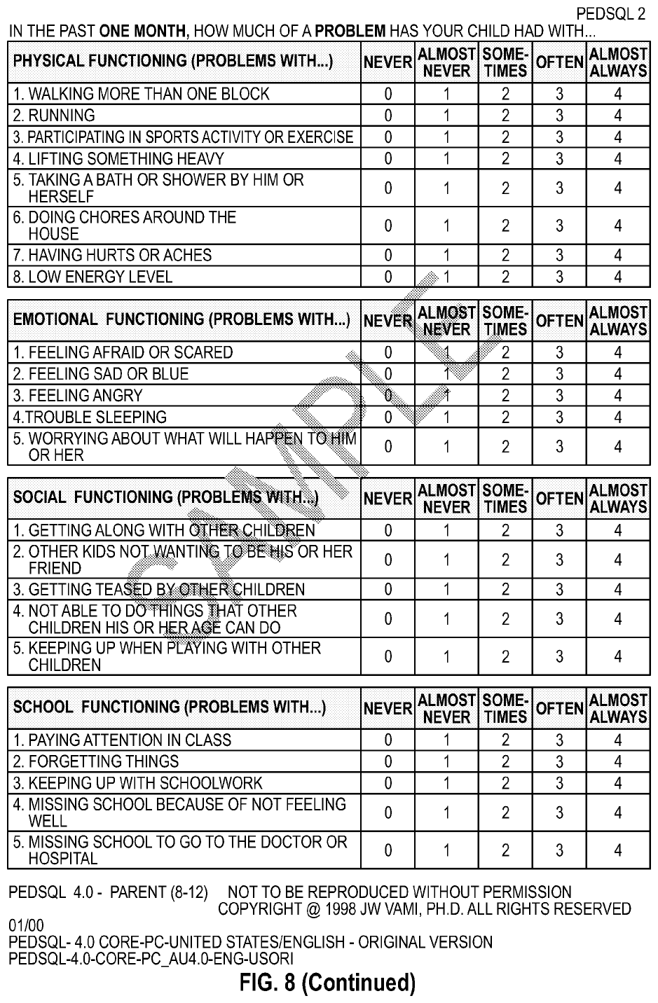

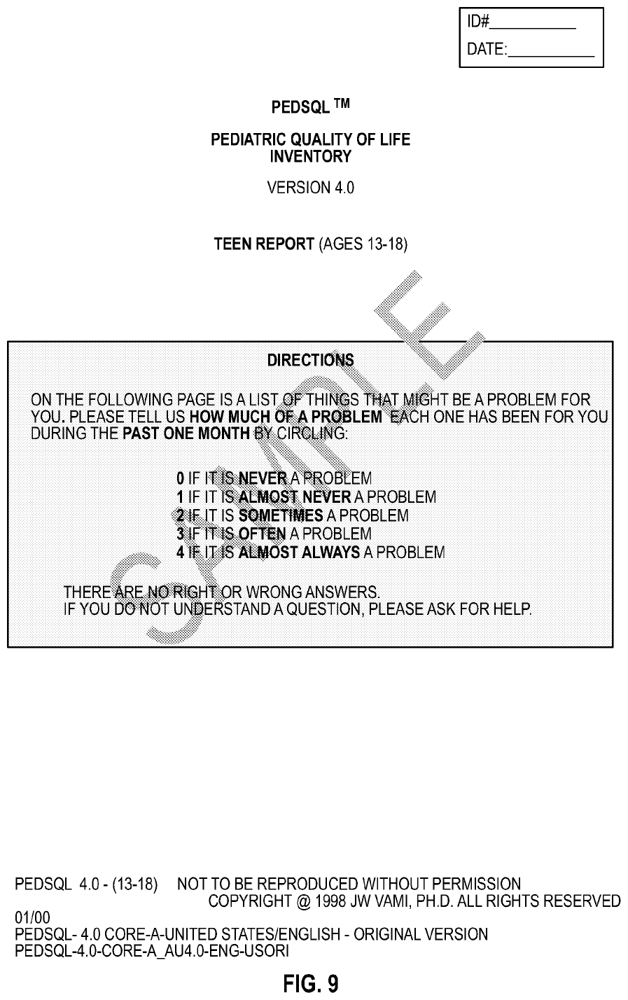

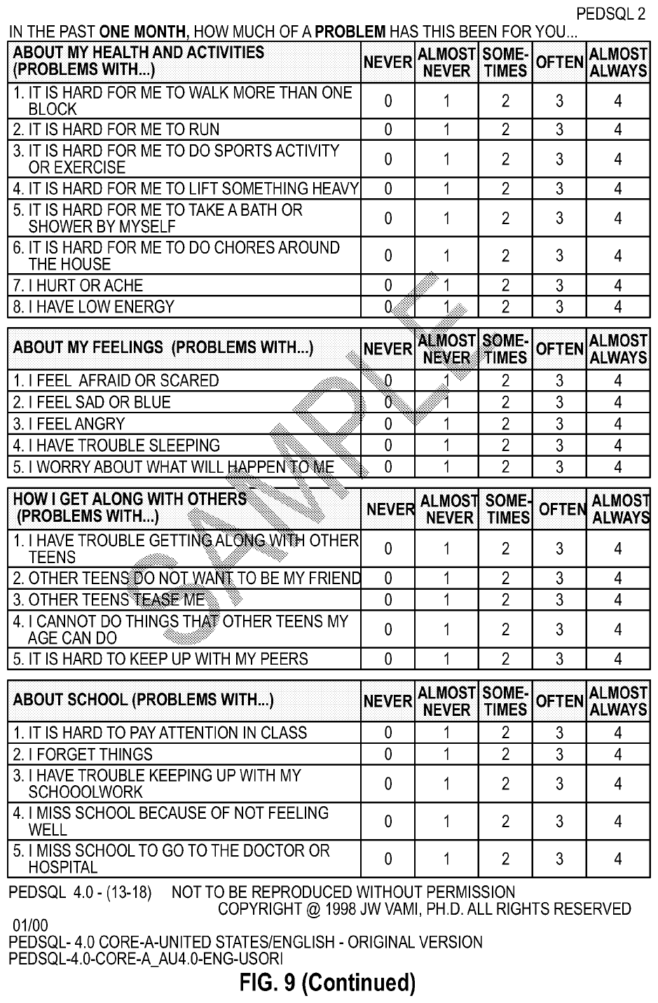

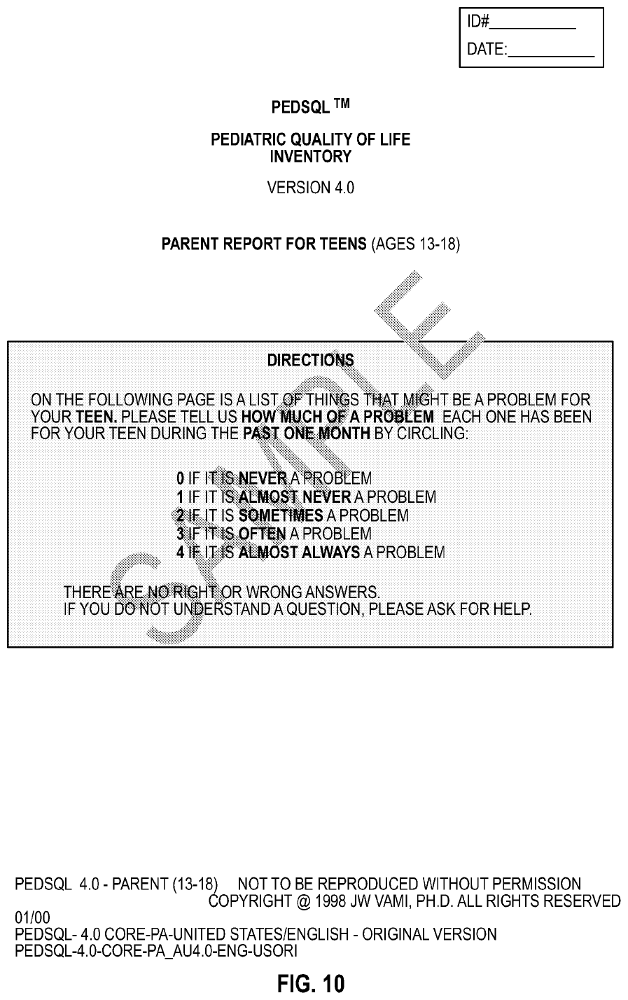

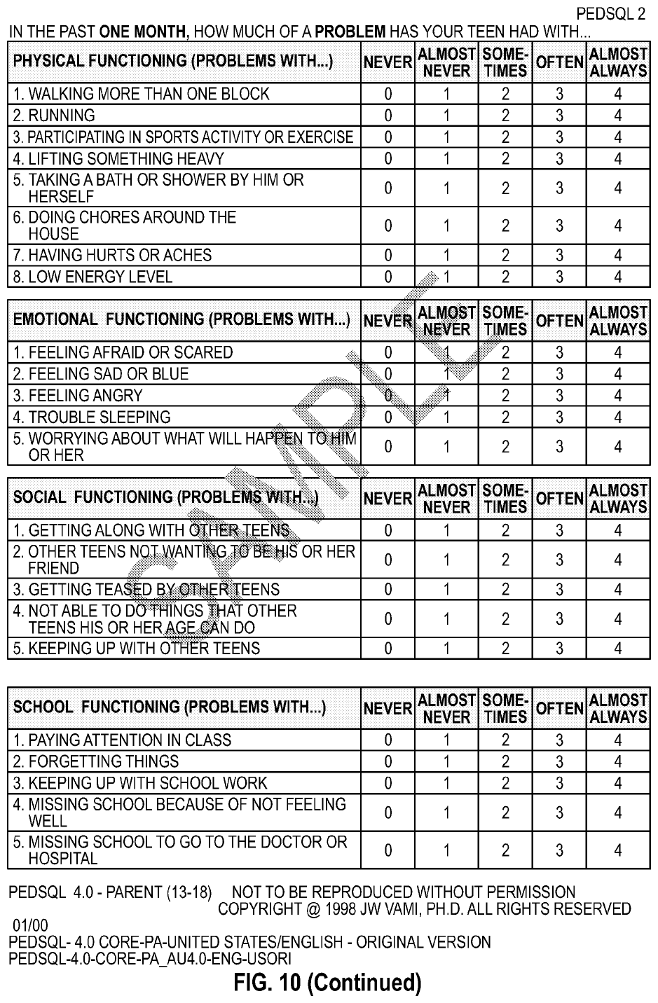

In another embodiment, the treatment produces a change from baseline in quality of life as assessed via a Quality of Life Assessment (e.g., a Quality of Life Inventory (PedsQL) Scale or an EQ-5D-5L questionnaire). Exemplary are Quality of Life Assessment are set forth in FIGS. 4-10 and 15.

In another embodiment, lactate dehydrogenase (LDH) levels can be used to evaluate responsiveness to a therapy. LDH is a marker of intravascular hemolysis (Hill, A. et al., Br. J Haematol., 149:414-25, 2010; Hillmen, P. et al., N. Engl. J Med., 350:552-9, 2004; Parker, C. et al., Blood, 106:3699-709, 2005). Red blood cells contain large amounts of LDH, and a correlation between cell-free hemoglobin and LDH concentration has been reported in vitro (Van Lente, F. et al., Clin. Chem., 27:1453-5, 1981) and in vivo (Kato, G. et al., Blood, 107:2279-85, 2006). The consequences of hemolysis are independent of anemia (Hill, A. et al., Haematologica, 93(s1):359 Abs.0903, 2008; Kanakura, Y. et al., Int. J. Hematol., 93:36-46, 2011). LDH concentration obtained at baseline and then serially throughout a treatment period, is an important measure of hemolysis. The normal LDH value range is 105-333 IU/L (international units per liter).

LDH levels can be measured using any suitable test or assay, such as those described by Ferri F F, ed. Ferri's Clinical Advisor 2014. Philadelphia: Pa: Elsevier Mosby; 2014: Section IV— Laboratory tests and interpretation of results. LDH concentration can be measured in various samples obtained from a patient, in particular, serum samples. As used herein, the term “sample” refers to biological material from a subject. Although serum LDH concentration is of interest, samples can be derived from other sources, including, for example, single cells, multiple cells, tissues, tumors, biological fluids, biological molecules or supernatants or extracts of any of the foregoing. Examples include tissue removed for biopsy, tissue removed during resection, blood, urine, lymph tissue, lymph fluid, cerebrospinal fluid, mucous and stool samples. The sample used can vary based on the assay format, the detection method and the nature of the tumors, tissues, cells or extracts to be assayed. Methods for preparing samples are known in the art and can be readily adapted to obtain a sample that is compatible with the method utilized.

In one embodiment, patients treated according to the disclosed methods experience reductions in LDH levels to normal levels or to within 10%, or within 20% above what is considered the normal level (e.g., within 105-333 IU/L). For example, patients treated according to the disclosed methods experience reductions in LDH levels to within normal levels or to within 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or within 50% below what is considered the ULN level (e.g., within 105-333 IU/L (international units per liter). In one embodiment, the patient's LDH levels are ≥1.5 fold above the ULN (LDH≥1.5×ULN) prior to initiating treatment.

In one embodiment, patients treated according to the disclosed methods experience an LDH percent change compared to baseline of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53% 54%, 55%, 56%, 57%, 58%, 59%, or 60%.

In one embodiment, patients treated according to the disclosed methods maintain a serum trough concentration of the anti-C5 antibody, or antigen binding fragment thereof, of at least 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395 or 400 μg/mL or greater. In one embodiment, patients treated according to the disclosed methods maintain a serum trough concentration of the anti-C5 antibody, or antigen binding fragment thereof, of at least 175 μg/mL or greater.

In one embodiment, patients treated according to the disclosed methods have a free C5 concentration of 0.5 μg/mL or less (e.g., 0.4 μg/mL, 0.3 μg/mL, 0.2 μg/mL, or 0.1 μg/mL or less).

### VI. Kits and Unit Dosage Forms

Also provided herein are kits that include a pharmaceutical composition containing an anti-C5 antibody or antigen binding fragment thereof, such as ravulizumab or BNJ421, and a pharmaceutically acceptable carrier, in a therapeutically effective amount adapted for use in the preceding methods. The kits optionally also can include instructions, e.g., comprising administration schedules, to allow a practitioner (e.g., a physician, nurse, or patient) to administer the composition contained therein to administer the composition to a patient having HSCT-TMA. The kit also can include a syringe.

Optionally, the kits include multiple packages of the single-dose pharmaceutical compositions each containing an effective amount of the anti-C5 antibody, or antigen binding fragment thereof, for a single administration in accordance with the methods provided above. Instruments or devices necessary for administering the pharmaceutical composition(s) also may be included in the kits. For instance, a kit may provide one or more pre-filled syringes containing an amount of the anti-C5 antibody or antigen binding fragment thereof.

In one embodiment, a kit for treating HSCT-TMA in a human pediatric patient comprises: (a) a dose of an anti-C5 antibody or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and (b) instructions for using the anti-C5 antibody or antigen binding fragment thereof, according to any of the methods described herein.

In one embodiment, the kit comprises a dose of an anti-C5 antibody or antigen binding fragment thereof, wherein the anti-C5 antibody, or antigen binding fragment thereof, is administered to a patient weighing ≥5 to <10 kg:
(a) once on Day 1 at a dose of 600 mg;
(b) once on Day 5 at a dose of 300 mg;
(c) once on Day 10 at a dose of 300 mg; and
(d) on Day 15 and every four weeks thereafter at a dose of 300 mg.

In another embodiment, the kit comprises a dose of an anti-C5 antibody, or antigen binding fragment thereof, wherein the anti-C5 antibody, or antigen binding fragment thereof, is administered to a patient weighing ≥10 to <20 kg:
(a) once on Day 1 at a dose of 600 mg;
(b) once on Day 5 at a dose of 300 mg;
(c) once on Day 10 at a dose of 300 mg; and
(d) on Day 15 and every four weeks thereafter at a dose of 600 mg.

In another embodiment, the kit comprises a dose of an anti-C5 antibody, or antigen binding fragment thereof, wherein the anti-C5 antibody, or antigen binding fragment thereof, is administered to a patient weighing ≥20 to <30 kg:
(a) once on Day 1 at a dose of 900 mg;
(b) once on Day 5 at a dose of 300 mg;
(c) once on Day 10 at a dose of 300 mg; and
(d) on Day 15 and every eight weeks thereafter at a dose of 2100 mg.

In another embodiment, the kit comprises a dose of an anti-C5 antibody or antigen binding fragment thereof, wherein the anti-C5 antibody, or antigen binding fragment thereof, is administered to a patient weighing ≥30 to <40 kg:
(a) once on Day 1 at a dose of 1200 mg;
(b) once on Day 5 at a dose of 300 mg;
(c) once on Day 10 at a dose of 300 mg; and
(d) on Day 15 and every eight weeks thereafter at a dose of 2700 mg.

In another embodiment, the kit comprises a dose of an anti-C5 antibody, or antigen binding fragment thereof, wherein the anti-C5 antibody, or antigen binding fragment thereof, is administered to a patient weighing ≥40 to <60 kg:
(a) once on Day 1 at a dose of 2400 mg;
(b) once on Day 5 at a dose of 600 mg;
(c) once on Day 10 at a dose of 600 mg; and
(d) on Day 15 and every eight weeks thereafter at a dose of 3000 mg.

In another embodiment, the kit comprises a dose of an anti-C5 antibody or antigen binding fragment thereof, wherein the anti-C5 antibody, or antigen binding fragment thereof, is administered to a patient weighing ≥60 to <100 kg:
(a) once on Day 1 at a dose of 2700 mg;
(b) once on Day 5 at a dose of 900 mg;
(c) once on Day 10 at a dose of 900 mg; and
(d) on Day 15 and every eight weeks thereafter at a dose of 3300 mg.

In another embodiment, the kit comprises a dose of an anti-C5 antibody, or antigen binding fragment thereof, wherein the anti-C5 antibody, or antigen binding fragment thereof, is administered to a patient weighing ≥100 kg:
(a) once on Day 1 at a dose of 3000 mg;
(b) once on Day 5 at a dose of 900 mg;
(c) once on Day 10 at a dose of 900 mg; and
(d) on Day 15 and every eight weeks thereafter at a dose of 3600 mg.

The following examples are merely illustrative and should not be construed as limiting the scope of this disclosure in any way as many variations and equivalents will become apparent to those skilled in the art upon reading the present disclosure. The contents of all references, Genbank entries, patents and published patent applications cited throughout this application are expressly incorporated herein by reference.

### EXAMPLES

### Example 1: A Phase 3, Open-Label, Single Arm, Multicenter Study of Ravulizumab in Addition to Best Supportive Care in Pediatric Participants (from 1 Month to <18 Years of Age) with Thrombotic Microangiopathy (TMA) after Hematopoietic Stem Cell Transplantation (HSCT)

An open-label, single arm, multicenter study is conducted to evaluate the safety, efficacy, PK, and PD of ravulizumab (ULTOMIRIS®) administered by intravenous (IV) infusion to pediatric participants, from 1 month to <18 years of age, with HSCT TMA. Participants with HSCT-TMA in whom the TMA persists for at least 72 hours after withdrawal or modification of any TMA triggering agent and/or treatment of any underlying trigger conditions are enrolled.

### 1. Objectives

The primary objective of the study is to assess the efficacy of ravulizumab plus best supportive care (BSC) in the treatment of pediatric participants with HSCT TMA via patient TMA response during a 26-week Treatment Period. TMA response is defined as meeting all of the following: (a) Platelet count ≥50,000/mm³ without transfusion support during the prior 7 days, (b) LDH<1.5×ULN and absence of schistocytes (if there were schistocytes present at baseline) and (c) at least 50% reduction of proteinuria from baseline. Participants must meet each response criterion at 2 separate assessments obtained at least 28 days apart, and any measurement in between and all intervals during which each criterion is met must overlap for at least 1 day. Proteinuria is defined as protein/creatinine ratio ≥2 mg/mg.

One secondary objective is to characterize TMA response after treatment with ravulizumab via the following:
a) Time to TMA response (measured in days from Day 1 to the first day that the participant satisfies all the criteria for TMA response);
b) TMA response at Week 26 and at Week 52;
c) Response and time to response for each individual component of TMA response during the 26-week randomized Treatment Period;
d) Hematologic response defined as: Platelet count ≥50,000/mm³ without transfusion support during the prior 7 days and LDH<1.5×ULN and absence of schistocytes (if there were schistocytes present at baseline);
e) Time to hematologic response. Criterion must be met at 2 separate assessments obtained at least 28 days apart, and any measurement in between;
f) Hemoglobin response (ability to maintain hemoglobin ≥8 g/dL without transfusion support: Criterion must be met at 2 separate assessments obtained at least 28 days apart, and any measurement in between);
g) Partial response (participant meets ≥1, but not all, criteria for TMA response); and
h) Loss of TMA response (participant achieves TMA response and, at a subsequent visit, fails to meet the criteria for 1 or more components of TMA response, with confirmation by a second laboratory result at least 24 hours apart).

Additional secondary objectives include assessing: a) improvement in organ dysfunction (change from baseline in TMA-associated organ dysfunction in renal system, cardiovascular system, pulmonary system, CNS, and GI system at 6 months and 1 year), b) TMA relapse and duration of response (TMA relapse during the study (participant meets the criteria for TMA diagnosis after achieving TMA response and duration of TMA response), c) overall survival (at 6 months and 1 year), d) non-relapse mortality (non-relapse mortality (defined as participant's death due to any cause during the study, with the exception of death due to underlying disease progression or relapse), and e) platelet response (platelet response defined as platelet count ≥100,000/mm3 without transfusion support during the prior 7 days; criterion must be met at 2 separate assessments obtained at least 28 days apart, and any measurement in between).

Further objectives include assessing the pharmacokinetics (PK)/pharmacodynamics (PD) (e.g., changes in serum concentrations of ravulizumab over time, changes in serum free C5 concentrations over time, and changes in serum total C5 concentrations over time) and characterizing the safety profile of ravulizumab plus BSC in pediatric participants with HSCT-TMA (e.g., incidence of treatment emergent AEs and treatment emergent SAEs, changes from baseline in vital signs and laboratory parameters, and incidence of ADAs and assessment of immunogenicity).

Exploratory Objectives include assessing biomarkers in pediatric participants with HSCT-TMA (exploratory biomarker analyses evaluate changes from baseline in biomarkers, which may include, but are not limited to, markers of vascular inflammation, endothelial activation, renal injury and complement dysregulation), improvement in QoL patient reported outcomes in pediatric participants with HSCT-TMA (change from baseline in QoL as measured by PedsQL), complement pathway genetic mutations in pediatric participants with HSCT TMA (incidence of specific complement dysregulation related mutations), and describing health resource utilization in pediatric participants with HSCT-TM ((1) number, reason, and duration of hospitalizations (including stays in intensive care unit), (2) number and volume of RBC and platelet transfusions, and (3) number of outpatient medical encounters (including physician or emergency room visits) and the underlying reason).

### 2. Overall Study Design

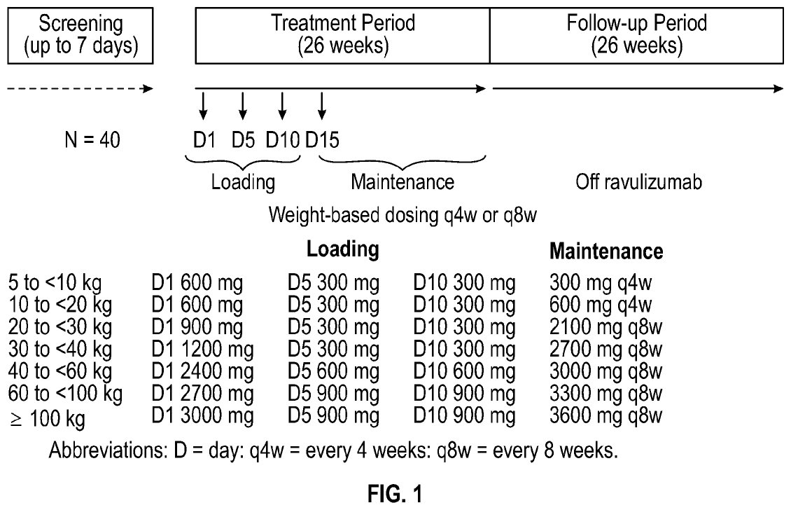

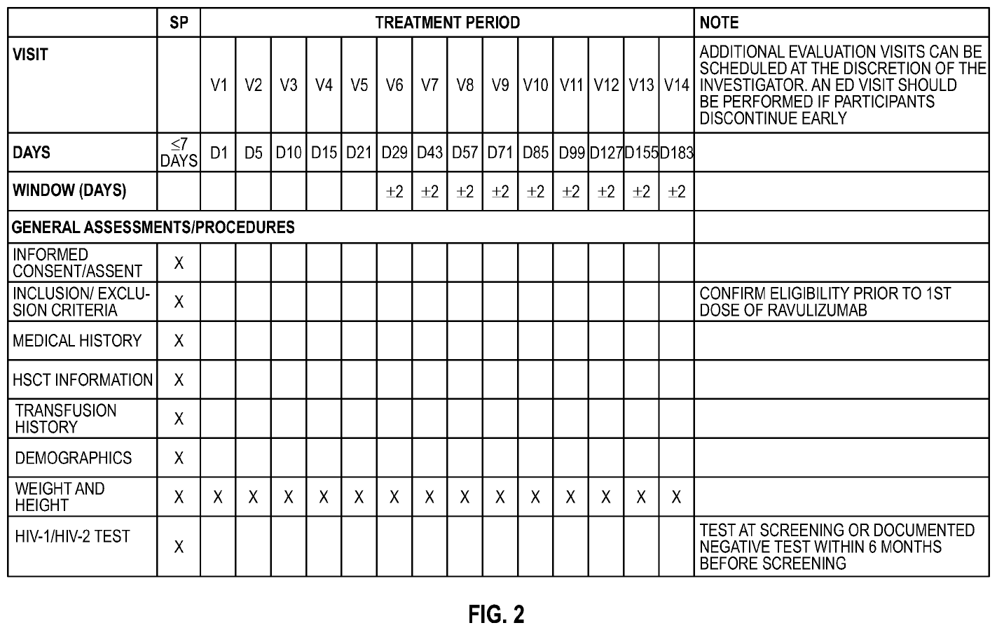

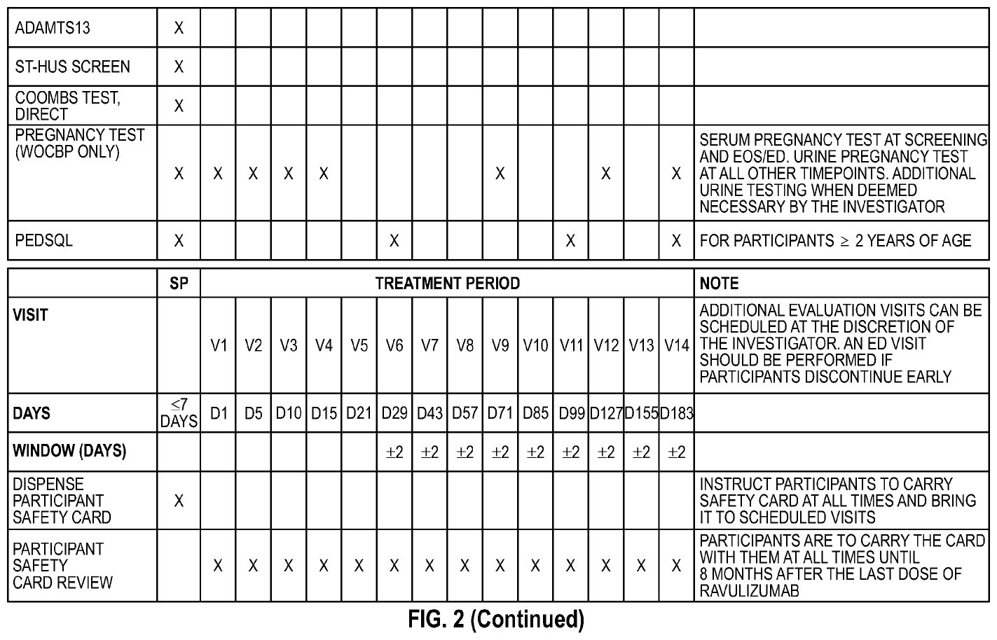

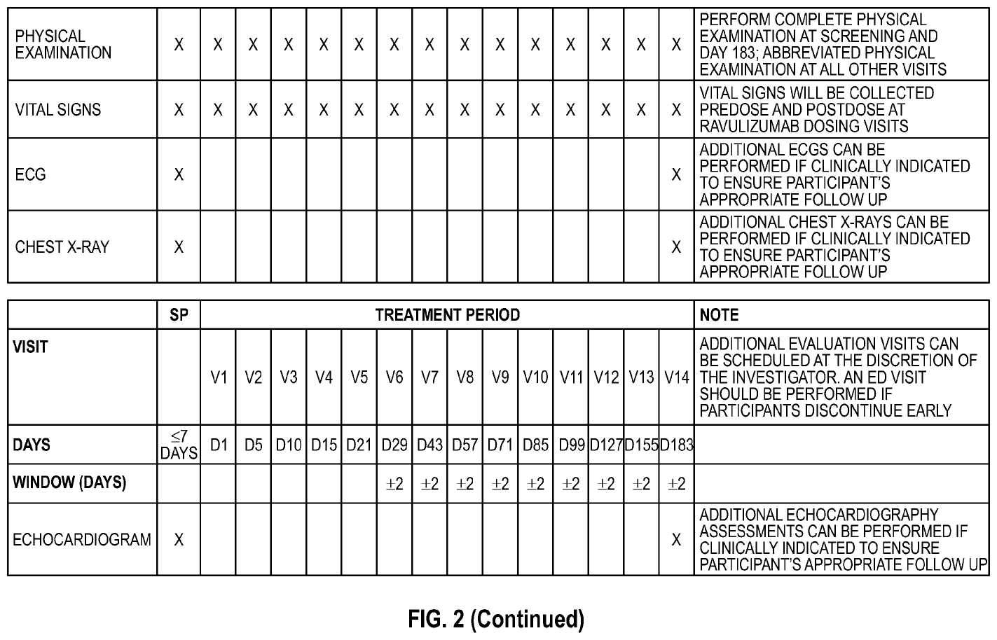

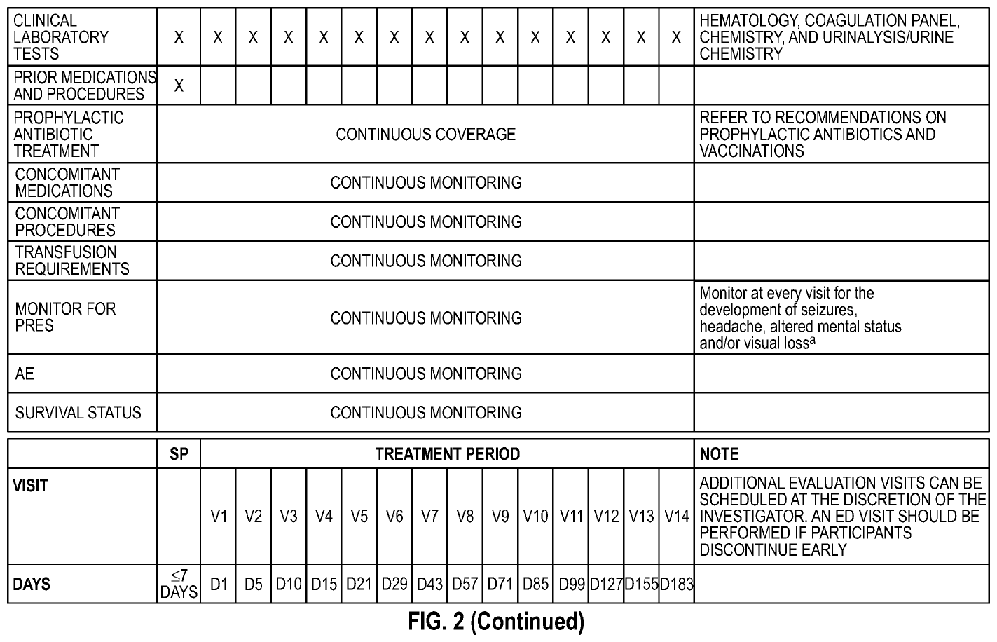

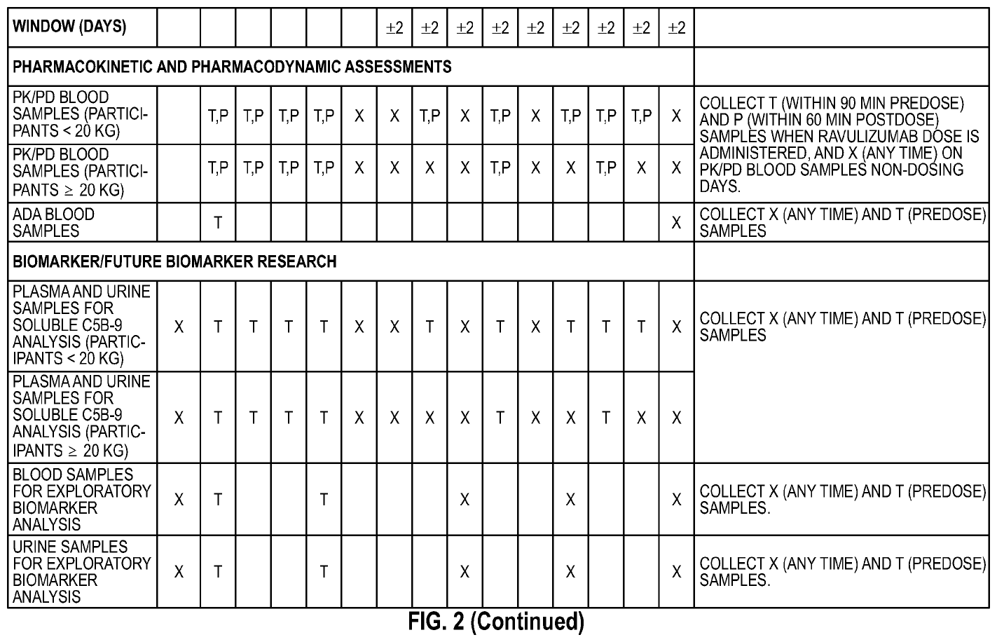

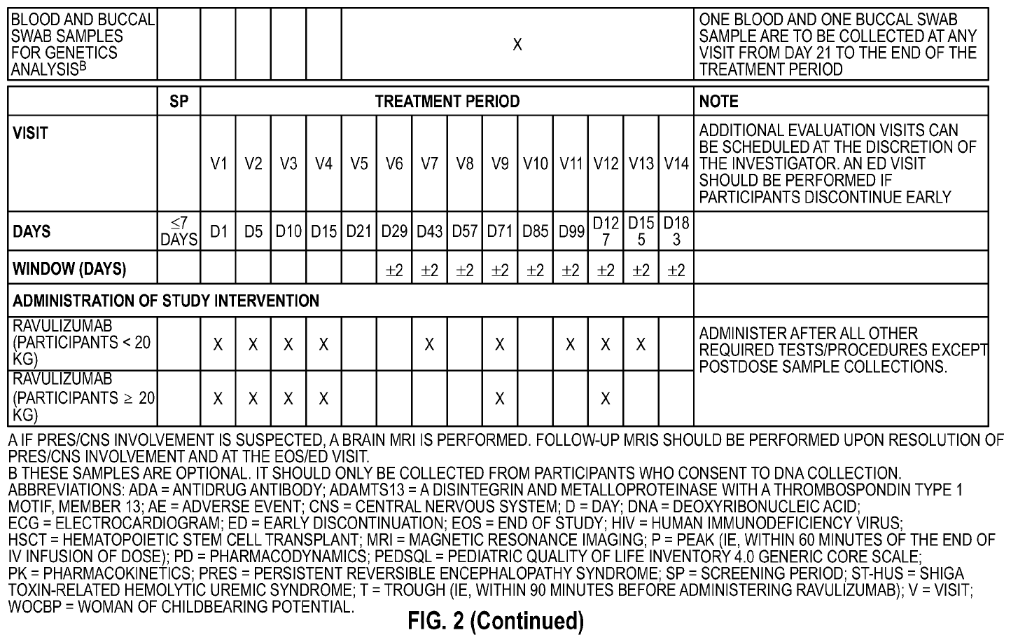

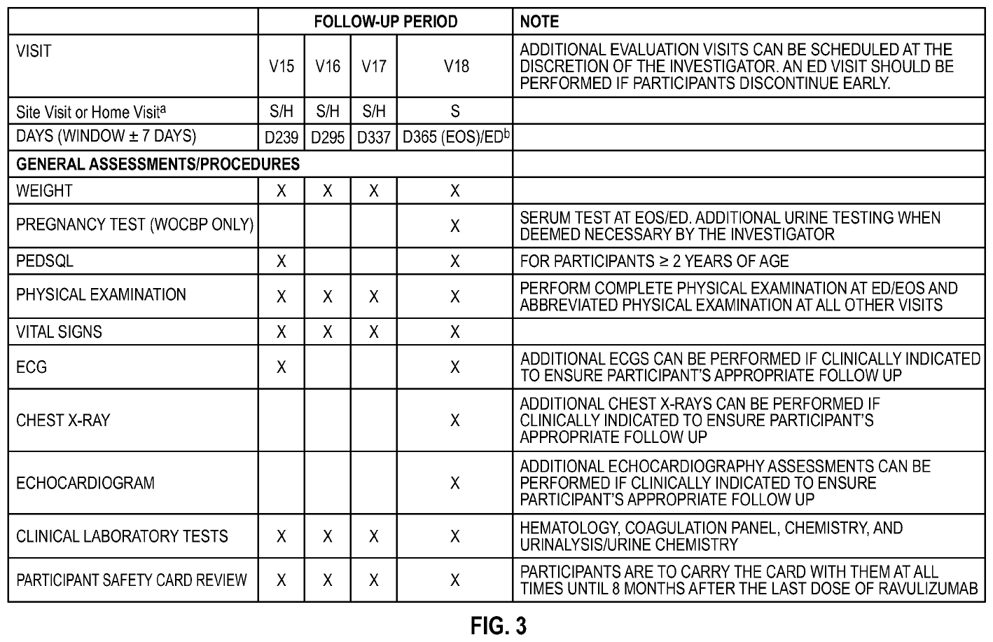

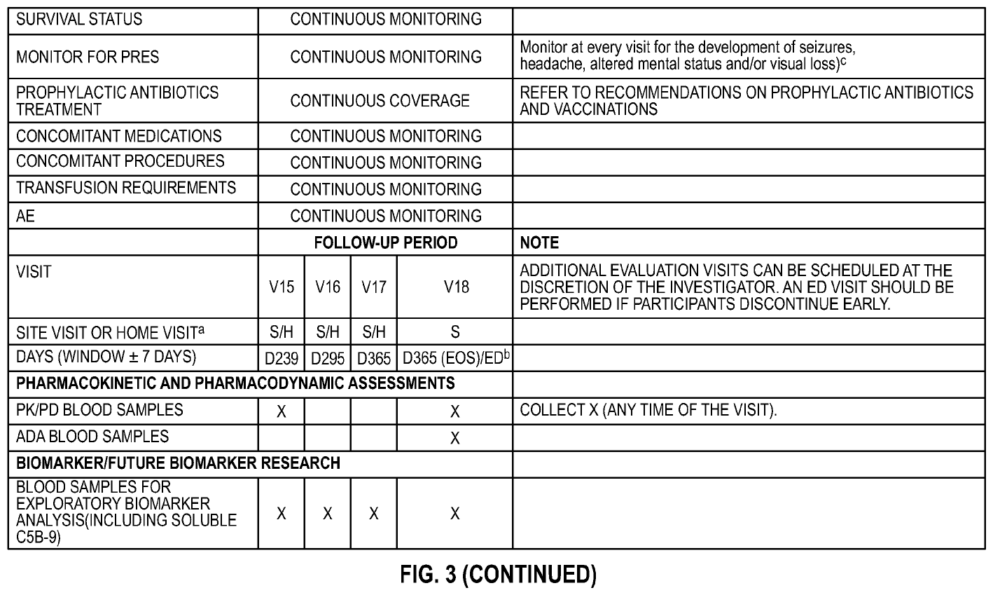

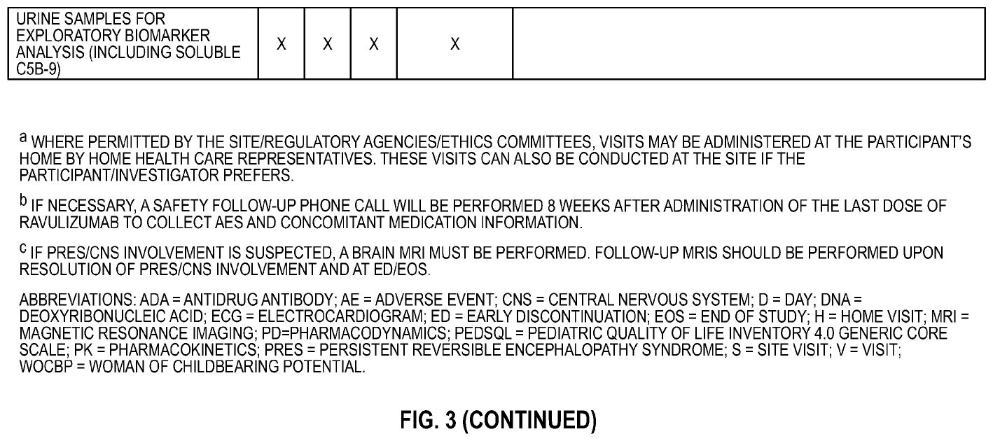

This study is an open-label, single arm, multicenter study to evaluate the safety, efficacy, PK, and PD of ravulizumab administered by intravenous (IV) infusion to pediatric participants, from 1 month to <18 years of age, with HSCT-TMA. An overview of the study is set forth in FIG. 1. The schedule of study visits and assessments through completion of the 26 week Treatment Period is presented in FIG. 2. The schedule of study visits and assessments for the Follow-up Period is presented in FIG. 3.

Participants with HSCT-TMA in whom the TMA persists for at least 72 hours after withdrawal or modification of any TMA triggering agent and/or treatment of any underlying trigger conditions are enrolled. All participants receive best supportive care (BSC) for the duration of the study. Approximately 40 participants are enrolled. The study consists of 3 periods: a Screening Period of up to 7 days, a 26-week Treatment Period, and a 26-week Follow-up Period.

Participants receive loading doses of ravulizumab IV on Day 1, Day 5 and Day 10 followed by maintenance dosing of ravulizumab IV on Day 15 and every 8 weeks (q8w) thereafter for participants weighing ≥20 kg, or once every 4 weeks (q4w) for participants weighing <20 kg. The first 10 treated participants receive the following weight-based ravulizumab dosing regimen via IV infusion as set forth in Table 1:

TABLE 1
Dosing Regimen

| | | Loading Phase Doses | | | | Maintenance Doses |
|---|---|---|---|---|---|---|
| Weight^{a} | | Day 1 | | Day 5 | Day 10 | Starting Day 15 |
| 5 to < 10 | kg | 600 | mg | 300 mg | 300 mg | 300 mg q4w |
| 10 to < 20 | kg | 600 | mg | 300 mg | 300 mg | 600 mg q4w |
| 20 to < 30 | kg | 900 | mg | 300 mg | 300 mg | 2100 mg q8w |
| 30 to < 40 | kg | 1200 | mg | 300 mg | 300 mg | 2700 mg q8w |
| 40 to < 60 | kg | 2400 | mg | 600 mg | 600 mg | 3000 mg q8w |
| 60 to < 100 | kg | 2700 | mg | 900 mg | 900 mg | 3300 mg q8w |
| ≥100 | kg | 3000 | mg | 900 mg | 900 mg | 3600 mg q8w |

^{a}Weight-based dosing is based on the participant's body weight recorded at the day of the infusion visit. If the weight at the day of the infusion can't be obtained, the weight recorded during the previous study visit may be used.

Supplemental doses of ravulizumab are administered following red blood cell (RBC) and/or platelet transfusions. In addition, supplemental doses of ravulizumab are allowed on an individual case basis for demonstrated clinical worsening.

After completion of the 26-week Treatment Period, all participants enter the Follow-up Period and remain in the study for 26 weeks without further ravulizumab administration. In the case of clinical need for extended treatment (for example, if a participant starts to exhibit TMA response late in the Treatment Period), the Investigator and the Medical Monitor may mutually agree on additional dosing into the Follow-up Period based on the weight-based dosing regimen.

If a participant meets criteria for TMA relapse or loss of TMA response during the Follow-up Period, the participant may receive ravulizumab retreatment.

For participants who withdraw from the study or participants who are administered ravulizumab during the Follow-up Period, a safety follow-up phone call is performed 8 weeks after the last dose of ravulizumab if 8 weeks after the last dose is later than the date of the End of Study (EoS) Visit or Early Discontinuation (ED) Visit.

### 3. Study Population

Approximately 40 treated pediatric participants with HSCT-TMA are enrolled in this study. Participants are enrolled and assigned to treatment with ravulizumab at approximately 50 investigative sites globally. At least 3 Japanese participants are included in the study.

a. Inclusion Criteria

Participants are eligible for enrollment in the study only if they satisfy all of the following criteria:
1. Participant must be 1 month of age up to <18 years of age at the time of signing the informed consent or assent form;
2. Pediatric participants who received HSCT within the past 6 months at the time of Screening;
3. A TMA diagnosis, based on all of the following criteria occurring simultaneously:
   De novo thrombocytopenia or platelet transfusion refractoriness, where: De novo thrombocytopenia is defined as new decline in platelet count to ≤50,000/mm3 and transfusion refractoriness is defined as failure of the platelet increment 10 to 60 minutes post transfusion to exceed the transfusion trigger, or a rise of less than 10×109/L 20 to 24 hours post-transfusion after at least 2 platelet transfusions
   De novo anemia (defined as a new decline in hemoglobin to ≤8 g/dL) or increase in transfusion requirements (defined as the need to administer more frequent transfusions in order to maintain hemoglobin 8 g/dL)
   Either one of the following markers of hemolysis: Lactate dehydrogenase ≥1.5×ULN or presence of schistocytes ≥2 high power field (HPF) in peripheral blood smear
   Proteinuria on spot urinalysis where proteinuria is defined as protein/creatinine ratio ≥2 mg/mg. The presence of proteinuria should be confirmed by a second measurement (with the 2 measurements at least 4 hours apart)
   Presence of hypertension defined as: Systolic blood pressure (SBP) and/or diastolic blood pressure (DBP), that is, on repeated measurement, ≥95th percentile for age, sex, and height;
4. Participants must have HSCT-TMA that persists for at least 72 hours after initial management of any triggering agent/condition (including withdrawal or dose reduction of the offending agent [e.g., CNIs]; treatment of any underlying infection; or treatment of underlying GVHD);
5. Body weight ≥5 kg at Screening;
6. Male or female [Contraceptive use by men or women should be consistent with local regulations regarding the methods of contraception for those participating in clinical studies. Male participants must agree to use contraception as detailed in the protocol during the Treatment Period and for at least 8 months after the final dose of ravulizumab and refrain from donating sperm during this period. A female participant is eligible to participate if she is not pregnant, not breastfeeding, and meets at least one of the following conditions: not a woman of childbearing potential (WOCBP) OR is a WOCBP and using a highly effective and acceptable contraceptive method during the Treatment Period and for at least 8 months after the final dose of ravulizumab. A WOCBP must have a negative serum pregnancy test at Screening and a negative urine pregnancy test before the first dose of ravulizumab];
7. Participants must be vaccinated against meningococcal infections if clinically feasible, according to institutional guidelines for immune reconstitution after HSCT. Participants must be re-vaccinated against Haemophilus influenzae type b (Hib) and Streptococcus pneumoniae if clinically feasible, according to institutional guidelines for immune reconstitution after HSCT. All participants are administered coverage with prophylactic antibiotics according to institutional posttransplant infection prophylaxis guidances including coverage against N. meningiditis for at least 2 weeks after meningococcal vaccination. Participants who cannot receive meningococcal vaccine, receive antibiotic prophylaxis coverage against N. meningiditis the entire Treatment Period and for 8 months following the final dose of ravulizumab; and
8. Participants or their legally authorized representative must be capable of giving signed informed consent or assent, which includes compliance with the requirements and restrictions listed in the informed consent or assent form and in this protocol.
   b. Exclusion Criteria

Participants are excluded from the study if any of the following criteria apply:
1. Known familial or acquired ‘a disintegrin and metalloproteinase with a thrombospondin type 1 motif, member 13’ (ADAMTS13) deficiency (activity <5%);
2. Known Shiga toxin-related hemolytic uremic syndrome (ST-HUS) as demonstrated by a positive test for Shiga toxin or culture of Shiga toxin producing bacteria;
3. Positive direct Coombs test;
4. Diagnosis or suspicion of disseminated intravascular coagulation (DIC) according to the International Society of Thrombosis and Haemostasis (ISTH) scoring criteria as set forth in Taylor et al., Thromb Haemost. 2001; 86(5):1327-1330 and Tables 2-3 [ISTH is used only in participants with clinical suspicion for DIC (e.g., excessive bleeding in the setting of malignancy, severe infection or sepsis, obstetric complications, trauma). For overt DIC, the participant has to have an underlying disorder known to be associated with overt DIC to use the algorithm];
5. Known bone marrow/graft failure;
6. Diagnosis of veno-occlusive disease (VOD) according to the European Society for Blood and Bone Marrow Transplantation (EBMT) criteria set forth in Corbacioglu S, et al., Bone Marrow Transplant. 2018; 53(2):138-145 and Table 4;
7. Human immunodeficiency virus (HIV) infection (evidenced by HIV-1 or HIV-2 antibody titer, or documented negative HIV-1/HIV-2 tests within 6 months prior to Screening);
8. Unresolved meningococcal disease;
9. Presence or suspicion of sepsis (treated or untreated) within 7 days prior to Screening;
10. Pregnancy or breastfeeding;
11. Hypersensitivity to murine proteins or to 1 of the excipients of ravulizumab;
12. Any medical or psychological condition that, in the opinion of the Investigator or Alexion, could increase the risk to the participant by participating in the study or confound the outcome of the study;
13. Previously or currently treated with a complement inhibitor; and
14. Participation in another interventional treatment study or use of any experimental therapy (except for the use of currently approved medications being investigated alone or in combination for: treatment of the underlying disease; conditioning regimen: GVHD prophylaxis treatment; infections prophylaxis; or posttransplant infection treatment) within 30 days before initiation of ravulizumab on Day 1 in this study or within 5 half-lives of that investigational product, whichever is greater.

TABLE 2
International Society of Thrombosis and
Haemostasis (ISTH) Scoring Criteria

| Variable | | Points |
|---|---|---|
| Platelet count, cells × 10⁹/L | ≥100 | 0 |
| | 50 to <100 | 1 |
| | <50 | 2 |
| Elevated levels of a fibrin-related marker^{a} | No increase | 0 |
| (eg, D-dimer, fibrin degradation products) | Moderate increase | 2 |
| | Severe increase | 3 |
| Prolonged PT, seconds | <3 | 0 |
| | 3 to <6 | 1 |
| | ≥6 | 2 |
| Fibrinogen level, g/L | ≥1 | 0 |
| | <1 | 1 |

^{a}Use central laboratory-specific cutoff values.

TABLE 3
International Society of Thrombosis
and Haemostasis (ISTH) Interpretation

| Score | Diagnosis |
|---|---|
| <5 | Not suggestive of overt DIC, may be non-overt DIC; repeat within |
| | next 1 to 2 days and manage clinically as appropriate |
| ≥5 | Compatible with overt DIC; treat for DIC as appropriate and repeat |
| | scoring daily |

TABLE 4
European Society for Blood and Bone Marrow Transplantation (EBMT)
Criteria for Diagnosis of Veno-Occlusive Disease (VOD)
Criteria

| | |
|---|---|
| 1. | No limitation for time of onset of SOS/VOD |
| 2. | The presence of 2 or more of the following (with the exclusion of |
| | other potential differential diagnoses): |
| | Unexplained consumptive and transfusion-refractory |
| | thrombocytopenia (≥1 weight-adjusted platelet substitution/day |
| | to maintain institutional transfusion guidelines). |
| | Otherwise unexplained weight gain on 3 consecutive days despite the |
| | use of diuretics, or a weight gain >5% above baseline value. |
| | Hepatomegaly (best if confirmed by imaging [suggested imaging: |
| | ultrasonography, computed tomography, or MRI]) above baseline |
| | value. |
| | Ascites (best if confirmed by imaging [suggested imaging: |
| | ultrasonography, computed tomography, or MRI]) above baseline |
| | value. |
| | Rising bilirubin from a baseline value on 3 consecutive days, or |
| | bilirubin ≥2 mg/dL within 72 hours. |

### 4. Study Intervention

Study intervention is defined as any investigational intervention(s), marketed product(s), placebo, or medical device(s) intended to be administered to a study participant according to the study protocol. Details regarding ravulizumab are set forth in Table 5.

TABLE 5
Ravulizumab

| | |
|---|---|
| Intervention Name | Ravulizumab |
| Type | Biologic |
| Dose Formulation | Vial |
| Unit Dose Strength(s) | 300 mg (10 mg/mL concentrated solution) |
| Dosage Level(s) | Weight-based dosing |
| Route of | IV infusion |
| Administration | |
| Use | Experimental |
| IMP or NIMP | IMP |
| Sourcing | Provided centrally by Alexion or contracted |
| | manufacturing organization |
| Packaging and | Ravulizumab will be provided in glass vials |
| Labeling | and stoppered with a butyl rubber stopper with |
| | aluminum overseal and flip-off cap. Ravulizumab |
| | will be supplied in kits and labeled as required |
| | per country requirement. |

Abbreviations: IMP = investigational medicinal product; IV = intravenous, NIMP = noninvestigational product.

In this study, all participants receive Best Supportive Care (BSC) as background therapy. BSC is determined by the Investigator according to institutional practices and participant characteristics. Best supportive care measures include but are not limited to transfusion support, Corticosteroids, Dialysis, and Antihypertensive medications.

Transfusion support is provided as required per institutional guidelines and based on the participant's clinical condition. In general, transfusion support is recommended as follows per the Joint United Kingdom (UK) Blood Transfusion and Tissue Transplantation Services Professional Advisory Committee (JAPC): (a) Red blood cell transfusion should be considered in participants with hemoglobin ≤7 g/dL or in participants with symptomatic anemia (e.g., dyspnea, and/or tachycardia) with hemoglobin ≤8 g/dL, (b) Prophylactic platelet transfusions should be given to participants as required with a transfusion trigger of 10×10⁹/L, and (c) an increase in the platelet transfusion threshold to 20×10⁹/L is justified in participants who are febrile and/or receiving antibiotic therapy for suspected bacterial or fungal infection. Transfusions of RBCs or platelets require supplemental doses of ravulizumab.

Supportive care for underlying conditions (e.g., HSCT, GVHD) is permitted during the course of the study. Use of currently approved medications being investigated alone or in combination for the treatment of the underlying disease, conditioning regimen, GVHD prevention regimen, GVHD treatment, infection prophylaxis, or infection treatment. Other concomitant medication may be considered on a case-by-case basis by the Investigator.

The following medicines and therapy are not allowed: (a) Eculizumab or other agents that act on the complement pathway, (b) use of plasma exchange, (c) use of rituximab, (d) IV immunoglobulin (Ig) (unless for an unrelated medical need; e.g., hypergammaglobulinemia), (d) Defibrotide (except for participants with confirmed VOD that develops during the study), and (e) the use of any experimental interventions or therapies.

The adequacy of the proposed dosing regimen is confirmed through initial analysis of PK/PD in the first 10 participants enrolled. If the proposed dose regimen does not result in the anticipated degree of free C5 inhibition, the dosing regimen is modified (increased) for subsequent participants according to PK/PD data.

Participants do not receive any additional treatment with ravulizumab as part of the protocol after completion of the study or withdrawal from the study. Upon completion of a participant's last study visit (i.e., EoS or ED Visit), the participant returns to the care of their treating physician.

### 5. Study Intervention

In rare instances, it may be necessary for a participant to permanently discontinue (definitive discontinuation) the study intervention. Participants should be considered for discontinuation from intervention if any of the following occur during the study: (a) Serious hypersensitivity reaction (b) Use of disallowed medication, (c) Pregnancy or planned pregnancy or (d) Investigator deems it is necessary for the participant. If the study intervention is definitively discontinued, every effort should be made to have the participant continue the study visits as per the Schedule of Activities for safety follow-up. If the participant does not agree to continue with the Schedule of Activities visits, the following activities should be completed: (1) early discontinuation assessments to be performed as indicated in the Schedule of Activities, (2) the site should follow the participant for at least 8 weeks after the final dose of ravulizumab via a safety phone call to collect any AEs and/or concomitant medications, and (3) information about survival status at 6 months and 1 year post first dose of ravulizumab should be collected. If a participant's survival status is not available at the time of discontinuation, sites attempt to collect the status (e.g., via public records, or telephone call).

A participant is considered lost to follow-up if he or she repeatedly fails to return for scheduled visits and is unable to be contacted by the study site.

### 6. Study Assessments and Procedures

A review of demographic parameters, including age, gender, race, and ethnicity is performed. The participant's relevant medical history, including prior and concomitant conditions/disorders (including TMA diagnosis), treatment history, and family history of relevant diseases is evaluated at the Screening Visit by the Investigator and documented in the source documents and CRF. Information collected includes: transplant modality, hematopoietic stem cell origin, transplant indication, history of prior HSCT, conditioning regimen, presence of GVHD (Section 10.11), engraftment status any other transplant complication(s), and any other relevant information on the peritransplant period.

At the scheduled dosing visits, ravulizumab IV administration is performed after all other tests and procedures have been completed, excluding the postdose sample collections (PK/PD/biomarkers). Weight-based dosing is based on the participant's body weight recorded at the day of the infusion visit. If the weight at the day of the infusion cannot be obtained, the weight recorded during the previous study visit can be used.

Ravulizumab is administered by IV infusion according to the dosing regimen in Table 1. In the case of clinical need for extended treatment (for example, if a participant starts to exhibit TMA response late in the Treatment Period), the Investigator and the Medical Monitor can mutually agree on additional dosing into the Follow-up Period based on the weight-based dosing regimen in Table 1. Unscheduled visits can be arranged as needed for ravulizumab administration. The following assessments must be performed at any unscheduled visit in which ravulizumab is administered: (a) A urine pregnancy test (WOCBP only), (b) Abbreviated physical examination, (c) PK/PD blood sample collection, (d) Vital signs, (e) Clinical laboratory tests, (f) C5b-9 blood sample collection, (g) C5b-9 urine sample collection.

Supplemental doses of ravulizumab are allowed during the Treatment Period according to the criteria outlined in Table 5.

### Table 6: Supplemental Dosing of Ravulizumab

TABLE 6
Supplemental Dosing of Ravulizumab

| Qualified Reason | Supplemental Dose of Ravulizumab |
|---|---|
| Participants who demonstrate defined clinical | For <2 weeks from the most recent |
| worsening (i.e., 2 or more of the following: | maintenance dose |
| doubling of serum creatinine from baseline, | Dose is determined following assessment |
| increase in LDH >25% compared to baseline, | and agreement by the Investigator and |
| reduction in platelets of >25% compared to | Medical Monitor |
| baseline, reduction in hemoglobin >10% | For ≥2 weeks after the most recent |
| compared to baseline, increased requirement | maintenance dose |
| for platelets or RBCs at any time) | A bodyweight-based maintenance dose |
| | can be given^{a} |
| Participants who receive RBC and/or platelet | Supplemental dose is administered the day |
| transfusions | after ≥1 RBC or platelet unit transfusions. |
| | Supplemental doses are not to be |
| | administered on regularly scheduled |
| | dosing days. |
| | Weight-based dosing as follows: |
| | 5 to <10 kg = 150 mg |
| | 10 to <40 kg = 300 mg |
| | ≥40 kg = 600 mg |
| | This dose is confirmed at the time of the |
| | dose confirmation analysis and adjusted |
| | subsequently if necessary. |

^{a}Following assessment and agreement by the Investigator and Alexion Medical Monitor

Unscheduled visits can be arranged as needed for supplemental ravulizumab administration. The following assessments must be performed during any unscheduled visit in which ravulizumab is administered: (a) Urine pregnancy test (WOCBP only), (b) Abbreviated physical examination, (c) PK/PD blood sample collection, (d) Vital signs, (e) Clinical laboratory tests, (f) C5b-9 blood sample collection, and (g) C5b-9 urine sample collection.

It is expected that participants are treated for the 26-week Treatment Period and then off treatment for the 26-week Follow-up Period. A participant may receive ravulizumab retreatment if he/she meets any of the following criteria during the Follow-up Period: (1) Participant meets criteria for TMA relapse, (2) Participant meets criteria for loss of TMA response, (3) The Investigator believes that the participant will potentially benefit from retreatment, initiates a discussion with the Medical Monitor, and provides a medically justified rationale. If retreatment is determined necessary, the participant continues to follow the Schedule of Activity as planned. The participant receives the ravulizumab weight-based dose according to the dosing regimen described in Table 1.

Unscheduled visits can be arranged as needed for ravulizumab administration. The following assessments must be performed during any unscheduled visit in which ravulizumab is administered: (a) Urine pregnancy test (WOCBP only), (b) Abbreviated physical examination, (c) PK/PD blood sample collection, (d) Vital signs, (e) Clinical laboratory tests, (f) C5b-9 blood sample collection, and (g) C5b-9 urine sample collection.

The number and volume of transfusions during the Screening Period are documented on the transfusion history CRF. The information collected includes date of the transfusion, number of units, and volume of each blood component given. Transfusions during the study are recorded on the transfusions CRF. The information collected includes date of the transfusion, number of units, and volume of each blood component given.

Due to its mechanism of action, the use of ravulizumab increases the participant's susceptibility to infection due to N. meningitidis. To reduce the risk of infection, participants are vaccinated against N. meningitidis if clinically feasible. Vaccines against serotypes A, C, Y, W135, and B, where available, are recommended to prevent common pathogenic meningococcal serotypes.

As HSCT results in immunosuppression due to ablative therapy, concomitant medication, or due to underlying disease, vaccine antibody titers will decrease 1 to 4 years after the transplant if the patient is not revaccinated. It is recommended that sites follow the relevant immune reconstitution guidelines or local practice guidelines for immune reconstitution for re vaccination of participants who have undergone HSCT.

Participants are administered prophylactic antibiotics for meningococcal infection until at least 2 weeks after vaccination. Vaccination may not be sufficient to prevent meningococcal infection. Consideration should be given per official guidance and local practice on the appropriate use of prophylactic antibacterial agents. All participants are monitored for early signs of meningococcal infection, evaluated immediately if infection is suspected, and treated with appropriate antibiotics, if necessary. Participants who cannot be vaccinated must receive antibiotic prophylaxis for the entire ravulizumab treatment period and for 8 months following the final dose of ravulizumab.

If clinically feasible, participants are also vaccinated against Hib and S. pneumoniae according to the appropriate immune reconstitution guidelines and participants who have undergone HSCT must be re-vaccinated. Participants who cannot be vaccinated must receive antibiotic prophylaxis for the entire ravulizumab treatment period and for 8 months following the final dose of ravulizumab.

### 7. Efficacy Assessments

Laboratory assessments to determine TMA response are performed according to the Schedule of Activities and assessed against predetermined criteria for TMA response. For time to TMA response, partial TMA response, loss of TMA response, and TMA relapse, the relevant clinical laboratory assessments are performed according to Table 7 and the Schedule of Activities and assessed against predetermined criteria for TMA response.

TABLE 7
Protocol-Required Safety Laboratory Assessments

| Laboratory | |
|---|---|
| Assessments | Parameters |
| Hematology | Platelet count |
| | Immature platelet fraction |
| | Red blood cell count |
| | Hemoglobin (including free hemoglobin) |
| | Hematocrit |
| | RBC Indices (mean corpuscular volume, mean |
| | corpuscular hemoglobin,) |
| | Percentage of reticulocytes |
| | RBC distribution width |
| | White blood cell count with differential (including early |
| | progenitors) |
| | Haptoglobin |
| | RBC morphology (peripheral blood smear); particularly the |
| | presence of schistocytes |
| Coagulation | INR |
| Panel | PT |
| | APTT |
| | D-Dimer |
| | Fibrinogen |
| Clinical | Liver function tests: |
| Chemistry^{a} | ALT |
| | AST |
| | ALP |
| | Albumin |
| | Total protein |
| | A/G ratio (albumin/globulin ratio) |
| | Bilirubin (total, direct and indirect) |
| | GGT |
| | Glucose (non-fasting) |
| | LDH |
| | Renal function: |
| | Anion gap |
| | Bicarbonate |
| | Blood urea nitrogen |
| | Calcium |
| | Chloride |
| | Creatinine |
| | Magnesium |
| | Phosphorus |
| | Potassium |
| | Sodium |
| | Uric acid |
| | Other clinical chemistry parameters: |
| | Amylase |
| | C-reactive protein |
| | Lipase |
| | Troponin I |
| | Von Willebrand Factor |
| Routine | Albumin |
| Urinalysis/ | Bilirubin |
| Urine | Blood |
| Chemistry | Color |
| | Creatinine |
| | Glucose |
| | Ketones |
| | Nitrite |
| | pH |
| | Protein |
| | Specific gravity |
| | Urobilinogen |
| Other | HIV antibody (HIV-1and HIV-2) |
| Study- | ADAMTS13 activity |
| specific | ST-HUS screen (eg, Shiga toxin enzyme immunoassay/PCR |
| Tests | in stool/stool culture/rectal swab) |
| | Coombs test, direct |
| | Serum or urine human chorionic gonadotropin pregnancy |
| | test (as needed for WOCBP)^{a} |
| | Immunogenicity (ADA) |
| | Serum PK |
| | Serum PD (total and free C5) |
| | Exploratory biomarkers |
| | Soluble C5b-9 |
| | Genetic testing (whole blood and buccal swab) |

^{a}Serum pregnancy test at Screening and End of Study Visit/Early Discontinuation Visit, and local urine pregnancy test at all other times as specified in SoA
Abbreviations: ADA = antidrug antibodies; ADAMTS13 = a disintegrin and metalloproteinase with a thrombospondin type 1 motif, member 13; ALP = alkaline phosphatase; ALT = alanine aminotransferase; APTT = activated partial thromboplastin time; AST = aspartate transaminase; C5 = complement component 5; GGT = gamma-glutamyltransferase; HIV = human immunodeficiency virus; INR = International normalized ratio; LDH = lactate dehydrogenase; PCR = polymerase chain reaction; PD = pharmacodynamics; PK = pharmacokinetics; PT = prothrombin time; RBC = red blood cell; SoA = Schedule(s) of Activities; ST-HUS = Shiga toxin-related hemolytic uremic syndrome; WOCBP = woman of childbearing potential.

Participants' survival status is collected continuously during the study. Information on any events of deaths and cause of death occurring during the study is collected.

Change from baseline in organ dysfunction (end organ involvement, i.e., renal system, cardiovascular system, CNS, pulmonary system, GI system) is described for participants. The parameters set forth in Table 8 are assessed at the timepoints specified in the Schedule of Activities.

TABLE 8
Clinical Parameters

Renal dysfunction: Kidney status is assessed by measuring protein/creatinine ratio, serum
creatinine, and calculating estimated glomerular filtration rate (eGFR). For participants who
require dialysis, change from baseline in dialysis requirements is recorded.
Cardiopulmonary involvement: Chest X-rays, electrocardiogram (ECG), and
echocardiography to assess the presence of signs of pulmonary or cardiovascular involvement
(including, but not limited to, pulmonary hypertension, pleural effusion, and pulmonary
edema) is performed. In addition, use of any ventilatory or respiratory support is reported in
the participant's CRF, along with associated oximetry.
Hypertension is assessed by blood pressure measurement at every visit as part of vital signs
collection, and analysis of concomitant medications to control the hypertension.
Central nervous system involvement: Participants are assessed at every visit for signs of
persistent reversible encephalopathy syndrome (PRES), including headache, confusion, visual
loss, and seizures. If PRES is suspected, confirmatory magnetic resonance imaging (MRI) is
be performed.
Gastrointestinal involvement: Participants are assessed for signs and symptoms of GI
involvement (e.g., diarrhea, vomiting, pain, and bleeding). The frequency and estimated
volume of GI bleeding will be recorded in the participant's CRF.

### 8. Safety Assessments

A complete physical examination includes, at a minimum, assessments of the following organs/body systems: skin, head, ears, eyes, nose, throat, neck, lymph nodes, chest, heart, abdomen, extremities, musculoskeletal, and neurological state. An abbreviated physical examination includes, at a minimum, a body-system relevant examination based upon Investigator judgment and participant symptoms. Investigators pay special attention to clinical signs related to previous serious illnesses. Weight and height are also be measured and recorded.

Additional physical examinations are performed as medically indicated during the study.

Oral temperature (° C. or ° F.), heart rate, respiratory rate, systolic and diastolic blood pressure (mm Hg), and pulse oximetry is assessed at Screening and at each subsequent study visit. Blood pressure and heart rate measurements are assessed with the participant in a seated position using a completely automated device. Manual techniques are used only if an automated device is not available.

Blood pressure and heart rate measurements are preceded by at least 5 minutes of rest for the participant in a quiet setting without distractions. Ideally, the same arm for each participant is used for measurements. 02 saturation (%) is collected using pulse oximetry. Vital signs are collected predose and postdose at ravulizumab dosing visits.

Single 12-lead ECGs are conducted to obtain heart rate, PR, QRS, QT, and QTc intervals (QT interval will be corrected for heart rate using Fridericia's formula [QTcF]). Participants must be supine for approximately 5 to 10 minutes before ECG collection and remain supine, but awake during ECG collection. The Investigator or Sub-Investigator are responsible for reviewing the ECG to assess whether the ECG is within normal limits and determine the clinical significance of the results. These assessments are recorded in the source documents and the CRF.

Both posteroanterior and lateral chest X-rays are obtained while the participant is at full inspiration at the timepoints specified in the SoA. The Investigator or designee Sub Investigator is responsible for reviewing the X-rays to assess the clinical significance of the results. These assessments are recorded in the source documents and the CRF. Particular attention should be paid to determine whether signs of pulmonary or cardiovascular involvement are present (including, but not limited to, pulmonary hypertension, pleural effusion, and pulmonary edema).

Transthoracic echocardiography is performed as per the Schedule of Activities. The Investigator or designee Sub-Investigator is responsible for reviewing the echocardiogram to assess the clinical significance of the results. These assessments are recorded in the source documents and the CRF. Particular attention should be paid to determine whether signs of pulmonary hypertension (right ventricular dysfunction, tricuspid regurgitation) or serositis (pericardial effusion) are present.

If a participant has symptoms of PRES at study entry or develops symptoms during the study, an MRI is performed to look for bilateral white matter abnormalities in the vascular watershed areas consistent with PRES. If the presence of PRES is confirmed, MRIs are performed upon resolution of symptoms and at the EoS/ED visit. Additional MRIs can be performed during the study.

All laboratory tests with values considered clinically significantly abnormal during participation in the study or within 8 weeks after the final dose of study intervention are repeated until the values return to normal or baseline or are no longer considered clinically significant by the Investigator or Medical Monitor. If such values do not return to normal/baseline within a period of time judged reasonable by the Investigator, the etiology should be identified. All protocol-required laboratory assessments are conducted in accordance with the laboratory manual and the Schedule of Activities. If laboratory values from non-protocol specified laboratory assessments performed at the institution's local laboratory require a change in participant management or are considered clinically significant by the Investigator (e.g., Serious Adverse Event (SAE) or Adverse Event (AE), or dose modification), then the results must be recorded in the source document.

Human immunodeficiency virus testing for HIV-1 and HIV-2 is required of all participants prior to enrollment. A documented history of negative HIV-1 and HIV-2 tests within 6 months prior to Screening is sufficient. Participants who are HIV positive are not enrolled.

Pregnancy testing is performed as described in the Schedule of Activities. Pregnancy data from female participants and female spouses/partners of male participants is collected and followed from the signing of the ICF/assent form until the outcome of the pregnancy is known, even if the participant discontinues study intervention or withdraws from the study. Any female participant who becomes pregnant while participating in the study is discontinued from the study intervention. Pregnancy is not considered as an AE unless there is a suspicion that the study intervention may have interfered with the effectiveness of a contraceptive medication. However, complications of pregnancy and abnormal outcomes of pregnancy are AEs and may meet the criteria for an SAE (e.g., ectopic pregnancy, spontaneous abortion, intrauterine fetal demise, neonatal death, or congenital anomaly). Elective abortions without complications should not be reported as AEs.

Prior medications and/or vaccines (including vitamins, herbal preparations, and those discussed in the exclusion criteria and procedures (any therapeutic intervention, such as surgery/biopsy or physical therapy) that the participant receives or undergoes within 30 days prior to first dose of ravulizumab on Day 1 are recorded.

Concomitant medications (including any medication, vitamin, herbal preparation or supplement) and procedures are those received after the start of ravulizumab IV infusion on Day 1. This includes any supportive care medications and procedures, antibiotic prophylaxis, GVHD prophylaxis or treatment, and vaccinations. At each study visit, participants are questioned about any new medication or nondrug therapies or changes to concomitant medications and nondrug therapies since the last visit. Any concomitant medication deemed necessary for the participant's care during the study, or for the treatment of any AE, along with any other medications, other than those listed as disallowed medications, can be given at the discretion of the Investigator.

Data regarding transfusion of blood components should be recorded in the CRF page, including date of transfusion, type of blood component administered, number of units, and volume.

### 9. Safety Assessments

An adverse event (AE) is any untoward medical occurrence in a participant, temporally associated with the use of study intervention, whether or not considered related to the study intervention. An AE can therefore be any unfavorable and unintended sign (including an abnormal laboratory finding), symptom, or disease (new or exacerbated) temporally associated with the use of study intervention. Events that meet the AE definition are set forth in Table 9. Events that do not meet the AE definition are set forth in Table 10.

TABLE 9
Events that Meet the AE Definition

Any abnormal laboratory test results (hematology, clinical chemistry, or urinalysis) or other
safety assessments (e.g., ECG, radiological scans, vital signs measurements), including those
that worsen from baseline, considered clinically significant in the medical and scientific
judgment of the Investigator (ie, not related to progression of underlying disease).
Exacerbation of a chronic or intermittent pre-existing condition including either an increase in
frequency and/or intensity of the condition.
New conditions detected or diagnosed after study intervention administration even though it
may have been present before the start of the study.
Signs, symptoms, or the clinical sequelae of a suspected drug-drug interaction.
Signs, symptoms, or the clinical sequelae of a suspected overdose of either study intervention
or a concomitant medication. Overdose per se is not reported as an AE/SAE unless it is an
intentional overdose taken with possible suicidal/self-harming intent. Such overdoses are
reported regardless of sequelae.
“Lack of efficacy” or “failure of expected pharmacological action” per se is not reported as an
AE or SAE. Such instances are captured in the efficacy assessments. However, the signs,
symptoms, and/or clinical sequelae resulting from lack of efficacy are reported as AE or SAE
if they fulfill the definition of an AE or SAE.

TABLE 10
Events that Do Not Meet the AE Definition

Medical or surgical procedure (e.g., endoscopy, appendectomy): The condition that leads to
the procedure is the AE. Situations in which an untoward medical occurrence did not occur
(e.g., hospitalization for elective surgery if planned before the signing the ICF, admissions for
social reasons or for convenience).
Anticipated day-to-day fluctuations of pre-existing disease(s) or condition(s) present or
detected at the start of the study that do not worsen.
A medication error (including intentional misuse, abuse, and overdose of the product) or use
other than what is defined in the protocol is not considered an AE unless there is an untoward
medical occurrence as a result of a medication error.
Cases of pregnancy that occur during maternal or paternal exposure to study intervention are
to be reported within 24 hours of Investigator/site awareness. Data on fetal outcome and
breastfeeding will be collected for regulatory reporting and safety evaluation.
Any clinically significant abnormal laboratory findings or other abnormal safety assessments
which are associated with the underlying disease, unless judged by the Investigator to be more
severe than expected for the participant's condition.
Any clinically significant abnormal laboratory findings or other abnormal safety assessments
which are associated with the underlying disease, unless judged by the Investigator to be more
severe than expected for the participant's condition.
The disease/disorder being studied or expected progression, signs, or symptoms of the
disease/disorder being studied, unless more severe than expected for the participant's
condition.
Situations in which an untoward medical occurrence did not occur (social and/or convenience
admission to a hospital).

If an event is not an AE per definition above, then it cannot be a Serious Adverse Event (SAE) even if serious conditions are met (e.g., hospitalization for signs/symptoms of the disease under study, death due to progression of disease). An SAE is defined as any untoward medical occurrence that, at any dose results in one of the events set forth in Table 11.

TABLE 11
SAE Definition

| | |
|---|---|
| 1. | Results in death |
| 2. | Is life-threatening |
| | The term “life-threatening” in the definition of “serious” refers to an event in which the |
| | participant was at risk of death at the time of the event. It does not refer to an event, which |
| | hypothetically might have caused death, if it was more severe. |
| 3. | Requires inpatient hospitalization or prolongation of existing hospitalization |
| | In general, hospitalization signifies that the participant has been detained (usually involving |
| | at least an overnight stay) at the hospital or emergency ward for observation and/or treatment |
| | that would not have been appropriate in the physician's office or outpatient setting. |
| | Complications that occur during hospitalization are AEs. If a complication prolongs |
| | hospitalization or fulfills any other serious criteria, the event is serious. When in doubt as to |
| | whether “hospitalization” occurred or was necessary, the AE should be considered serious. |
| | Hospitalization for elective treatment of a pre-existing condition that did not worsen from |
| | baseline is not considered an AE. |
| 4. | Results in persistent disability/incapacity |
| | The term disability means a substantial disruption of a person's ability to conduct normal |
| | life functions. |
| | This definition is not intended to include experiences of relatively minor medical |
| | significance such as uncomplicated headache, nausea, vomiting, diarrhea, influenza, and |
| | accidental trauma (e.g., sprained ankle) which may interfere with or prevent everyday |
| | life functions but do not constitute a substantial disruption. |
| 5. | Is a congenital anomaly/birth defect |
| 6. | Other situations: |
| | Medical or scientific judgment should be exercised in deciding whether SAE reporting is |
| | appropriate in other situations such as important medical events that may not be |
| | immediately life-threatening or result in death or hospitalization but may jeopardize the |
| | participant or may require medical or surgical intervention to prevent one of the other |
| | outcomes listed in the above definition. These events should usually be considered |
| | serious. |
| | Examples of such events include invasive or malignant cancers, intensive treatment in an |
| | emergency room or at home for allergic bronchospasm, blood dyscrasias or convulsions |
| | that do not result in hospitalization, or development of drug dependency or drug abuse. |

A suspected unexpected serious adverse reaction (SUSAR) is defined as a serious event that is not listed in the Investigator's Brochure and that the Investigator identifies as related to investigational product or procedure. United States Title 21 Code of Federal Regulations (CFR) 312.32 and European Union Clinical Trial Directive 2001/20/EC and the associated detailed guidances or national regulatory requirements in participating countries require the reporting of SUSARs. Suspected unexpected serious adverse reactions are reported to the national competent authority and IRBs/IECs where applicable.

All AEs are reported to the Investigator or qualified designee by the participant (or, when appropriate, by a caregiver, surrogate, or the participant's legally authorized representative). The Investigator and any qualified designees are responsible for detecting, documenting, and recording events that meet the definition of an AE or SAE and remain responsible for following up AEs that are serious, considered related to the study intervention or study procedures, or that caused the participant to discontinue the study intervention.

All AEs and SAEs are collected from the signing of the ICF/Assent Form until the ED/EoS Visit or 8 weeks after the final dose of ravulizumab (whichever occurs later). All SAEs are recorded and reported immediately and under no circumstance should this exceed 24 hours. Investigators are not obligated to actively seek AE or SAE data after conclusion of the study participation.

After the initial AE/SAE report, the Investigator is required to proactively follow-up on each participant at subsequent visits/contacts. All SAEs are followed up until resolution, stabilization, the event is otherwise explained, or the participant is lost to follow-up. For this study, any dose of ravulizumab greater than that specified in the protocol will be considered an overdose.

Overdoses are medication errors that are not considered AEs unless there is an untoward medical occurrence resulting from the overdose.

For recording and follow-up of an AE and/or SAE, the intensity is assessed. An event is defined as “serious” when it meets at least one of the predefined outcomes as described in the definition of an SAE, not when it is rated as severe. The Investigator makes an assessment of intensity for each AE and SAE reported during the study and assign it to one of the following categories from National Cancer Institute CTCAE v5.0, published 27 Nov. 2017: (a) Grade 1: Mild (awareness of sign or symptom, but easily tolerated), (b) Grade 2: Moderate (discomfort sufficient to cause interference with normal activities), (c) Grade 3: Severe (incapacitating, with inability to perform normal activities), (d) Grade 4: Life-threatening, or (e) Grade 5: Fatal.

The Investigator is obligated to assess the relationship between the study intervention and each occurrence of each AE or SAE. An Investigator causality assessment must be provided for all AEs (both nonserious and serious). This assessment must be recorded in the CRF and on any additional forms, as appropriate. The definitions for the causality assessments are as follows:
(a) Not related: There is no reasonable possibility the study intervention caused the AE. The AE has a more likely alternative etiology; it may be due to underlying or concurrent illness, complications, concurrent treatments, or effects of another concurrent drug. The event does not follow a reasonable temporal relationship to administration of the study intervention
(b) Related: There is a reasonable possibility the study intervention caused the AE. The AE has a temporal relationship to the administration of the study intervention. The event does not have a likely alternative etiology. The event corresponds with the known pharmaceutical profile of the study intervention. There is improvement on discontinuation and/or reappearance on rechallenge.

The Investigator uses clinical judgment to determine the relationship. Alternative causes, such as underlying disease(s), concomitant therapy, and other risk factors, as well as the temporal relationship of the event to study intervention administration are considered and investigated. The Investigator also consults the Investigator's Brochure (IB) and/or Product Information, for marketed products, in his/her assessment. For each AE/SAE, the Investigator must document in the medical notes that he/she has reviewed the AE/SAE and has provided an assessment of causality. There may be situations in which an SAE has occurred, and the Investigator has minimal information to include in the initial report to Alexion. However, it is very important that the Investigator always make an assessment of causality for every event before the initial transmission of the SAE data to Alexion. The Investigator may change his/her opinion of causality in light of follow-up information and send an SAE follow-up report with the updated causality assessment. The causality assessment is one of the criteria used when determining regulatory reporting requirements.

Blood samples are collected for determination of serum ravulizumab, free C5, and total C5 concentrations at the timepoints specified in the Schedule of Activities. The timing of sampling can be altered during the course of the study, based on newly available data (e.g., to obtain data closer to the time of peak plasma concentrations) to ensure appropriate monitoring. Samples collected for PK/PD analyses can also be used to evaluate safety or efficacy aspects related to concerns arising during or after the study.

For all participants receiving ravulizumab: Predose PK and PD blood samples are collected within 90 minutes before administering ravulizumab at visits specified in the Schedule of Activities. The predose blood sample can drawn through the venous access created for the dose infusion, prior to administration of the dose. Postdose PK and PD blood samples are collected within 60 minutes after completing ravulizumab infusion. The postdose blood samples are drawn from the participant's opposite, non-infused arm. PK/PD blood samples at a non-dosing visit can be collected at any time during the visit. In the event of an unscheduled visit, PK and PD blood samples are collected as soon as possible. Predose and postdose PK and PD samples are collected for all supplemental doses of ravulizumab administered. In the case of a participant increasing in weight from <20 kg (ravulizumab administration q4w) to ≥20 kg (ravulizumab administration q8w), the change in the dose interval from q4w to q8w only happens on Day 15, 71, or 127.

For participants who sign an additional optional consent, whole blood and buccal swab samples for exploratory genetics may/will be collected at the time point specified in Schedule of Activities. Exploratory genetics can be performed to investigate genetic variants in genes that may be associated with complement dysregulation or metabolism or efficacy of ravulizumab.

Blood and urine samples for biomarker research are collected from all participants at the time points specified in the Schedule of Activities. Whenever possible, collection of the biomarker samples during the study, including samples collected during the Screening Period, is performed prior to the participant receiving transfusions or dialysis. Biomarkers are measured and include, but are not limited to, assessments of the following: (1) Vascular inflammation (e.g., shed tumor necrosis factor receptor 1 [TNF-R1]), (2) Endothelial damage and/or activation (e.g., thrombomodulin and shed vascular cell adhesion molecule 1 [VCAM-1]), (3) Renal injury (e.g., Cystatin C), and (4) complement proteins and complement activation pathway products.

Antidrug antibodies to ravulizumab are evaluated in serum samples collected predose (within 90 minutes prior to the start of ravulizumab IV infusion) from all participants at time points specified in the Schedule of Assessments. Additionally, serum samples should also be collected at the final visit from participants who discontinued ravulizumab or were withdrawn from the study. Serum samples are screened for antibodies binding to ravulizumab and the titer of confirmed positive samples will be reported. Other analyses can be performed to further characterize the immunogenicity of ravulizumab.

The detection and characterization of antibodies to ravulizumab is performed using a validated assay method. Samples collected for detection of antibodies to ravulizumab are also evaluated for ravulizumab serum concentration to enable interpretation of the antibody data. Confirmed antibody positive samples are further evaluated for antibody titer and the presence of neutralizing antibodies.

The Pediatric Quality of Life Inventory (PedsQL) 4.0 Generic Core Scales are multidimensional child self-reports and parent proxy-report standardized instruments to measure health related quality of life (QoL) in children and adolescents 2-18 years of age. Sample PedsQL are set forth in FIGS. 4-10. The PedsQL are administered prior to performing other study procedures at a study visit (participants ≥2 years of age only).

### 10. Statistical Considerations

Approximately 40 participants are enrolled in this study. This sample size is deemed appropriate to provide complete safety information and the necessary precision level for the planned estimation. Assuming a proportion of participants achieving TMA response of 50%, 40 participants would yield a 95% CI for the proportion of response with a half-width of approximately 16%. The analysis sets are defined in Table 12.

TABLE 12
Analysis Sets

| Analysis set | Description |
|---|---|
| Enrolled Set | All consented participants meeting eligibility |
| | criteria. |
| Safety Set | All participants who receive at least 1 dose of |
| | ravulizumab. |
| Full Analysis Set | All participants who receive at least 1 dose of |
| | ravulizumab. |
| Pharmacokinetic | All participants who receive at least 1 dose of |
| and | ravulizumab and who have evaluable |
| Pharmacodynamic | pharmacokinetic or pharmacodynamic data. |
| Analysis Set | |
| Per Protocol Set | Will be defined in the statistical analysis plan. |

Efficacy analyses are performed on the Full Analysis Set (FAS). The primary efficacy endpoint analysis, as well as selected secondary endpoint analyses, are also performed on the Per Protocol Set. Safety analyses are performed on the Safety Set. Pharmacokinetic and PD analyses are performed on all participants who receive at least 1 dose of ravulizumab and who have evaluable PK and/or PD data.

Statistical methods described in this section are further elaborated in a separate statistical analysis plan (SAP). Summary statistics re computed and displayed by visit, where applicable, and by stratification factors, when relevant. Descriptive statistics for continuous variables minimally include the number of participants, mean, standard deviation, minimum, median, and maximum. For categorical variables, frequencies, and percentages are presented. Graphical displays are provided as appropriate. Analyses is performed using the SAS@software Version 9.4 or higher.

Analyses of the primary endpoint is performed by subgroups including, but not limited to the presence of GVHD, geographical region, and baseline weight. Further details are provided in the SAP.

The primary efficacy endpoint is TMA response during the 26-week Treatment Period. Participants must meet each TMA response criterion at 2 separate assessments obtained at least 28 days apart, and any measurement in between; and all intervals during which each criterion is met must overlap for at least one day to be classified as having met the primary efficacy endpoint.

The primary analysis consists of estimating the proportion of TMA responders among ravulizumab treated participants. This is performed by calculating the point estimate and a 95% CI for the proportion of TMA responders in ravulizumab treated participants. The CI is based on exact confidence limits using the Clopper-Pearson method.

For time to TMA response, participants are assigned as responders at the time of their TMA response and censored at their discontinuation time, or at the end of available follow-up if they have not responded by then. Cumulative incidence of TMA response is estimated using competing risk survival analysis methods to account for death as a competing risk. Point estimates and 95% CIs are provided.

In addition to the primary analysis of TMA Response during the 26-week Treatment Period, TMA response is also summarized over time by presenting the number and proportion of responders along with a 2-sided 95% CI for each postbaseline time point. This analysis is conducted using 2 different approaches. The first one presents the number and proportion of responders at a specific time point out of the participants who are still in the study up to this time point. The second approach presents the cumulative number and proportion of responders up to a specific time point out of all participants. This analysis includes time points of importance such as Week 26 and Week 52.

Response to each individual component of TMA response) during the 26 week Treatment Period is summarized by calculating the point estimate and a 95% CI for the proportion of responders. The CI is based on exact confidence limits using the Clopper Pearson method. These responses are also summarized over time by presenting the number and proportion of responders along with a 2-sided 95% CI for each postbaseline time point. This analysis is conducted using 2 different approaches. The first one presents the number and proportion of responders at a specific time point out of the participants who are still in the study up to this time point. The second approach presents the cumulative number and proportion of responders up to a specific time point out of all participants.

Time to response for each criterion of TMA response is assessed separately. Participants are assigned as responders for a specific criterion at the time of their response for that criterion and are censored at the time of their discontinuation or at the end of available follow-up if they have not responded by then.

Cumulative incidence of the response criterion of interest is estimated using competing risk survival analysis methods to account for death as a competing risk. Point estimates and 95% CIs are provided.

For hematologic response, participants must meet each response criterion at 2 separate assessments obtained at least 28 days apart, and any measurement in between; and all intervals during which each criterion is met must overlap for at least one day to be classified as having met this efficacy endpoint. Hematologic response during the 26-week Treatment Period is summarized by calculating the point estimate and a 95% CI for the proportion of responders. The CI is based on exact confidence limits using the Clopper-Pearson method. Response is also summarized over time by presenting the number and proportion of responders along with a 2-sided 95% CI for each postbaseline time point. This analysis is conducted using 2 different approaches. The first one presents the number and proportion of responders at a specific time point out of the participants who are still in the study up to this time point. The second approach presents the cumulative number and proportion of responders up to a specific time point out of all participants.

For time to hematologic response, participants are assigned as responders at the time of their response and are censored at their discontinuation time or at the end of available follow-up if they have not responded by then. Cumulative incidence of hematologic response is estimated using competing risk survival analysis methods to account for death as a competing risk. Point estimates and 95% CIs are provided.

The following criterion must be met for hemoglobin response: Hemoglobin ≥8 g/dL without the need for RBC transfusions. Participants must meet this criterion at 2 separate assessments obtained at least 28 days apart, and any measurement in between, and they should not be receiving RBC transfusion during this period.

Hemoglobin maintenance during the 26-week Treatment Period is summarized by calculating the point estimate and a 95% CI for the proportion of responders. The CI is based on exact confidence limits using the Clopper-Pearson method.

Response is also summarized over time by presenting the number and proportion of responders along with a 2-sided 95% CI for each postbaseline time point. This analysis is conducted using 2 different approaches. The first one presents the number and proportion of responders at a specific time point out of the participants who are still in the study up to this time point. The second approach presents the cumulative number and proportion of responders up to a specific time point out of all participants.

Hematologic parameters (hemoglobin, platelets, LDH, and schistocytes) are summarized by at baseline and each postbaseline time point using descriptive statistics for continuous variables for the observed value, as well as the change from baseline.

Partial TMA response (participant meets ≥1, but not all, criteria for TMA response during the 26-week Treatment Period is summarized by calculating the point estimate and a 95% CI for the proportion of responders. The CI is based on exact confidence limits using the Clopper-Pearson method. This includes separate presentations for participants with the following: 1) response to only 1 component; 2) response to at least 1, but not all components; and 3) response to at least 2, but not all components.

Partial TMA response are also summarized over time by presenting the number and proportion of responders along with a 2-sided 95% CI for each postbaseline time point.

Treatment effect on organ dysfunction is evaluated in the following end organ systems: renal, cardiovascular, CNS, pulmonary, and GI.

Kidney function parameters (protein/creatinine ratio, serum creatinine, and eGFR) are be summarized at baseline and each postbaseline time point using descriptive statistics for continuous variables for the observed value as well as the change from baseline. For participants who require dialysis, change from baseline in dialysis requirements is summarized. An analysis presents the number and proportion of participants who require dialysis over time. A 2-sided 95% CI for the proportion is provided. A participant is considered as not requiring dialysis at a specific postbaseline time point if they have been dialysis free for at least 5 days prior to that time point.

The presence of pulmonary hypertension, pleural effusion, pulmonary edema, and pericardial effusion is summarized. An analysis presents over time the number and proportion of participants with any of these conditions. A 2-sided 95% CI for the proportion is provided. Additionally, use of any ventilatory or respiratory support and changes over time is summarized.

Hypertension is assessed by blood pressure measurement at every visit as part of vital signs collection, and assessment according to percentile tables for age, sex, and height as well as with assessment of the use any concomitant medications to control the hypertension. Presence of hypertension is summarized by presenting the number and proportion of participants who shift in their hypertensive status from baseline to post baseline visits. Selected echocardiography parameters is summarized over time.

The presence of persistent reversible encephalopathy syndrome (PRES) is summarized by presenting the number and proportion of participants with shifts in presence of PRES status from baseline to post baseline visits. Signs and symptoms of GI involvement such as: diarrhea, vomiting, pain, and bleeding will be summarized. An analysis presents over time the number and proportion of participants with any of these conditions.

The frequency and estimated volume of GI bleeding is also summarized.

Among participants who will have achieved TMA response, TMA relapse (participant meets the criteria for TMA diagnosis after achieving TMA response) is summarized by calculating the point estimate and a 95% CI for the proportion of participants with a TMA relapse. The CI is based on exact confidence limits using the Clopper-Pearson method. An analysis summarizes time to TMA relapse from the time of response. Participants are censored at the time of the end of their available follow-up if they have not experienced a relapse. A Kaplan Meier cumulative distribution curve is generated. A corresponding summary table present the first quartile, median, and third quartile, along with corresponding 2-sided 95% CI, of time to TMA relapse.

Loss of response occurs when a participant achieves TMA response and fails to meet the criteria for one or more components of TMA response at a subsequent visit (must be confirmed by a second laboratory result at least 24 hours apart). Participants who achieve loss of response are summarized by calculating the point estimate and a 95% CI for the proportion of participants with a loss of TMA response. The CI re based on exact confidence limits using the Clopper-Pearson method. Duration of response is assessed with an analysis of time to loss of TMA response from the time of response. Participants are censored at the time of the end of their available follow-up if they have not experienced a loss of response. A Kaplan Meier cumulative distribution curve is generated. A corresponding summary table presents the first quartile, median, and third quartile, along with corresponding 2-sided 95% CI, of time to loss of TMA response.

Survival time is assessed as the number of days from the start of treatment to an event of death. Participants who survive are censored at their last available follow-up. A Kaplan Meier cumulative distribution curve is generated. A corresponding summary table present the first quartile, median, and third quartile, along with corresponding 2-sided 95% CI, of survival time.

Non-relapse mortality is defined as participant's death due to any cause during the study, with the exception of death due to underlying disease progression or relapse. Cumulative incidence of non-relapse deaths is estimated using competing risk survival analysis methods to account for relapse-related deaths as a competing risk. Point estimates and 95% CIs are provided. Subjects who do not experience anon-relapse death are right censored at their last available follow-up.

The following criterion must be met for platelet response: Platelet count ≥100,000/mm3 without transfusion support during the prior 7 days. Participants must meet this criterion at 2 separate assessments obtained at least 28 days apart, and any measurement in between, and they should not be receiving transfusion during this period. Maintenance of platelet count ≥100,000/mm3 during the 26-week Treatment Period is summarized by calculating the point estimate and a 95% CI for the proportion of responders. The CI is based on exact confidence limits using the Clopper Pearson method.

Response is also summarized over time by presenting the number and proportion of responders along with a 2-sided 95% CI for each postbaseline time point. This analysis is conducted using 2 different approaches. The first one presents the number and proportion of responders at a specific time point out of the participants who are still in the study up to this time point. The second approach presents the cumulative number and proportion of responders up to a specific time point out of all participants enrolled.

This being an estimation study, no multiplicity adjustment is implemented.

All safety analyses are performed on the Safety Set.

The definitions for AEs are set forth in Table 13.

TABLE 13
AE Definitions

Pretreatment AEs: Any AE that starts after providing informed consent/
assent, but before infusion of ravulizumab.
Pretreatment AEs: Any AE that starts after providing informed consent/
assent, but before the first infusion of ravulizumab.
Pretreatment AEs: Any AE that starts after providing informed consent/
assent, but before the first infusion of ravulizumab. Treatment-emergent
AE (TEAE): Any AE that starts during or after the start of the first
infusion of ravulizumab.
Treatment-emergent SAE (TESAE): A TEAE that is serious.

The incidence of TEAEs, TEAEs leading to withdrawal from the study, TEAEs leading to study treatment discontinuation, drug-related TEAEs, TEAEs during ravulizumab administration, and SAEs is summarized. All AEs are coded using the Medical Dictionary for Regulatory Activities (MedDRA) Version 22 or higher and are summarized by System Organ Class (SOC) and Preferred Term overall, by severity, and by relationship to treatment. Detailed by participant listings of TEAEs, SAEs, related TEAEs, TEAEs during ravulizumab administration, TEAEs leading to withdrawal from the study, and TEAEs leading to study treatment discontinuation are provided.

Clinically significant adverse changes from baseline in physical examination findings will be classified as AEs and analyzed accordingly. Vital signs are summarized descriptively at baseline and postbaseline time points and for changes from baseline.

Observed values and changes from baseline in clinical chemistry, hematology, and urinalysis are summarized descriptively at baseline and at each postbaseline time point. For laboratory results that can be classified as normal, low or high based on normal range values, shifts from baseline in classification are summarized for all study visits.

By-participant data listings of ECG parameters are provided. Electrocardiograms are be evaluated and summarized as normal, abnormal not clinically significant, or abnormal clinically significant. A shift from baseline to worst on-study ECG table is presented for ECG results. Observed values and change from baseline in ECG intervals (PR, RR, QT, and QTc) are summarized descriptively at baseline and each postbaseline time point. QT interval is corrected for heart rate using QTcF.

The incidence and titers for ADAs to ravulizumab is presented at each postbaseline time point in tabular format. Additionally, any confirmed ADA positive samples are tested for antibody titer and the presence of neutralizing antibodies to ravulizumab.

Individual PK/PD data is collected for all participants.

Graphs of mean serum ravulizumab concentration-time profiles are constructed. Graphs of serum concentration-time profiles for individual participants can also be provided. Actual dose administration and sampling times are used for all calculations. Descriptive statistics are calculated for serum concentration data at each sampling time, as appropriate. The PD effects of ravulizumab are evaluated by assessing the absolute values and changes and percentage changes from baseline in serum free C5 and serum total C5 concentrations over time, as appropriate. Descriptive statistics are calculated for the PD data at each sampling time, as appropriate.

For exploratory biomarker analyses, summary statistics are presented for observed values, change and percentage change from baseline. The relationship between ravulizumab concentration and exploratory biomarkers or the correlation between clinical benefit and key exploratory biomarkers are assessed by graphical display. Exploratory analysis and potential relationships between clinical outcomes, PK/PD, genetic profile, and biomarker levels can be performed and results summarized if evaluated.

Quality of life is evaluated using PedsQL for participants ≥2 years of age. The scales (Physical Functioning, Emotional Functioning, Social Functioning and School Functioning), summary scores (Psychosocial Health and Physical Health) and Total Scale Score are calculated based on the instrument instruction. Treatment of missing items also follow the instructions: when more than half of the items from any scale are missing, the scale score is not be computed.

The scales, summary scores, and total scale score are summarized at baseline and each postbaseline time point using descriptive statistics for continuous variables for the observed value as well as the change from baseline.

Hospitalizations are summarized in a table presenting the number and proportion of participants requiring hospitalization, as well as the number of hospitalizations, the duration, and the reason for hospitalization. In addition, the number and proportion of participants requiring readmission due to TMA and the number of readmissions due to TMA is summarized.

Transfusion requirements are summarized by presenting the number and proportion of participants requiring transfusions of RBCs and/or platelets, as well as the number of transfusions.

Outpatient medical encounters are summarized by presenting the number and proportion of participants requiring physician or emergency room visits and the underlying reason.

Exploratory genetics can be performed to investigate genetic variants in genes known to be associated with HSCT TMA, as well as to identify novel genetic variants associated with HSCT TMA, complement dysregulation, or metabolism or efficacy of ravulizumab.

An early analysis of the PK/PD data is initiated when the 10th participant completes Visit 5 on Day 21. All PK and free C5 data collected in all participants by that time are analyzed to confirm the adequacy of the initial dosing regimen to achieve complete inhibition of C5. If necessary, the dosing regimen is adjusted.

A primary endpoint analysis is conducted at the interim time point when all participants have been followed for 26 week Treatment Period from start of study treatment or withdraw from the initial 26 weeks Treatment Period. The SAP describes the planned analysis in greater detail. The final study analysis is conducted at the end of the study.

### Example 2: A Phase 3, Open-Label, Randomized, Multicenter Study of Ravulizumab in Adult and Adolescent Participants Who have Thrombotic Microangiopathy (TMA) after Hematopoietic Stem Cell Transplant (HSCT)

A two-stage open-label, randomized, multicenter study of ravulizumab in adolescent (12 to 17 years of age) and adult participants with HSCT-TMA is conducted.

### 1. Objectives

The primary objective of the study is to assess the efficacy of ravulizumab plus BSC versus BSC-only in the treatment of adult and adolescent participants with HSCT-TMA via patient TMA response. TMA response during the 26-week randomized Treatment Period, where TMA response is defined as meeting all of the following: (a) platelet count ≥50,000/mm3 without transfusion support during the prior 7 days, (b) LDH<1.5×ULN and absence of schistocytes (if there were schistocytes present at baseline) and (c) at least 50% reduction of proteinuria from baseline. Proteinuria is defined as protein/creatinine ratio ≥0.5 mg/mg. Participants must meet each response criterion at 2 separate assessments obtained at least 28 days apart, and any measurement in between; and all intervals during which each criterion is met must overlap for at least one day.

A secondary objective includes characterizing TMA response after treatment with ravulizumab, as assessed by: (a) time to TMA response measured in days from Day 1 to the first day that the participant satisfies all the criteria for TMA response), (b) TMA response at Week 26 and Week 52, (c) response and time to response for each individual component of TMA response during the 26 week randomized Treatment Period, (d) Hematologic response a defined as: platelet count ≥50,000/mm³ without transfusion support during the prior 7 days and LDH<1.5×ULN and absence of schistocytes (if there were schistocytes present at baseline), (e) time to hematologic response, (f) hemoglobin response (ability to maintain hemoglobin ≥8 g/dL without transfusion support, (g) partial response (participant meets ≥1, but not all, criteria for TMA response), and (h) loss of TMA response (participant achieves TMA response and at a subsequent visit fails to meet the criteria for one or more components of TMA response, with confirmation by a second laboratory result at least 24 hours apart).

Additional secondary objectives include assessing: (a) improvement in organ dysfunction (change from baseline in TMA-associated organ dysfunction in renal system, cardiovascular system, pulmonary system, CNS, and GI system at 6 months and 1 year), (b) TMA relapse and duration of response (TMA relapse during the study (participant meets the criteria for TMA diagnosis after achieving TMA response) and duration of TMA response), (c) overall survival (at 6 months and 1 year), (d) non-relapse mortality (defined as participant's death due to any cause during the study, with the exception of death due to underlying disease progression or relapse), and (e) platelet response (platelet response defined as platelet count ≥100,000/mm3 without transfusion support during the prior 7 days). Criterion must be met at 2 separate assessments obtained at least 28 days apart, and any measurement in between.

Pharmacokinetic and Pharmacodynamic endpoints include assessing PK/PD of ravulizumab in adult and adolescent participants with HSCT-TMA (e.g., via serum concentrations of ravulizumab over time, serum free C5 concentrations over time, and serum total C5 concentrations over time).

Safety objectives include characterizing the safety profile of ravulizumab in adult and adolescent participants with HSCT-TMA (e.g., via incidence of treatment-emergent AEs and treatment-emergent SAEs, changes from baseline in vital signs and laboratory parameters, and incidence of ADAs and assessment of immunogenicity).

### 2. Overall Study Design

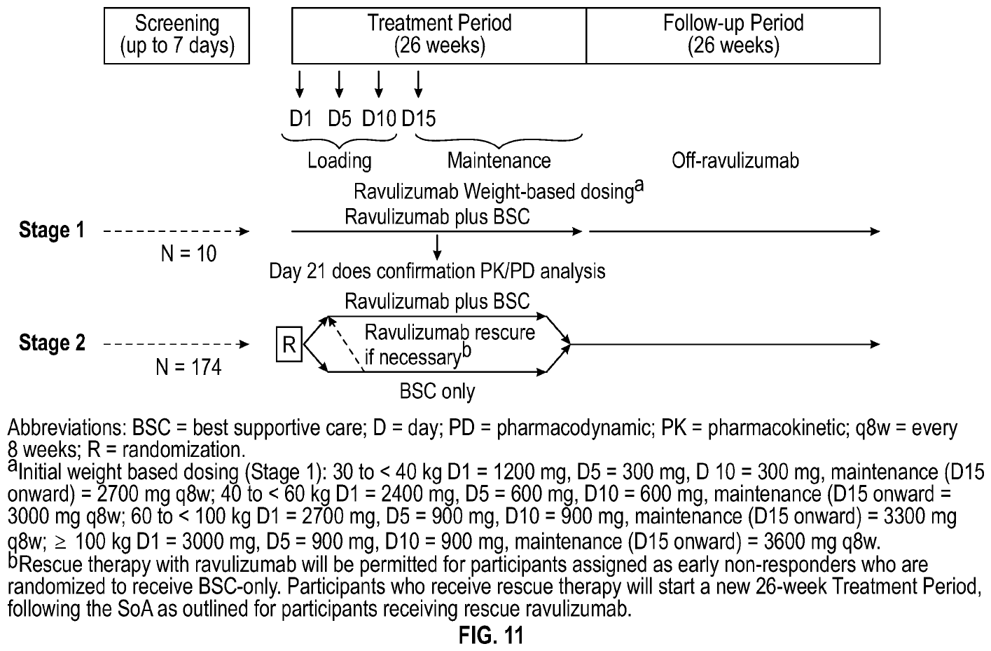

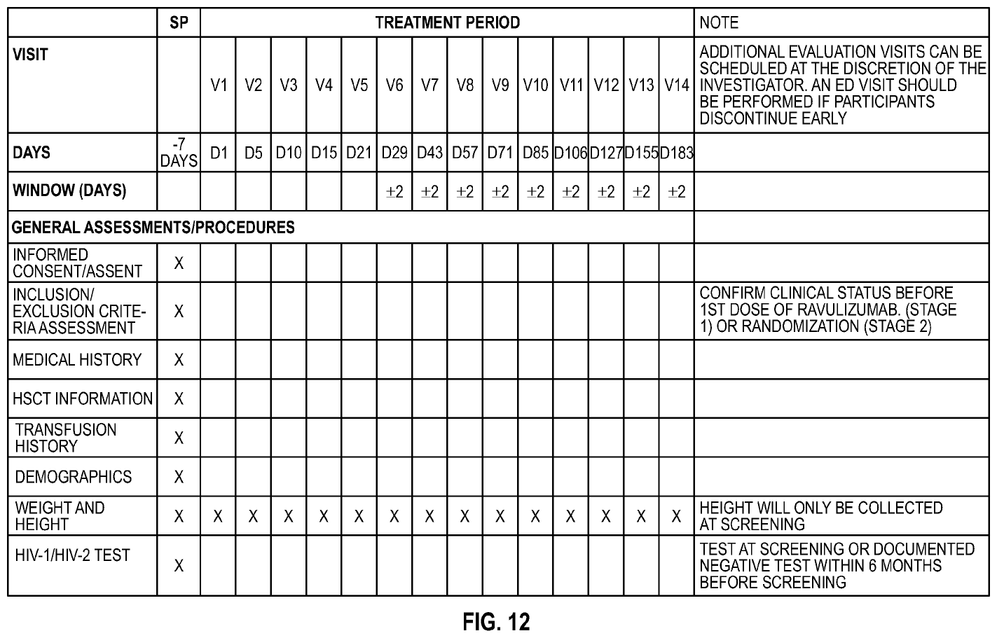

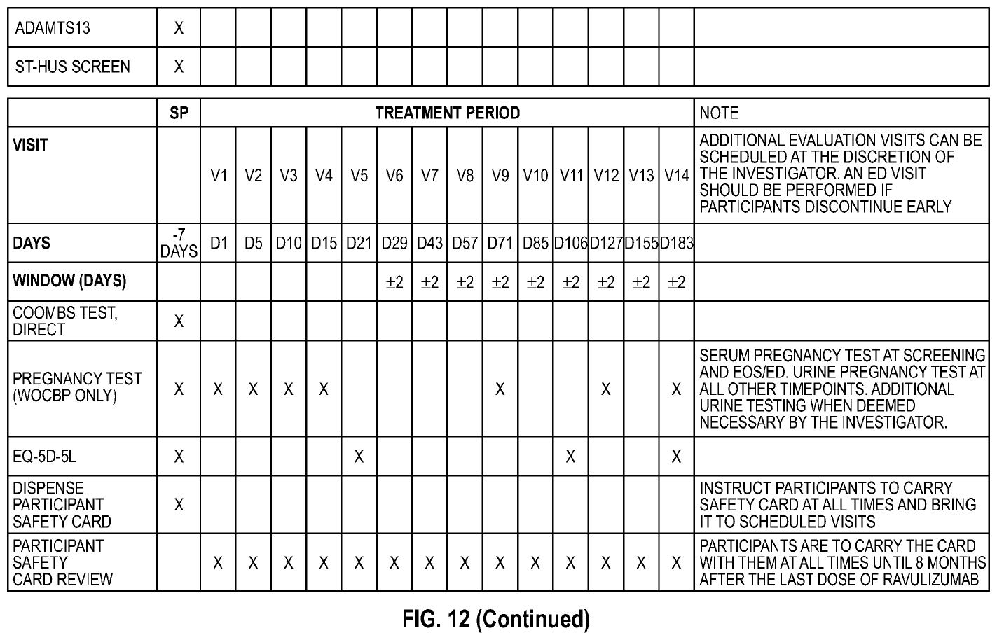

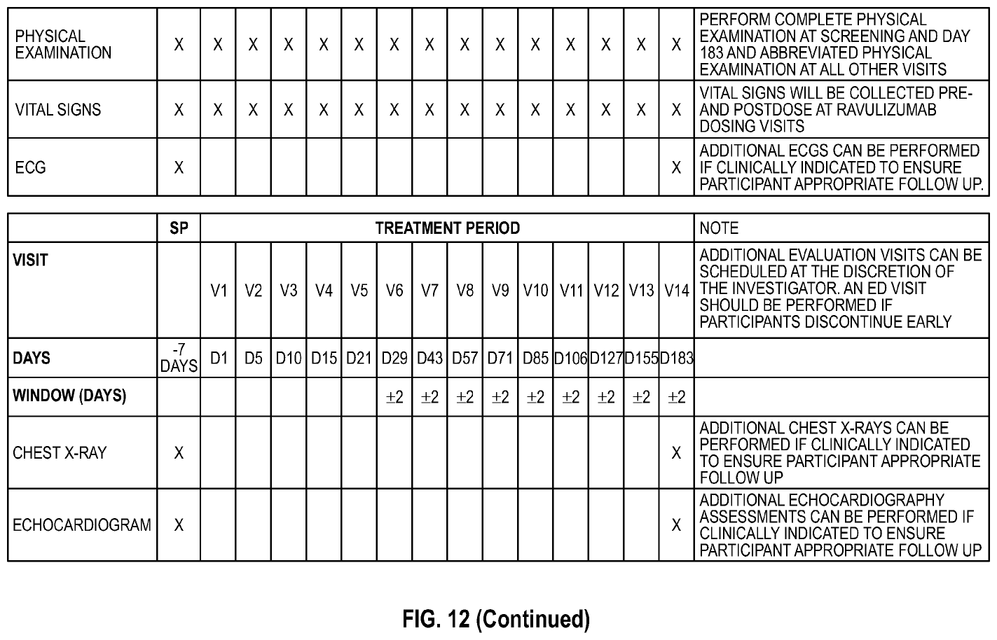

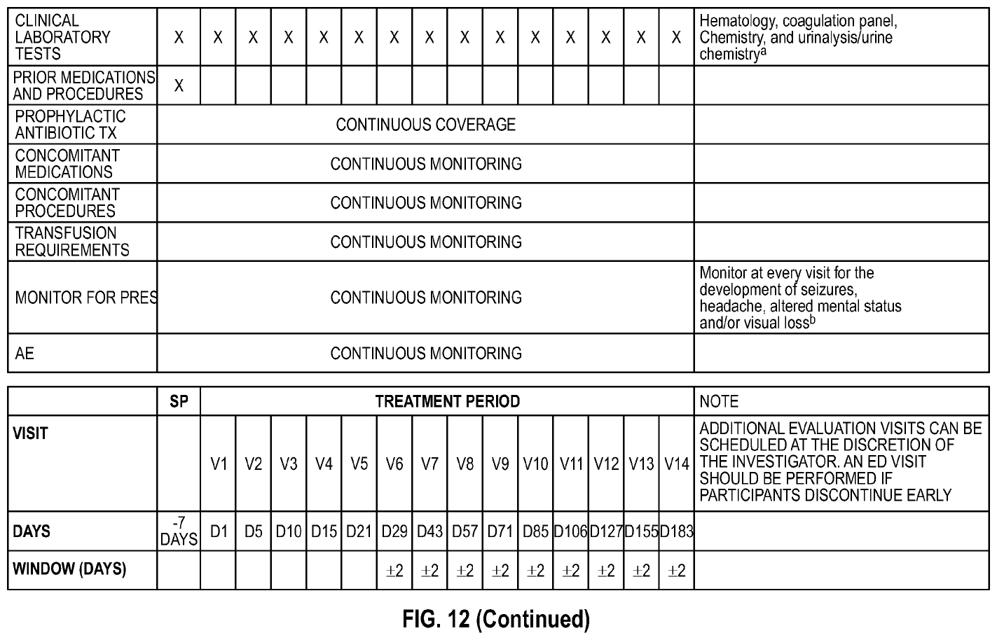

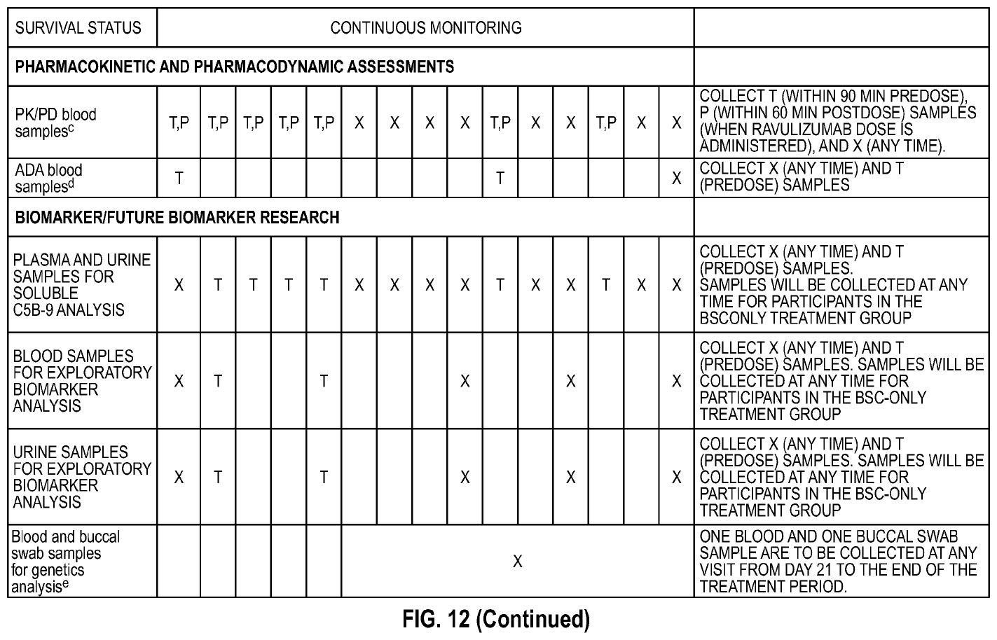

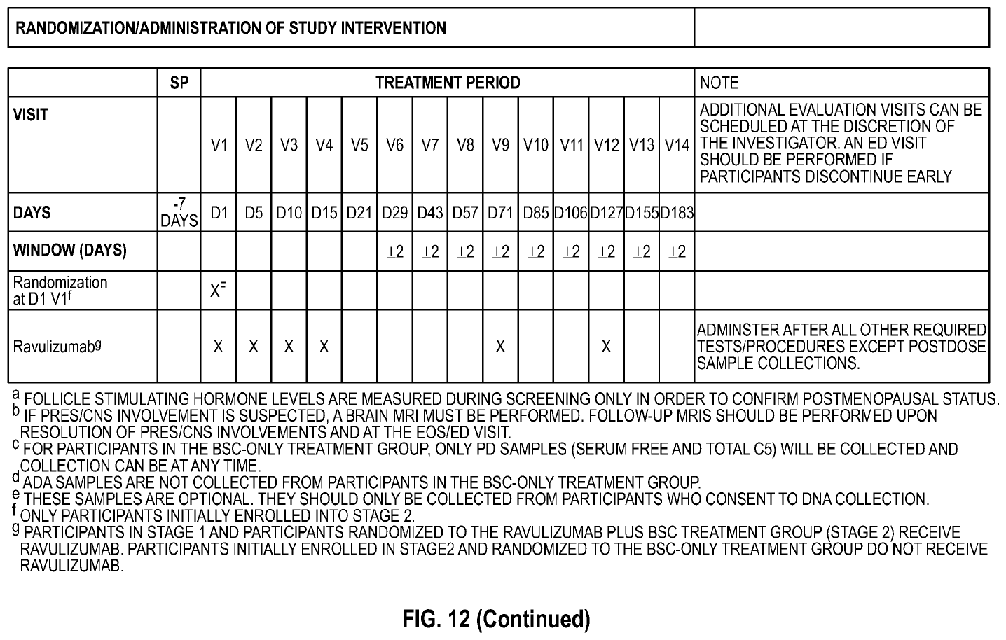

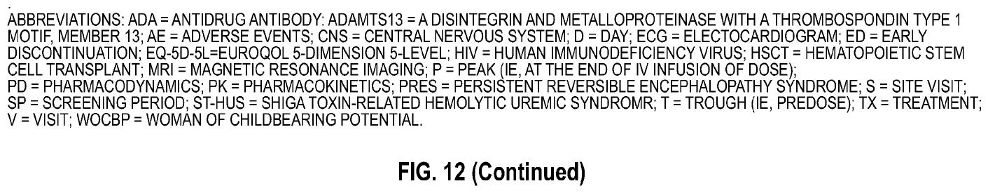

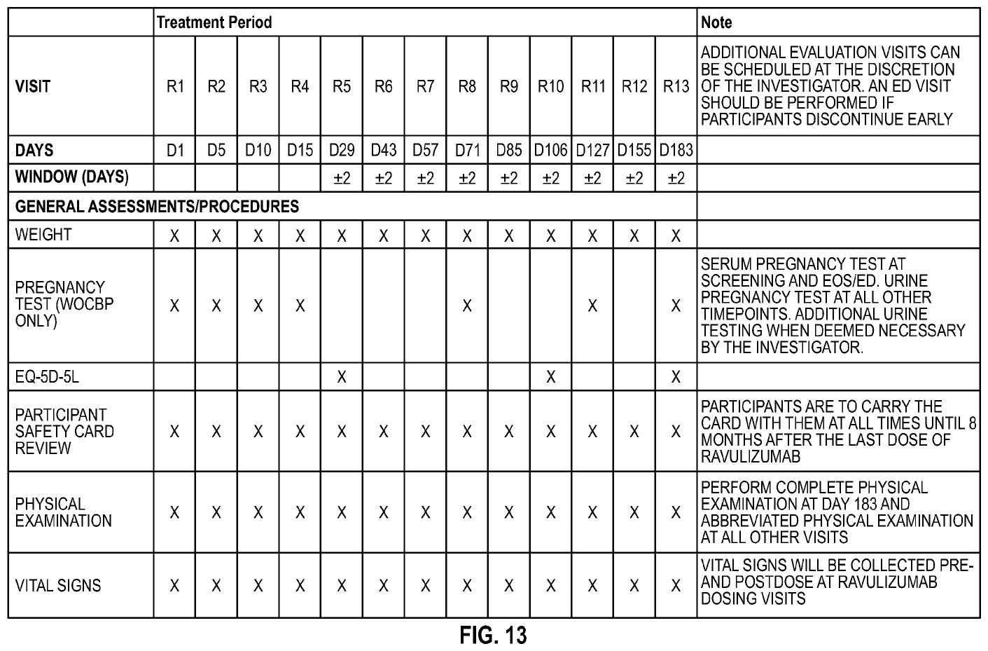

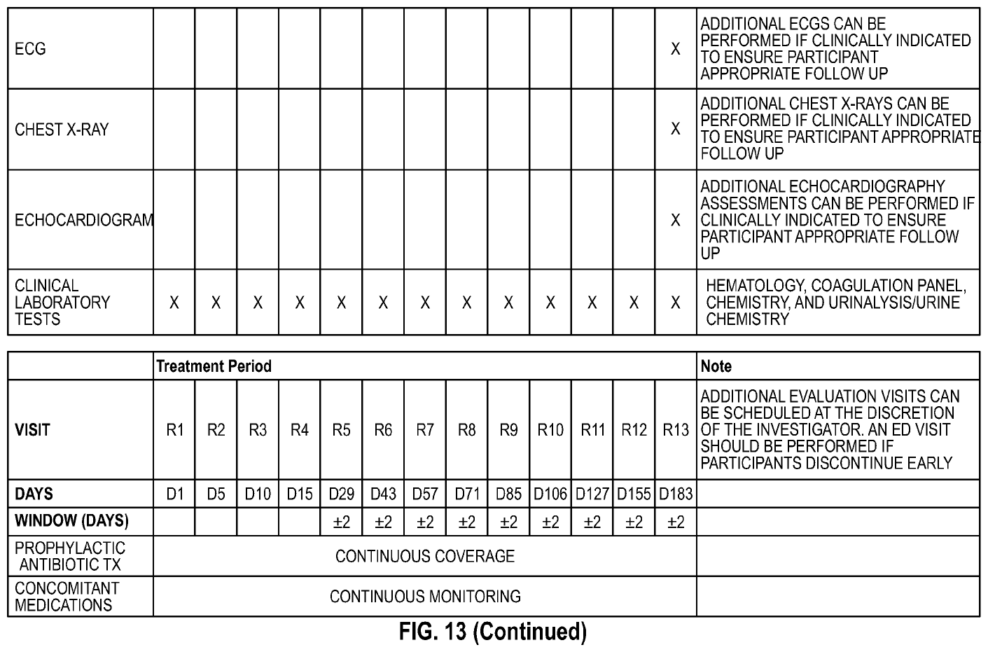

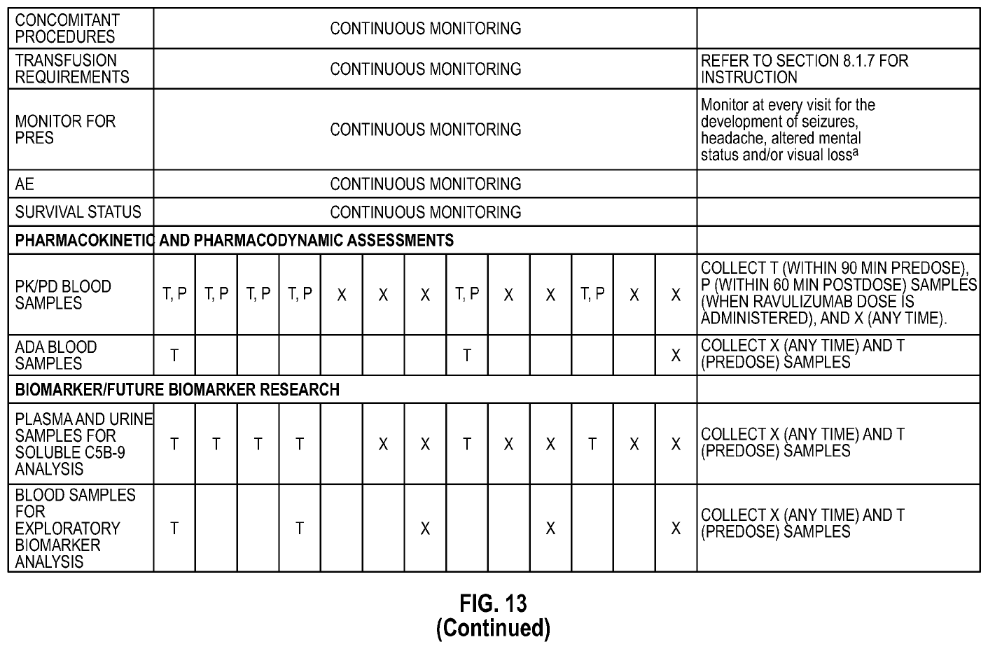

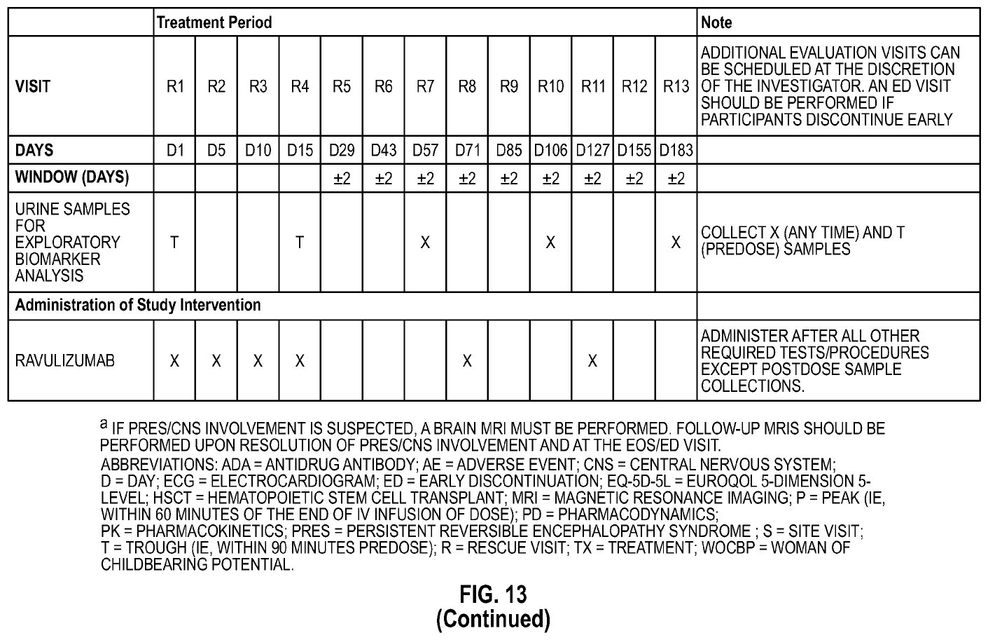

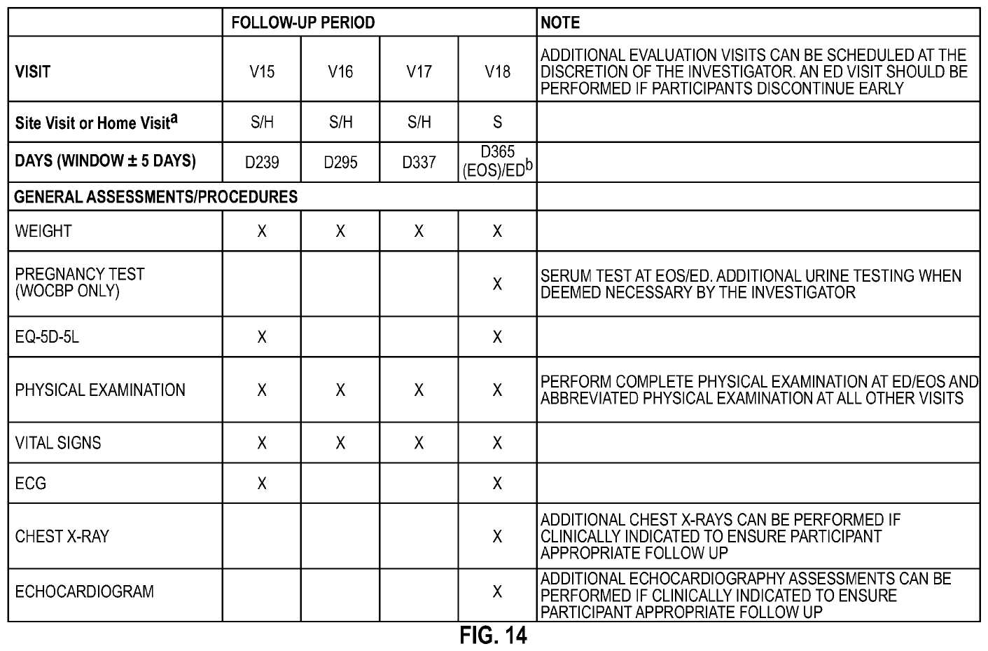

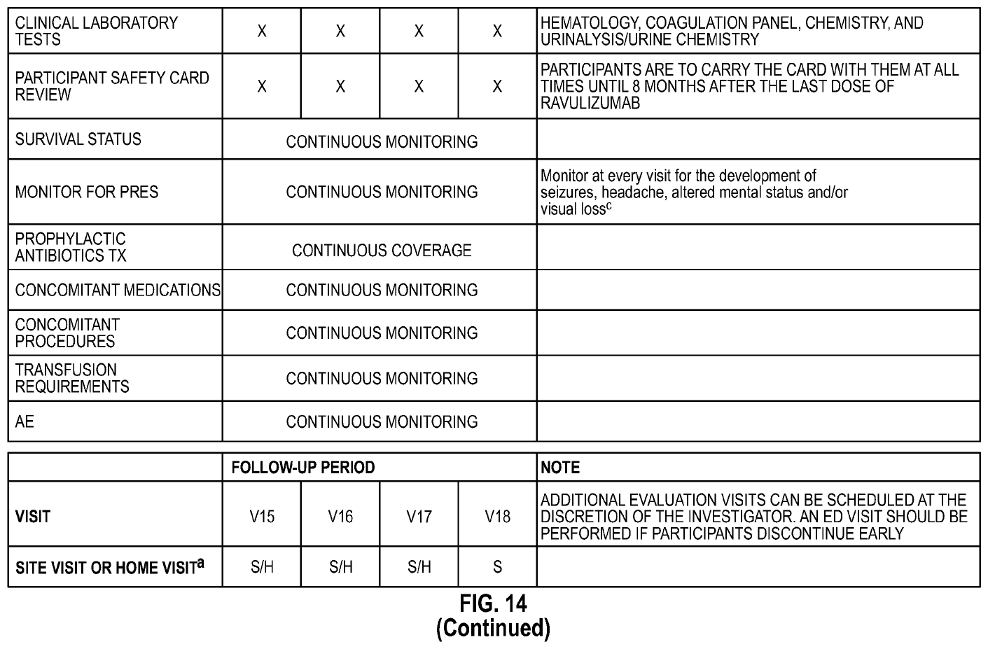

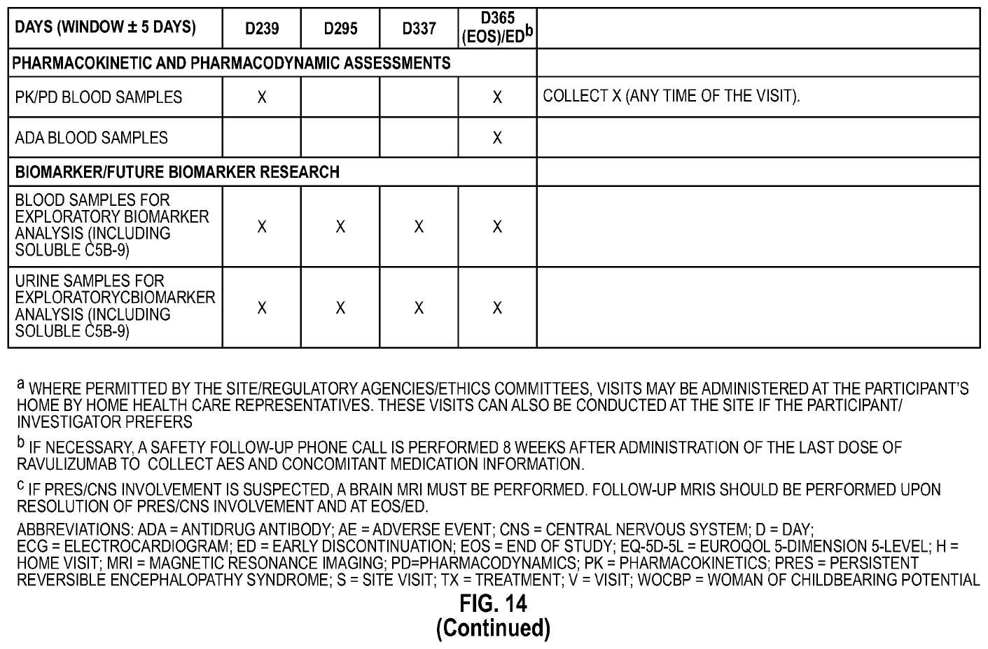

This is a two-stage open-label, randomized, multicenter study of ravulizumab in adolescent (12 to 17 years of age) and adult participants with HSCT-TMA. An overview of the study is set forth in FIG. 11. The schedule of activities for the screening and treatment Period (Stage 1 and Stage 2 Participants) is presented in FIG. 12. The schedule of activities for the treatment period (participants who receive ravulizumab rescue therapy) is presented in FIG. 13. The schedule of activities for the follow-up period is presented in FIG. 14.

In Stage 1, the dosing regimen for patients with HSCT-TMA is confirmed. In Stage 2, participants are randomized to receive either ravulizumab plus best supportive care (BSC) or BSC-only. Eligible participants include adults and adolescents who received HSCT within 6 months prior to Screening and subsequently developed TMA that does not resolve within 72 hours after withdrawal or adjustment of any TMA-associated medication and/or treatment of any associated underlying triggering condition.

Approximately 184 participants are enrolled to the study. This includes 10 participants in Stage 1 and 174 participants in Stage 2 (including at least 5 Japanese participants).

The study is conducted in two stages: a Dose Confirmation Stage (Stage 1) in 10 participants, followed by a Randomized Treatment Stage (Stage 2) once an adequate dosing regimen has been confirmed in Stage 1. For all participants enrolled (Stage 1 and Stage 2 participants), the study consists of 3 periods: a Screening Period of up to 7 days; a 26-week Treatment Period; and a 26-week Follow-up Period. All participants receive BSC for the duration of the study or as long as deemed necessary by the Investigator.

During Stage 1, all participants receive ravulizumab plus BSC. During Stage 2, participants are randomized 1:1 to receive either ravulizumab plus BSC or BSC-only. For each participant, the expected duration of study participation is up to 53 weeks. This includes the Screening Period (of up to 1 week), the Treatment Period (26 weeks), and the Follow up Period (26 weeks). If necessary, a safety follow up phone call is performed 8 weeks after the last dose of ravulizumab. The study duration is expected to be longer for participants who receive ravulizumab rescue therapy since they will receive ravulizumab for 26 weeks followed by a 26-week Follow-up Period after the decision has been made to administer rescue therapy with ravulizumab.

The 10 participants enrolled in Stage 1 receive the following weight-based ravulizumab dosing regimen via intravenous (IV) infusion:

TABLE 14
Ravulizumab Dosing

| | | Loading Phase Doses | | | Maintenance Doses |
|---|---|---|---|---|---|
| Weight^{a} | | Day 1 | Day 5 | Day 10 | Starting Day 15 |
| 30 to < 40 | kg | 1200 mg | 300 mg | 300 mg | 2700 mg q8w |
| 40 to < 60 | kg | 2400 mg | 600 mg | 600 mg | 3000 mg q8w |
| 60 to < 100 | kg | 2700 mg | 900 mg | 900 mg | 3300 mg q8w |
| ≥100 | kg | 3000 mg | 900 mg | 900 mg | 3600 mg q8w |

^{a}Weight-based dosing is based on the participant's body weight recorded at the day of the infusion visit. If the weight at the day of the infusion cannot be obtained, the weight recorded during the previous study visit may be used.
Abbreviation: q8w = every 8 weeks.

For participants in Stage 1, the PK and free complement component 5 (C5) data collected over the first 21 days is analyzed to confirm the adequacy of the initial dosing regimen to achieve complete inhibition of C5. This analysis may result in a modification to the dosing regimen, which is used in Stage 2. If necessary, additional participants may be enrolled into Stage 1 either prior to the analysis (e.g., if distribution of the initial 10 is not optimal) or after the analysis if the dosing regimen cannot be confirmed.

Following the dose confirmation assessment in Stage 1, approximately 174 participants are enrolled in Stage 2. In Stage 2, participants are randomized on Day 1 in a 1:1 ratio to receive either ravulizumab plus BSC or BSC-only. Permitted BSC measures include, but are not limited to, transfusion support, hemodynamic support, renal replacement therapy, and continued treatment of any associated comorbidity. Treatment with other complement inhibitors is not allowed during this study. Participants randomized to the ravulizumab plus BSC treatment group are treated with IV ravulizumab for 26 weeks. The ravulizumab dosing regimen for Stage 2 is either be the initial dosing regimen (above) or a modified dosing regimen (based on analyses performed in Stage 1).

Randomization is stratified at Stage 2 baseline based on geographical region (East Asia and Rest of the World), and graft versus host disease (GVHD) status at Screening (no GVHD, Grade I-II GVHD, or Grade III-IV GVHD). The criteria outlined by EBMT, National Institutes of Health (NIH), and the Center for International Blood and Marrow Transplant Research (CIBMTR) (Schoemans H M, et al., Bone Marrow Transplant. 2018; 53(11):1401-1415) is set forth in Table 15 and used for assessing GVHD grade in this study.

TABLE 15
GVHD Grading

Grade

| | |
|---|---|
| I | Rash on ≤50% of skin, no liver or gut involvement |
| II | Rash on >50% of skin, bilirubin 2-3 mg/dL, or diarrhea |
| | 500-1000 mL/day, or persistent nausea |
| III | Bilirubin 3-15 mg/dL, or gut stage 2-4, diarrhea >1000 |
| | mL/day, or severe abdominal pain with or without ileus |
| IV | Generalized erythroderma with bullous formation, or |
| | bilirubin >15 mg/dL |
| Not | Acute GVHD present but cannot be graded |
| applicable | |

After completion of the 26-week Treatment Period, all participants enter the Follow-up Period and remain in the study for 26 weeks without further ravulizumab administration. In the case of clinical need for extended treatment (e.g., if a participant starts to exhibit TMA response late in the Treatment Period), the Investigator and the Medical Monitor can mutually agree on additional dosing into the Follow-up Period based on the weight-based dosing regimen.

For participants who receive ravulizumab and withdraw from the study or participants who are administered ravulizumab during the Follow-up Period, a safety follow-up phone call is performed 8 weeks after the last dose of ravulizumab if that date is later than the date of the End of Study (EoS) Visit.

If a ravulizumab-treated participant in either stage meets criteria for TMA relapse or loss of TMA response during the Follow-up Period, the participant can receive ravulizumab retreatment

An evaluation for early assignment of non-response (based on clinical worsening or no improvement) is performed for both treatment groups, and early non-response is assigned if the participant meets the defined criteria for clinical worsening or no improvement after 21 days of treatment initiation. Ravulizumab rescue therapy is permitted for participants assigned as early non-responders who are randomized to the BSC-only treatment group. These participants enter a 26-week ravulizumab Treatment Period followed by a 26-week Follow-up Period after the decision has been made to administer rescue therapy with ravulizumab.

### 3. Study Population

Approximately 184 participants are enrolled to the study. This includes 10 participants in Stage 1 and 174 participants in Stage 2 (including at least 5 Japanese participants).

Participants are eligible for enrollment in the study only if they satisfy all of the following criteria:
1. Participant must be ≥12 years of age at the time of signing the informed consent form (ICF) or assent form;
2. Adult and adolescent participants who received HSCT within the past 6 months at the time of Screening;
3. A TMA diagnosis, based on all of the following criteria occurring simultaneously: (a) de novo thrombocytopenia or platelet transfusion refractoriness, where: de novo thrombocytopenia is defined as new decline in platelet count to ≤50,000/mm³ and transfusion refractoriness is defined as failure of the platelet increment 10 60 minutes post transfusion to exceed the transfusion trigger, or a rise of less than 10×10⁹/L 20-24 hours post-transfusion after at least 2 platelet transfusions, (b) de novo anemia (defined as a new decline in hemoglobin to ≤8 g/dL) or increase in transfusion requirements (defined as the need to administer more frequent transfusions in order to maintain hemoglobin ≥8 g/dL), (c) either one of the following markers of hemolysis: LDH>1.5×ULN or presence of schistocytes ≥2 high power field (HPF) in peripheral blood smear, (d) proteinuria on spot urinalysis where proteinuria is defined as protein/creatinine ratio ≥0.5 mg/mg. The presence of proteinuria should be confirmed by a second measurement (with the 2 measurements at least 4 hours apart); (e) presence of hypertension defined as the presence of any one of the 3 conditions: a systolic pressure ≥139 mm Hg or a diastolic pressure greater than 80 mm Hg on 2 consecutive measurements at least 1 hour apart; requirement for new antihypertensive medication after HSCT (for a participant not on antihypertensive medication prior to HSCT), or for participants with underlying hypertensive disease, a change in their antihypertensive regimen or the addition of new antihypertensive agents required to treat hypertension;
4. Participants must have HSCT-TMA that persists despite initial management of any triggering condition (persists for at least 72 hours after management of triggering agent/condition): withdrawal or dose reduction of the offending agent (e.g., CNIs), treatment of any underlying infection, or treatment of underlying GVHD);
5. Body weight ≥30 kg at Screening;
6. Male or female. Contraceptive use by men or women is consistent with local regulations regarding the methods of contraception for those participating in clinical studies Male participants agree to use contraception during the Treatment Period and for at least 8 months after the final dose of ravulizumab and refrain from donating sperm during this period A female participant is eligible to participate if she is not pregnant, not breastfeeding, and meets at least one of the following conditions: not a woman of childbearing potential (WOCBP) or is a WOCBP and using a highly effective and acceptable contraceptive method during the Treatment Period and for at least 8 months after the final dose of ravulizumab A WOCBP must have a negative serum pregnancy test at Screening and a negative urine pregnancy test before the first dose of ravulizumab;
7. Participants must be vaccinated against meningococcal infections if clinically feasible, according to institutional guidelines for immune reconstitution after HSCT. Participants <18 years of age must be re-vaccinated against Haemophilus influenzae type b (Hib) and Streptococcus pneumoniae if clinically feasible, according to institutional guidelines for immune reconstitution after HSCT. All participants are administered coverage with prophylactic antibiotics according to institutional posttransplant infection prophylaxis guidances including coverage against N. meningiditis for at least 2 weeks after meningococcal vaccination. Participants who cannot receive meningococcal vaccine should receive antibiotic prophylaxis coverage against N. meningiditis the entire Treatment Period and for 8 months following the final dose of ravulizumab; and
8. Capable of giving signed informed consent or assent. Participants are excluded from the study if any of the following criteria apply:
1. Known familial or acquired ‘a disintegrin and metalloproteinase with a thrombospondin type 1 motif, member 13’ (ADAMTS13) deficiency (activity <5%);
2. Known Shiga toxin-related hemolytic uremic syndrome (ST-HUS) as demonstrated by a positive test for Shiga toxin or culture of Shiga toxin producing bacteria;
3. Positive direct Coombs test;
4. Diagnosis or suspicion of disseminated intravascular coagulation (DIC) according to the International Society of Thrombosis and Haemostasis (ISTH) scoring criteria as set forth in Taylor et al., Thromb Haemost. 2001; 86(5):1327-1330 and Tables 2-3;
5. Known bone marrow/graft failure;
6. Diagnosis of veno-occlusive disease (VOD), regardless of severity, according to the European Society for Blood and Bone Marrow Transplantation (EBMT) criteria as set forth by Mohty M, et al., Bone Marrow Transplant. 2016; 51(7):906-912 and in Table 16;
7. Human immunodeficiency virus (HIV) infection (evidenced by HIV-1 or HIV-2 antibody titer, or documented negative HIV-1/HIV-2 tests within 6 months prior to Screening);
8. Unresolved meningococcal disease;
9. Presence or suspicion of sepsis (treated or untreated) within 7 days prior to Screening;
10. Pregnancy or breastfeeding;
11. Hypersensitivity to murine proteins or to 1 of the excipients of ravulizumab;
12. Any medical or psychological condition that, in the opinion of the Investigator or Sponsor, could increase the risk to the participant by participating in the study or confound the outcome of the study;
13. Previously or currently treated with a complement inhibitor;
14. Participation in another interventional treatment study or use of any experimental therapy (except for the use of currently approved medications being investigated alone or in combination for: treatment of the underlying disease; conditioning regimen: GVHD prophylaxis treatment; infections prophylaxis; or posttransplant infection treatment) within 30 days before initiation of ravulizumab on Day 1 in this study or within 5 half lives of that investigational product, whichever is greater

TABLE 16
EBMT Criteria for VOD in Adults

| | | | | Very Severe |
|---|---|---|---|---|
| | Mild^{a} | Moderate^{a} | Severe | (MOD/MOF^{b}) |
| Time since first | >7 days | 5-7 days | ≤4 days | Any time |
| clinical symptoms | | | | |
| of SOS/VOD^{c} | | | | |
| Bilirubin (mg/dL) | ≥2 and <3 | ≥3 and <5 | ≥5 and <8 | ≥8 |
| Bilirubin (μmol/L) | ≥34 and <51 | ≥51 and <85 | ≥85 and <136 | ≥136 |
| Bilirubin kinetics | Doubling within 48 hours | | | |
| Transaminases | ≤2 × normal | >2 and ≤5 × | >5 and ≤8 × | >8 × normal |
| | | normal | normal | |
| Weight increase | <5% | ≥5% and <10% | ≥5% and <10% | ≥10% |
| Renal function | <1.2 × BAT | ≥1.2 and <1.5 × | ≥1.5 and <2 × | ≥2 × BAT or |
| | | BAT | BAT | other signs of |
| | | | | MOD/MOF |

Abbreviations: BAT = baseline at transplant; MOD = multi-organ dysfunction; MOF = multi-organ failure; SOS = sinusoidal obstruction syndrome; VOD = veno-occlusive disease.
Participants belong to the category that fulfills ≥2 criteria. If participants fulfill ≥2 criteria in 2 different categories, they must be classified in the most severe category. Participants weight increase ≥5% and <10% is considered by default as a criterion for severe SOS/VOD; however, if participants do not fulfill other criteria for severe SOS/VOD, weight increase ≥5% and <10% is therefore considered as a criterion for moderate SOS/VOD.
^{a}In the case of presence of ≥2 risk factors for SAS/VOD, participants should be in the upper grade.
^{b}Participants with multi-organ dysfunction must be classified as very severe.

### 4. Study Intervention

Study intervention is defined as any investigational intervention(s), marketed product(s), placebo, or medical device(s) intended to be administered to a study participant according to the study protocol. Details regarding ravulizumab are set forth in Table 5.

The study is conducted in an open-label manner. To minimize bias, participants are randomized on Day 1 after the Investigator has verified that they are eligible. Randomization is stratified by region (East Asia and Rest of the World), as well as GVHD status at Screening (no GVHD; Grade I-II GVHD; and Grade III-IV GVHD where grade is assigned using the International Bone Marrow Transplant Registry Severity Index for grading acute GVHD set forth in Table 15). Participants are randomized 1:1 either to the ravulizumab plus BSC treatment group or to the BSC-only treatment group. Randomization is performed centrally using Interactive Response Technology (IRT).

To prevent the possibility of rescue-caused bias in assessing the primary endpoint, an evaluation for early assignment of non-response based on clinical worsening or no improvement is performed for both treatment groups.

During this study, participants receive all ravulizumab dosing directly from the Investigator or designee, under medical supervision at the study site. The date and time of each dose administered in the clinic is recorded in the source documents and in the case report forms (CRFs). The dose of study intervention and study participant identification is confirmed at the time of dosing by a member of the study site staff other than the person administering the study intervention.

In this study, all participants will receive BSC as background therapy. BSC is determined by the Investigator according to institutional practices and participant characteristics. BSC measures include but are not limited to: (a) transfusion support: transfusion support is provided as required per institutional guidelines and based on the participant's clinical condition. In general, transfusion support is recommended as follows per the Joint United Kingdom (UK) Blood Transfusion and Tissue Transplantation Services Professional Advisory Committee (JAPC): red blood cell transfusion should be considered in participants with hemoglobin ≤7 g/dL or in participants with symptomatic anemia (e.g., dyspnea, and/or tachycardia) with hemoglobin ≤8 g/dL, prophylactic platelet transfusions should be given to participants as required with a transfusion trigger of 10×10⁹/L, and an increase in the platelet transfusion threshold to 20×10⁹/L is justified in participants who are febrile and/or receiving antibiotic therapy for suspected bacterial or fungal infection. Participants who receive transfusions of RBCs or platelets require supplemental doses of ravulizumab, (b) corticosteroids, (c) dialysis, and antihypertensive medications. Supportive care measurements are recorded in the appropriate CRF.

Supportive care for underlying conditions (e.g., HSCT, GVHD) is permitted during the course of the study. Use of currently approved medications being investigated alone or in combination for the treatment of the underlying disease, conditioning regimen, GVHD prevention regimen, GVHD treatment, infection prophylaxis, or infection treatment. Other concomitant medication may be considered on a case-by-case basis by the Investigator

The following are not allowed: (a) eculizumab or other agents that act on the complement pathway, (b) use of PE is prohibited during the study (c) use of rituximab, (d) IV immunoglobulin (Ig) (unless for an unrelated medical need, e.g., hypergammaglobulinemia) (e) defibrotide (except for participants with confirmed VOD that develops during the study), and (f) use of any experimental interventions or therapies.

Rescue with ravulizumab is permitted for participants in the BSC-only treatment group. The date and time of rescue medication administration must be recorded.

The adequacy of the proposed dosing regimen is confirmed through initial analysis of PK/PD in the first 10 participants enrolled in Stage 1. If the proposed dosing regimen does not result in the anticipated degree of free C5 inhibition, the dosing regimen is modified (increased) for subsequent participants according to PK/PD data.

Ravulizumab is not administered to participants after the end of the study. Upon completion of the last study visit, participants return to the care of their treating physician.

### 5. Discontinuation of Study Intervention

In rare instances, it may be necessary for a participant to permanently discontinue (definitive discontinuation) the study intervention. Participants should be considered for discontinuation from intervention if any of the following occur during the study: (1) serious hypersensitivity reaction, (2) use of disallowed medication, (3) pregnancy or planned pregnancy, (4) it is deemed necessary for the participant.

If the study intervention is definitively discontinued, every effort should be made to have the participant continue the study visits as per the Schedule of Activities for safety follow-up. If the participant does not agree to continue with the Schedule of Activities visits, the following activities should be completed: (1) early discontinuation (ED) assessments to be performed are indicated in the Schedule of Activities, (2) the site should follow the participant for at least 8 weeks after the final dose of ravulizumab via a safety phone call to collect any AEs and/or concomitant medications, (3) information about survival status at 6 months and 1 year after first dose of ravulizumab (for participants in Stage 1) or post-randomization (for participants in Stage 2) should be collected. If a participant's survival status is not available at the time of discontinuation, sites should attempt to collect the status (e.g., via public records, or telephone call).

All efforts should be made to ensure participants are willing to comply with study participation prior to conducting the Screening procedures. The study staff should notify Alexion and their site monitor of all study withdrawals as soon as possible. The reason for participant discontinuation must be recorded in the source documents and CRF.

A participant may withdraw from the study at any time at his/her own request or may be withdrawn at any time at the discretion of the Investigator for safety, behavioral, compliance, or administrative reasons.

At the time of discontinuing from the study, if possible, an ED Visit should be conducted, as shown in the Schedule of Activities. If the participant does not agree to an ED Visit, the site follows the participant for at least 8 weeks after the final dose of ravulizumab via a safety phone call to collect any AEs and/or concomitant medications. In addition, information about survival status at 6 months and 1 year after first dose of ravulizumab (for participants in Stage 1) or post-randomization (for participants in Stage 2) is collected. If a participant's survival status is not available at the time of discontinuation, sites attempt to collect the status (e.g., via public records, or telephone call). The participant is permanently discontinued both from the study intervention and from the study at that time. 7.3.

A participant is considered lost to follow-up if he or she repeatedly fails to return for scheduled visits and is unable to be contacted by the study site.

### 6. Study Assessments and Procedures

In Stage 1, ravulizumab is administered by IV infusion according to the dosing regimen in Table 17. The dose confirmation analysis is triggered when the tenth participant completes Visit 5 on Day 21 and includes all PK/PD data collected by that time. This analysis confirms the adequacy of the initial dosing regimen and supplemental dosing regimen to achieve and maintain complete suppression of C5 throughout the dosing interval. This analysis may result in an adjusted dosing regimen for participants in Stage 1 and the participants enrolled in Stage 2. Participants in Stage 1 receive the adjusted dosing regimen for the remainder of the Treatment Period.

TABLE 17
Initial Dosing Regimen

| | | Loading Phase Doses | | | Maintenance Doses |
|---|---|---|---|---|---|
| Weight^{a} | | Day 1 | Day 5 | Day 10 | Starting Day 15 |
| 30 to <40 | kg | 1200 mg | 300 mg | 300 mg | 2700 mg q8w |
| 40 to <60 | kg | 2400 mg | 600 mg | 600 mg | 3000 mg q8w |
| 60 to <100 | kg | 2700 mg | 900 mg | 900 mg | 3300 mg q8w |
| ≥100 | kg | 3000 mg | 900 mg | 900 mg | 3600 mg q8w |

^{a}Weight-based dosing is based on the participant's body weight recorded at the day of the infusion visit. If the weight at the day of the infusion cannot be obtained, the weight recorded during the previous study visit may be used.
Abbreviation: q8w = every 8 weeks.

In the case of clinical need for extended treatment (e.g., if a participant starts to exhibit TMA response late in the Treatment Period), the Investigator and the Medical Monitor can mutually agree on additional dosing into the Follow-up Period based on the weight-based dosing regimen in Table 17. Unscheduled visits can be arranged as needed for ravulizumab administration. The following assessments must be performed at any unscheduled visit in which ravulizumab is administered: (a) a urine pregnancy test (WOCBP only), abbreviated physical examination, PK/PD blood sample collection, vital signs, clinical laboratory tests, C5b-9 blood sample collection, and C5b-9 urine sample collection.

Supplemental doses of ravulizumab are allowed according to the criteria outlined in Table 18.

TABLE 18
Supplemental Dosing of Ravulizumab

| Qualified reason | Supplemental dose of ravulizumab |
|---|---|
| Participants who demonstrate clinical worsening | For the <2 weeks from the most recent |
| defined as 2 or more of the following: doubling | maintenance dose |
| of serum creatinine from baseline, increase in | Dose are determined following assessment |
| LDH >25% compared to baseline, reduction in | For ≥2 weeks after the most recent |
| platelets of >25% compared to baseline, | maintenance dose |
| reduction in hemoglobin >10% compared to | A bodyweight-based maintenance dose can |
| baseline, increased requirement for platelets or | be given |
| RBCs at any time | |
| Participants with no improvement after 21 days | |
| treatment initiation, determined by an increase in | |
| platelet count of <10% relative to baseline | |
| Participants who receive RBC and/or platelet | For participants with body weight <40 |
| transfusions | kg, 300 mg is administered the day |
| | after ≥1 RBC or platelet unit |
| | transfusions. Supplemental doses are |
| | not to be administered on regularly |
| | scheduled dosing days (Days 1, 5, 10, |
| | 15, 71, or 127). |
| | For participants with body weight ≥40 |
| | kg, 600 mg is administered the day |
| | after ≥1 RBC or platelet unit |
| | transfusions. Supplemental doses are |
| | not to be administered on regularly |
| | scheduled dosing days (Days 1, 5, 10, |
| | 15, 71, or 127). |
| | These supplemental doses are confirmed at |
| | the time of Stage 1 dose confirmation |
| | analysis and adjusted before Stage 2 if |
| | necessary. |

Unscheduled visits can be arranged as needed for supplemental ravulizumab administration. The following assessments must be performed during any unscheduled visit in which ravulizumab is administered: urine pregnancy test (WOCBP only), abbreviated physical examination, PK/PD blood sample collection, vital signs, clinical laboratory tests, C5b-9 blood sample collection, and C5b-9 urine sample collection.

It is expected that participants who entered into Stage 1 are treated for the 26-week Treatment Period and then be off-treatment for the 26-week Follow-up Period. A participant can receive ravulizumab during the Follow-up Period if: he/she meets criteria for TMA relapse or loss of TMA response and the Investigator believes that the participant potentially benefits from retreatment, initiates a discussion with the Medical Monitor, and provides a medically justified rationale.

If retreatment is determined necessary, the participant continues to follow the Schedule of Activities as planned. The participant receives the ravulizumab weight-based dose according to the dosing regimen described in Table 17.

Unscheduled visits can be arranged as needed for ravulizumab administration. The following assessments must be performed during any unscheduled visit in which ravulizumab is administered: urine pregnancy test (WOCBP only), abbreviated physical examination, PK/PD blood sample collection, vital signs, clinical laboratory tests, C5b-9 blood sample collection, and C5b-9 urine sample collection.

Participants in Stage 2 randomized to ravulizumab plus BSC receive the confirmed adequate dosing regimen resulting from Stage 1 PK/PD analysis. In the case of clinical need for extended treatment (e.g., if a participant starts to exhibit TMA response late in the Treatment Period), the Investigator and the Medical Monitor may mutually agree on additional dosing into the Follow-up Period based on the weight-based dosing regimen in Table 17. Unscheduled visits can be arranged as needed for ravulizumab administration. The following assessments must be performed at any unscheduled visit in which ravulizumab is administered: urine pregnancy test (WOCBP only), abbreviated physical examination, PK/PD blood sample collection, vital signs, clinical laboratory tests, C5b-9 blood sample collection, and C5b-9 urine sample collection.

An evaluation for early assignment of non-response (based on clinical worsening or no improvement) is performed for both treatment groups, and rescue therapy with ravulizumab is permitted for participants assigned as early non-responders who are randomized to the BSC-only treatment group.

Early non-response are assigned if the participant meets either of the following criteria: (1) Defined clinical worsening i.e., presence of 2 or more of the following: increase in LDH>25% compared to baseline, reduction in platelets of ≥25% compared to baseline, reduction in hemoglobin >10% compared to baseline, progressive increase in requirement for platelet transfusions to maintain a platelet count ≥20,000/mm³, progressive increase in requirements for RBC transfusions to sustain hemoglobin ≥8 g/dL, doubling of serum creatinine value from baseline OR (2) no improvement after Day 21, determined by an increase in platelet count of <10% relative to baseline. Participants who are assigned as early non-responders and who then receive rescue ravulizumab receive the ravulizumab dosing regimen confirmed in Stage 1 and follow the Schedule of Activities as outlined for participants receiving rescue ravulizumab.

Supplemental doses of ravulizumab for ravulizumab-treated participants in Stage 2 is allowed. The criteria for supplemental dosing are similar to those for Stage 1.

Unscheduled visits can be arranged as needed for supplemental ravulizumab administration. The following assessments must be performed during any unscheduled visit in which ravulizumab is administered: urine pregnancy test (WOCBP only), abbreviated physical examination, PK/PD blood sample collection, vital signs, clinical laboratory tests, C5b-9 blood sample collection, and C5b-9 urine sample collection.

It is expected that participants who entered into Stage 2 are treated for the 26-week Treatment Period and then be off-treatment for the 26-week Follow-up Period. A participant can receive ravulizumab during the Follow-up Period if he/she meets criteria for TMA relapse or loss of TMA response. The retreatment dosing in Stage 2 is the same as for Stage 1.

If retreatment is determined to be necessary, the participant continues to follow the Schedule of Activities as planned. The participant receives the ravulizumab weight-based dose according to the dosing regimen described in Table 17.

Unscheduled visits mcanay be arranged as needed for ravulizumab administration. The following assessments must be performed during any unscheduled visit in which ravulizumab is administered: urine pregnancy test (WOCBP only), abbreviated physical examination, PK/PD blood sample collection, vital signs, clinical laboratory tests, C5b-9 blood sample collection, and C5b-9 urine sample collection.

The participant's relevant medical history, including prior and concomitant conditions/disorders (including TMA diagnosis), treatment history, and family history of relevant diseases are evaluated at Screening by the Investigator and documented in the source documents and CRF.

Information collected includes transplant modality, hematopoietic stem cell origin, transplant indication, history of prior HSCT, conditioning regimen, presence of GVHD, engraftment status, and any other transplant complication(s) or relevant information on the peritransplant period.

The number and volume of transfusions during the Screening Period is documented on the transfusion history CRF. The information collected includes date of the transfusion, number of units, and volume of each blood component given. Transfusions during the study must be recorded on the transfusions CRF. The information to be collected includes date of the transfusion, number of units, and volume of each blood component given.

Due to its mechanism of action, the use of ravulizumab increases the participant's susceptibility to infection due to N. meningitidis. To reduce the risk of infection, participants must be vaccinated against N. meningitidis if clinically feasible. Vaccines against serotypes A, C, Y, W135, and B, where available, are recommended to prevent common pathogenic meningococcal serotypes.

As HSCT results in immunosuppression due to ablative therapy, concomitant medication, or due to underlying disease, vaccine antibody titers will decrease 1-4 years after the transplant if the patient is not revaccinated. It is recommended that sites follow the relevant immune reconstitution guidelines or local practice guidelines for immune reconstitution for re-vaccination of participants who have undergone HSCT.

Participants are administered prophylactic antibiotics for meningococcal infection until at least 2 weeks after vaccination. Vaccination may not be sufficient to prevent meningococcal infection. Consideration should be given per official guidance and local practice on the appropriate use of prophylactic antibacterial agents. All participants should be monitored for early signs of meningococcal infection, evaluated immediately if infection is suspected, and treated with appropriate antibiotics, if necessary. Participants who cannot be vaccinated must receive antibiotic prophylaxis for the entire treatment period and for 8 months following the final dose of ravulizumab.

If clinically feasible, participants <18 years of age are also vaccinated against Hib and S. pneumoniae according to the appropriate immune reconstitution guidelines and participants who have undergone HSCT must be re-vaccinated. Participants who cannot be vaccinated receive antibiotic prophylaxis for the entire treatment period and for 8 months following the final dose of ravulizumab. Vaccination status and administration of any vaccines, including those for N. meningitidis, Hib, and S. pneumoniae is recorded on the CRF.

### 7. Efficacy Assessments

Laboratory assessments to determine TMA response are performed according to the Schedule of Activities and assessed against predetermined criteria for TMA response.

For time to TMA response, partial TMA response, loss of TMA response, and TMA relapse, the relevant clinical laboratory assessments are performed according to the Schedule of Activities and assessed against predetermined criteria for TMA response.

Clinical laboratory assessments are performed according to the Schedule of Activities and assessed against predetermined criteria for hematologic response.

Participants' survival status is collected throughout the study. Information on any events of deaths and cause of death occurring during the study is collected.

Change from baseline in organ dysfunction (end organ involvement, i.e., renal system, cardiovascular system, CNS, pulmonary system, GI system) is described for participants in each randomized treatment group and in participants who received rescue therapy with ravulizumab. The following parameters to assess organ dysfunction are assessed:

Renal dysfunction: Kidney status is assessed by measuring protein/creatinine ratio, serum creatinine, and calculating estimated glomerular filtration rate (eGFR). For participants who require dialysis, change from baseline in dialysis requirements is recorded.

Cardiopulmonary involvement: Chest X-rays, electrocardiogram (ECG), and echocardiography to assess the presence of signs of pulmonary or cardiovascular involvement (including, but not limited to, pulmonary hypertension, pleural effusion, and pulmonary edema) is performed. In addition, use of any ventilatory or respiratory support is reported in the participant's CRF, along with associated oximetry.

Hypertension is assessed by blood pressure measurement at every visit as part of vital signs collection, and analysis of concomitant medications to control the hypertension.

Central nervous system involvement: Participants are assessed at every visit for signs of persistent reversible encephalopathy syndrome (PRES), including headache, confusion, visual loss, and seizures. If PRES is suspected, confirmatory magnetic resonance imaging (MRI) is performed.

Gastrointestinal involvement: Participants are assessed for signs and symptoms of GI involvement (e.g., diarrhea, vomiting, pain, and bleeding). The frequency and estimated volume of GI bleeding is recorded in the participant's CRF.

A complete physical examination includes, at a minimum, assessments of the following organs/body systems: skin, head, ears, eyes, nose, throat, neck, lymph nodes, chest, heart, abdomen, extremities, musculoskeletal, and neurological state.

An abbreviated physical examination includes, at a minimum, a body-system relevant examination based upon Investigator judgment and participant symptoms.

Investigators pay special attention to clinical signs related to previous serious illnesses. Additional physical examinations are performed as medically indicated during the study at the Investigator's discretion. Height and weight are measured and recorded.

Vital signs are monitored. Oral temperature (° C. or ° F.), heart rate, respiratory rate, systolic and diastolic blood pressure (mm Hg), and pulse oximetry are assessed. Blood pressure and heart rate measurements are assessed with the participant in a seated position using a completely automated device. Manual techniques are used only if an automated device is not available. Blood pressure and heart rate measurements are preceded by at least 5 minutes of rest for the participant in a quiet setting without distractions (e.g., television, cell phones). Ideally, the same arm for each participant is used for measurements. 02 saturation (%) is collected using pulse oximetry. Vital signs are collected predose and postdose at ravulizumab dosing visits.

Single 12-lead ECGs are conducted to obtain heart rate, PR, QRS, QT, and QTc intervals. (QT interval is corrected for heart rate using Fridericia's formula [QTcF].) Participants must be supine for approximately 5 to 10 minutes before ECG collection and remain supine but awake during ECG collection. The Investigator or Sub-Investigator are responsible for reviewing the ECG to assess whether the ECG is within normal limits and determine the clinical significance of the results. These assessments are recorded in the source documents and the CRF.

Both posteroanterior and lateral chest X-rays are obtained while the participant is at full inspiration at the timepoints specified in the SoA. The Investigator or designee Sub Investigator are responsible for reviewing the X-rays to assess the clinical significance of the results. These assessments are recorded in the source documents and the CRF. Particular attention is paid to determine whether signs of pulmonary or cardiovascular involvement are present (including, but not limited to, pulmonary hypertension, pleural effusion, and pulmonary edema).

Transthoracic echocardiography is performed as per the Schedule of Activities. The Investigator or designee Sub-Investigator are responsible for reviewing the echocardiogram to assess the clinical significance of the results. These assessments are recorded in the source documents and the CRF. Particular attention is paid to determine whether signs of pulmonary hypertension (right ventricular dysfunction, tricuspid regurgitation) or serositis (pericardial effusion) are present.

If a participant has symptoms of PRES at study entry or develops symptoms during the study, an MRI is performed to look for bilateral white matter abnormalities in the vascular watershed areas consistent with PRES. If the presence of PRES is confirmed, MRIs is performed upon resolution of symptoms and at the EoS/ED Visit. Additional MRIs can be performed during the study.

The Investigator must review the laboratory report, document this review, and record any clinically relevant changes occurring during the study in the AE section of the CRF. The laboratory reports must be filed with the source documents. Clinically significant abnormal laboratory findings are those which are not associated with the underlying disease, unless judged by the Investigator to be more severe than expected for the participant's condition.

All laboratory tests with values considered clinically significantly abnormal during participation in the study or within 8 weeks after the final dose of study intervention are repeated until the values return to normal or baseline or are no longer considered clinically significant by the Investigator or Medical Monitor. If such values do not return to normal/baseline within a period of time judged reasonable by the Investigator, the etiology is identified. All protocol-required laboratory assessments are conducted in accordance with the laboratory manual and the Schedule of Activities. I

Human immunodeficiency virus testing for HIV-1 and HIV-2 is required of all participants prior to enrollment. A documented history of negative HIV-1 and HIV-2 tests within 6 months prior to Screening is sufficient. Participants who are HIV positive are not enrolled.

Pregnancy testing is be performed as described in the Schedule of Assessments.

Prior medications and/or vaccines (including vitamins, herbal preparations, and those discussed in the exclusion criteria and procedures (any therapeutic intervention, such as surgery/biopsy or physical therapy) that the participant receives or undergoes within 30 days prior to Day 1 (Stage 1) or within 30 days prior to randomization (Stage 2) are recorded. Please refer to the inclusion/exclusion criteria for prior medication considerations for eligibility. In addition, use of certain medication is not allowed during the study. Information regarding the HSCT procedure is recorded on the HSCT information CRF page.

Prior medications and procedures are recorded in the participant's source document/medical chart and CRF along with: reason for use (indication), dates of administration including start and end dates (if the participant is expected to continue on the medication during the study, it should be recorded as continuing), and dosage information including dose and frequency.

Concomitant medications (including any medication, vitamin, herbal preparation or supplement) and procedures are those received on or after Day 1 (for Stage 1), after randomization for participants in the BSC-only treatment group (Stage 2) or after the start of ravulizumab IV infusion for participants in the ravulizumab plus BSC treatment group (Stage 2). This includes any supportive care medications and procedures, antibiotic prophylaxis, GVHD prophylaxis or treatment, and vaccinations. At each study visit, participants are questioned about any new medication or nondrug therapies or changes to concomitant medications and nondrug therapies since the last visit. Concomitant medications are recorded in the participant's source document/medical chart and CRF along with: reason for use (indication), dates of administration including start and end dates (if the participant is expected to continue on the medication during the study, it should be recorded as continuing), and dosage information including dose and frequency.

Any concomitant medication deemed necessary for the participant's care during the study, or for the treatment of any AE, along with any other medications, other than those listed as disallowed medications, may be given at the discretion of the Investigator. However, it is the responsibility of the Investigator to ensure that details regarding all medications are recorded in full in the participant's source document/medical chart and CRF page.

Data regarding transfusion of blood components is recorded on the transfusion CRF page, including date of transfusion, type of blood component administered, number of units and volume.

### 8. Adverse Events

An adverse event (AE) is any untoward medical occurrence in a participant, temporally associated with the use of study intervention, whether or not considered related to the study intervention. An AE can therefore be any unfavorable and unintended sign (including an abnormal laboratory finding), symptom, or disease (new or exacerbated) temporally associated with the use of study intervention. Events that meet the AE definition are set forth in Table 9. Events that do not meet the AE definition are set forth in Table 10.

If an event is not an AE per definition above, then it cannot be a Serious Adverse Event (SAE) even if serious conditions are met (e.g., hospitalization for signs/symptoms of the disease under study, death due to progression of disease). An SAE is defined as any untoward medical occurrence that, at any dose results in one of the events set forth in Table 11.

A suspected unexpected serious adverse reaction (SUSAR) is defined as a serious event that is not listed in the Investigator's Brochure and that the Investigator identifies as related to investigational product or procedure. United States Title 21 Code of Federal Regulations (CFR) 312.32 and European Union Clinical Trial Directive 2001/20/EC and the associated detailed guidances or national regulatory requirements in participating countries require the reporting of SUSARs. Suspected unexpected serious adverse reactions are reported to the national competent authority and IRBs/IECs where applicable.

All AEs are reported to the Investigator or qualified designee by the participant (or, when appropriate, by a caregiver, surrogate, or the participant's legally authorized representative). The Investigator and any qualified designees are responsible for detecting, documenting, and recording events that meet the definition of an AE or SAE and remain responsible for following up AEs that are serious, considered related to the study intervention or study procedures, or that caused the participant to discontinue the study intervention.

All AEs and SAEs are collected from the signing of the ICF/Assent Form until the ED/EoS Visit or 8 weeks after the final dose of ravulizumab (whichever occurs later). All SAEs are recorded and reported immediately and under no circumstance should this exceed 24 hours. Investigators are not obligated to actively seek AE or SAE data after conclusion of the study participation.

For recording and follow-up of an AE and/or SAE, the intensity is assessed. An event is defined as “serious” when it meets at least one of the predefined outcomes as described in the definition of an SAE, not when it is rated as severe. The Investigator makes an assessment of intensity for each AE and SAE reported during the study and assign it to one of the following categories from National Cancer Institute CTCAE v5.0, published 27 Nov. 2017: (a) Grade 1: Mild (awareness of sign or symptom, but easily tolerated), (b) Grade 2: Moderate (discomfort sufficient to cause interference with normal activities), (c) Grade 3: Severe (incapacitating, with inability to perform normal activities), (d) Grade 4: Life-threatening, or (e) Grade 5: Fatal.

The Investigator is obligated to assess the relationship between the study intervention and each occurrence of each AE or SAE. An Investigator causality assessment must be provided for all AEs (both nonserious and serious). This assessment must be recorded in the CRF and on any additional forms, as appropriate. The definitions for the causality assessments are as follows:
(a) Not related: There is no reasonable possibility the study intervention caused the AE. The AE has a more likely alternative etiology; it may be due to underlying or concurrent illness, complications, concurrent treatments, or effects of another concurrent drug. The event does not follow a reasonable temporal relationship to administration of the study intervention
(b) Related: There is a reasonable possibility the study intervention caused the AE. The AE has a temporal relationship to the administration of the study intervention. The event does not have a likely alternative etiology. The event corresponds with the known pharmaceutical profile of the study intervention. There is improvement on discontinuation and/or reappearance on rechallenge.
   The Investigator uses clinical judgment to determine the relationship. Alternative causes, such as underlying disease(s), concomitant therapy, and other risk factors, as well as the temporal relationship of the event to study intervention administration are considered and investigated. The Investigator also consults the Investigator's Brochure (IB) and/or Product Information, for marketed products, in his/her assessment. For each AE/SAE, the Investigator must document in the medical notes that he/she has reviewed the AE/SAE and has provided an assessment of causality. There may be situations in which an SAE has occurred, and the Investigator has minimal information to include in the initial report to Alexion. However, it is very important that the Investigator always make an assessment of causality for every event before the initial transmission of the SAE data to Alexion. The Investigator may change his/her opinion of causality in light of follow-up information and send an SAE follow-up report with the updated causality assessment. The causality assessment is one of the criteria used when determining regulatory reporting requirements.

### 9. Pharmacokinetics and Pharmacodynamics

Blood samples are collected for determination of serum ravulizumab, free C5, and total C5 concentrations at the timepoints specified in the Schedule of Activities. The timing of sampling may be altered during the course of the study, based on newly available data (e.g., to obtain data closer to the time of peak plasma concentrations) to ensure appropriate monitoring. Samples collected for PK/PD analyses can also be used to evaluate safety or efficacy aspects related to concerns arising during or after the study.

Instructions for the collection and handling of biological samples, including blood volume requirements, are provided in the Laboratory Manual. The actual date and time (24-hour clock time) of each sample is recorded.

For all participants in Stage 1, participants in Stage 2 randomized to the ravulizumab plus BSC treatment group, participants receiving rescue ravulizumab therapy, and participants receiving retreatment with ravulizumab: Predose PK and PD blood samples are collected within 90 minutes before administering ravulizumab at visits specified in the Schedule of Assessments. The predose blood sample can be drawn through the venous access created for the dose infusion, prior to administration of the dose. Postdose PK and PD blood samples are collected within 60 minutes after completing ravulizumab infusion. The postdose blood samples are drawn from the participant's opposite, non-infused arm. PK/PD blood samples at a non-dosing visit can be collected at any time during the visit.

For participants randomized to the BSC-only treatment group in Stage 2: PD blood samples (free C5 and total C5) can be collected at any time at the visits specified in the Schedule of Assessments. In the event of an unscheduled visit, PK and PD blood samples are collected as soon as possible. Predose and postdose PK and PD samples are collected for all supplemental doses of ravulizumab administered.

### 10. Genetics

For participants who sign an additional optional consent, whole blood and buccal swab samples for exploratory genetics are collected at the time point specified in the Schedule of Assessments. Exploratory genetics can be performed to investigate genetic variants in genes that may be associated with complement dysregulation or metabolism or efficacy of ravulizumab.

### 11. Biomarkers

Blood and urine samples for biomarker research are collected from all participants at the time points specified in the Schedule of Assessments. Whenever possible, collection of biomarker samples during the study, including samples collected during the Screening Period, are performed prior to the participant receiving transfusions or dialysis.

Biomarkers measured and include, but are not limited to, assessments of the following: (a) Vascular inflammation (e.g., shed tumor necrosis factor receptor 1 [TNF-R1]) (b) Endothelial damage and/or activation (e.g., thrombomodulin and shed vascular cell adhesion molecule 1 [VCAM-1]), renal injury (e.g., Cystatin C), and (d) Complement proteins and complement activation pathway products

### 12. Immunogenicity Assessments

Antidrug antibodies are evaluated in serum samples collected predose (within 90 minutes prior to the start of ravulizumab IV infusion) from all participants at time points specified in the Schedule of Assessment. Additionally, serum samples are also collected at the final visit from participants who discontinued ravulizumab or were withdrawn from the study.

Serum samples are screened for antibodies binding to ravulizumab and the titer of confirmed positive samples will be reported. Other analyses can be performed to further characterize the immunogenicity of ravulizumab.

The detection and characterization of antibodies to ravulizumab is performed using a validated assay method. Samples collected for detection of antibodies to ravulizumab are also evaluated for ravulizumab serum concentration to enable interpretation of the antibody data. Confirmed antibody positive samples are further evaluated for antibody titer and the presence of neutralizing antibodies.

### 13. Exploratory Assessments

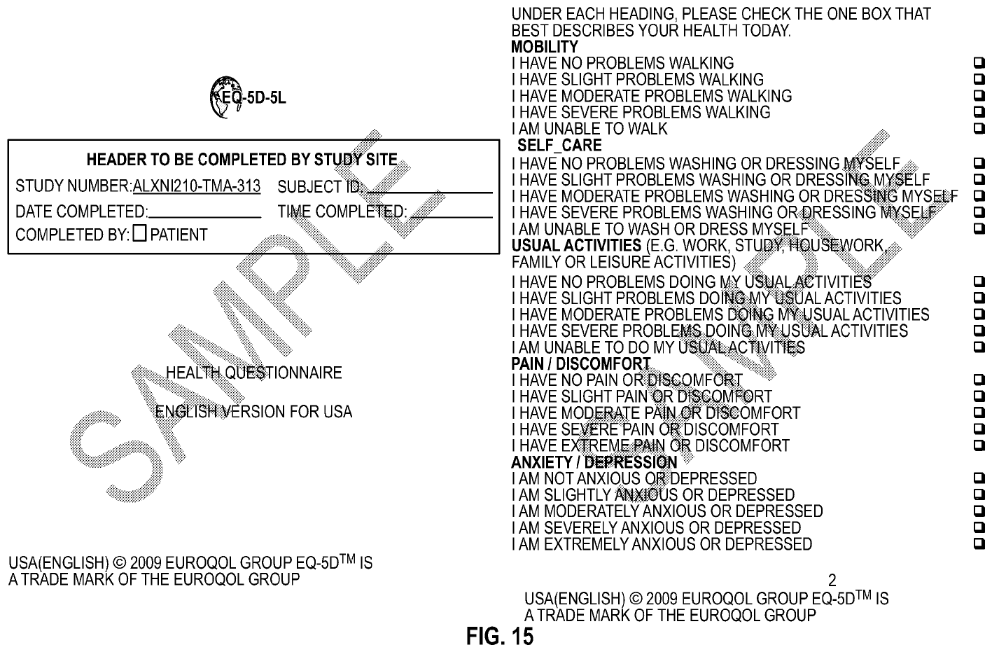

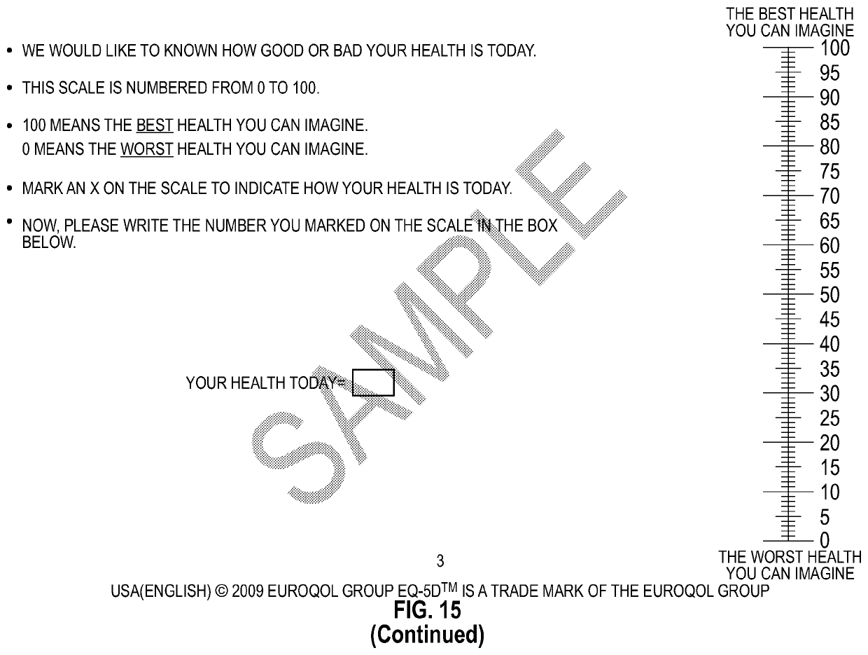

The EuroQoL 5-Dimensions 5-Level (EQ-5D-5L) is a self-assessed, standardized instrument to measure health related quality of life (QoL) and has been used in a wide range of health conditions. A sample Qol is set forth in FIG. 15. The EQ 5D 5L is administered electronically by the Investigator or a qualified site staff prior to other study procedures at visits specified in the Schedule of Activities.

### 14. Statistical Considerations

For the primary analysis, the ravulizumab plus BSC treatment group is compared to the BSC-only treatment group based on the null and alternative hypotheses below. Hypothesis testing are two-sided and performed at the 0.05 level of significance. The null and alternative hypotheses for the primary analysis are as follows:

H₀:P_{rav+BSC}=P_{BSC} vs. H_{A}:P_{rav+BSC}≠P_{BSC}

where P_{rav+BSC} is the proportion of participants achieving TMA response in the ravulizumab plus BSC treatment group and P_{BSC} is the proportion of participants achieving TMA response in the BSC-only treatment group.

The primary analysis is based on a 2-sided Fisher's exact test performed at a 5% significance level, comparing the proportion of participants achieving TMA response during the 26-week Treatment Period in participants randomized to ravulizumab plus BSC treatment versus participants randomized to BSC treatment only. A sample size of 174 (87 participants per treatment group) has 90% power to detect a statistically significant (p≤0.05) treatment difference of 25 percentage points in the proportion of responders at a two-sided significance level of 0.05, assuming the responder rate is 30% with BSC-only and 55% with ravulizumab plus BSC.

The analysis sets are defined in Table 19. These definitions for analysis sets are applied for participants enrolled in both Stage 1 and Stage 2 of the study. However, data from each stage is analyzed and reported separately, except for selected safety analyses which can combine results from both stages.

TABLE 19
Analysis Sets

| Analysis set | Description |
|---|---|
| Enrolled Set | All consented participants meeting eligibility |
| | criteria. |
| Safety Set | All participants who receive at least 1 dose of |
| | ravulizumab or receive BSC treatment. |
| Full Analysis Set | All participants who receive at least 1 dose of |
| | ravulizumab or receive BSC treatment. |
| Pharmacokinetic | All participants who receive at least 1 dose of |
| and | ravulizumab and who have evaluable |
| Pharmacodynamic | pharmacokinetic or pharmacodynamic data. |
| Analysis Set | |
| Per Protocol Set | Defined in the statistical analysis plan. |

Abbreviation: BSC = best supportive care.

Efficacy analyses are performed on the Full Analysis Set (FAS). The primary efficacy endpoint analysis, as well as selected secondary endpoint analyses, are also performed on the Per Protocol Set. Safety analyses are performed on the Safety Set. Pharmacokinetic and PD analyses are performed on all participants who receive at least 1 dose of ravulizumab and who have evaluable PK or PD data.

Statistical methods described in this section are further elaborated in a separate statistical analysis plan (SAP). Summary statistics are computed and displayed by treatment group, and by visit, where applicable, and by stratification factors, when relevant. Descriptive statistics for continuous variables minimally include the number of participants, mean, standard deviation, minimum, median, and maximum. For categorical variables, frequencies, and percentages are presented. Graphical displays are provided as appropriate.

Analyses are performed using the SAS® software Version 9.4 or higher. The main set of analyses focuses on comparing results between the 2 treatment groups. However, if a large proportion of participants in the BSC-only treatment group receives rescue ravulizumab, a comparison between treatment groups may not be meaningful for secondary endpoints. Participants who receive rescue therapy initiate a new visit schedule from the time of the start of their rescue therapy. This visit schedule is equivalent to the one for participants randomized to the ravulizumab plus BSC treatment group at the start of the study. The data collected from the start of rescue therapy is summarized separately, allowing for evaluation of TMA response (irrespective of the early assignment of non-response used for the primary endpoint analysis) and other endpoints among these participants. Analyses that present or are based on changes from baseline for participants who receive rescue therapy have the baseline value assigned as the value obtained from the last assessment prior to the start of rescue therapy.

Additionally, a set of analyses of efficacy endpoints is conducted without consideration for early assignment of non-response and rescue therapy, simply presenting results by randomized treatment group even for data obtained after early assignment of non-response or the initiation of rescue therapy. Analyses of the primary endpoint is performed by subgroups including, but not limited to the presence of GVHD, geographical region, and baseline weight.

The primary efficacy endpoint is TMA response during the initial 26-week randomized Treatment Period. Participants must meet each TMA response criterion at 2 separate assessments obtained at least 28 days apart, and any measurement in between; and all intervals during which each criterion is met must overlap for at least one day to be classified as having met the primary efficacy endpoint.

The proportion of participants who achieve TMA response during the 26-week randomized Treatment Period is summarized for each treatment group. The primary analysis is based on a 2-sided Fisher's exact test performed at a 5% significance level, comparing the proportion of participants achieving TMA response during the 26-week randomized Treatment Period in participants randomized to ravulizumab plus BSC treatment versus participants randomized to BSC-only treatment.

The analysis is performed based on the randomized treatment assignment. To avoid bias and confounding of results that could stem from the available rescue therapy for participants randomized to the BSC-only group, only participants assigned as non-responders through early non-response assignment are eligible for such rescue therapy. The early non response assignment is applied in the same manner to both treatment groups, even if rescue therapy is only available to the BSC-only group. Participants assigned as early non responders are classified as non-responders for the primary endpoint of TMA response during the 26-week randomized Treatment Period irrespective of results that may be observed following the early assignment of non-response.

Among participants who receive rescue therapy, the proportion of participants who achieve TMA response during the 26-week Treatment Period from the start of rescue therapy are summarized. The early assignment of non-response required to receive rescue therapy are not considered for this analysis. Only data collected from the start of rescue therapy is used to determine TMA response.

For time to TMA response, participants are assigned as responders at the time of their TMA response and are censored at the earliest of their early non-response assignment time, discontinuation time, or at the end of available follow-up if they have not responded by then. Cumulative incidence of TMA response is estimated using competing risk survival analysis methods to account for death as a competing risk. Point estimates and 95% CIs are provided.

This analysis will be repeated among participants who receive rescue therapy using only data from the start of rescue therapy. This analysis is also repeated without consideration for early assignment of non-response and rescue therapy, simply presenting results by randomized treatment group even for data obtained after early assignment of non-response or the initiation of rescue therapy.

In addition to the primary analysis of TMA response during the 26-week randomized Treatment Period, TMA response is also summarized by treatment group over time by presenting the number and proportion of responders along with a 2-sided 95% CI for each postbaseline time point. This analysis is conducted using 2 different approaches. The first one presents the number and proportion of responders at a specific time point out of the participants who are still in the study up to this time point. The second approach presents the cumulative number and proportion of responders up to a specific time point out of all participants randomized. This analysis includes time points of importance such as Week 26 and 52. This analysis is repeated among participants who receive rescue therapy using only data from the start of rescue therapy. This analysis is also be repeated without consideration for early assignment of non-response and rescue therapy, simply presenting results by randomized treatment group even for data obtained after early assignment of non-response or the initiation of rescue therapy.

Response to each individual component of TMA response during the 26 week randomized Treatment Period is summarized by treatment group by calculating the point estimate and a 95% CI for the proportion of responders. The CI is based on exact confidence limits using the Clopper-Pearson method. These responses are also summarized by treatment group over time by presenting the number and proportion of responders along with a 2-sided 95% CI for each postbaseline time point. This analysis is conducted using 2 different approaches. The first one presents the number and proportion of responders at a specific time point out of the participants who are still in the study up to this time point. The second approach presents the cumulative number and proportion of responders up to a specific time point out of all participants randomized. This analysis is repeated among participants who receive rescue therapy using only data from the start of rescue therapy. This analysis is also be repeated without consideration for early assignment of non-response and rescue therapy, simply presenting results by randomized treatment group even for data obtained after early assignment of non-response or the initiation of rescue therapy.

Time to response for each criterion of TMA response is assessed separately. Participants are assigned as responders for a specific criterion at the time of their response for that criterion and are censored at the time of their early non-response assignment, discontinuation time or at the end of available follow-up if they have not responded by then. Cumulative incidence of the response criterion of interest is estimated using competing risk survival analysis methods to account for death as a competing risk. Point estimates and 95% CIs will be provided. This analysis is repeated among participants who receive rescue therapy using only data from the start of rescue therapy. This analysis is also repeated without consideration for early assignment of non-response and rescue therapy, simply presenting results by randomized treatment group even for data obtained after early assignment of non-response or the initiation of rescue therapy.

Participants must meet each response criterion at 2 separate assessments obtained at least 28 days apart, and any measurement in between; and all intervals during which each criterion is met must overlap for at least one day to be classified as having met this efficacy endpoint. Hematologic response during the 26-week randomized Treatment Period is summarized by treatment group by calculating the point estimate and a 95% CI for the proportion of responders. The CI is based on exact confidence limits using the Clopper-Pearson method.

Response is also summarized by treatment group over time by presenting the number and proportion of responders along with a 2-sided 95% CI for each postbaseline time point. This analysis is conducted using 2 different approaches. The first one presents the number and proportion of responders at a specific time point out of the participants who are still in the study up to this time point. The second approach presents the cumulative number and proportion of responders up to a specific time point out of all participants randomized. This analysis is repeated among participants who receive rescue therapy using only data from the start of rescue therapy. This analysis is also repeated without consideration for early assignment of non-response and rescue therapy, simply presenting results by randomized treatment group even for data obtained after early assignment of non-response or the initiation of rescue therapy.

For time to hematologic response, participants are assigned as responders at the time of their response and are censored at the earliest of their early non-response assignment time, discontinuation time or at the end of available follow-up if they have not responded by then. Cumulative incidence of hematologic response is estimated using competing risk survival analysis methods to account for death as a competing risk. Point estimates and 95% CIs are be provided. This analysis is repeated among participants who receive rescue therapy using only data from the start of rescue therapy. This analysis is also repeated without consideration for early assignment of non-response and rescue therapy, simply presenting results by randomized treatment group even for data obtained after early assignment of non-response or the initiation of rescue therapy.

The following criterion must be met for hemoglobin response: Hemoglobin ≥8 g/dL. Participants must meet this criterion at 2 separate assessments obtained at least 28 days apart, and any measurement in between, and they should not receive transfusion during this period. Hemoglobin maintenance during the 26-week randomized Treatment Period is summarized by treatment group by calculating the point estimate and a 95% CI for the proportion of responders. The CI is based on exact confidence limits using the Clopper-Pearson method.

Response is also summarized by treatment group over time by presenting the number and proportion of responders along with a 2-sided 95% CI for each postbaseline time point. This analysis is conducted using 2 different approaches. The first one presents the number and proportion of responders at a specific time point out of the participants who are still in the study up to this time point. The second approach presents the cumulative number and proportion of responders up to a specific time point out of all participants randomized. This analysis is repeated among participants who receive rescue therapy using only data from the start of rescue therapy. This analysis is also repeated without consideration for early assignment of non-response and rescue therapy, simply presenting results by randomized treatment group even for data obtained after early assignment of non-response or the initiation of rescue therapy.

Hematologic parameters (hemoglobin, platelets, LDH, and schistocytes) are summarized by treatment group at baseline and each postbaseline time point using descriptive statistics for continuous variables for the observed value as well as the change from baseline. This analysis is repeated among participants who receive rescue therapy using only data from the start of rescue therapy. This analysis is also repeated without consideration for early assignment of non-response and rescue therapy, simply presenting results by randomized treatment group even for data obtained after early assignment of non-response or the initiation of rescue therapy.

Partial TMA response (participant meets ≥1, but not all, criteria for TMA response [Section 3]) during the 26-week Treatment Period is summarized by treatment group by calculating the point estimate and a 95% CI for the proportion of responders. The CI is based on exact confidence limits using the Clopper-Pearson method. This includes separate presentations for participants with response to only 1 component, at least 1, but not all components, only 2 components, and at least 2, but not all components. Response is also summarized by treatment group over time by presenting the number and proportion of responders along with a 2-sided 95% CI for each postbaseline time point. This analysis is repeated among participants who receive rescue therapy using only data from the start of rescue therapy. This analysis is also be repeated without consideration for early assignment of non-response and rescue therapy, simply presenting results by randomized treatment group even for data obtained after early assignment of non-response or the initiation of rescue therapy.

Treatment effect on organ dysfunction will be evaluated in the following end organ systems: renal, cardiovascular, CNS, pulmonary, and GI. The following analyses are repeated among participants who receive rescue therapy using only data from the start of rescue therapy.

Kidney function parameters (protein/creatinine ratio, serum creatinine, and eGFR) are summarized by treatment group at baseline and each postbaseline time point using descriptive statistics for continuous variables for the observed value as well as the change from baseline. For participants who require dialysis, change from baseline in dialysis requirements is summarized. An analysis presents the number and proportion of participants who require dialysis over time. A 2-sided 95% CI for the proportion is provided. A participant is considered as not requiring dialysis at a specific postbaseline time point if they have been dialysis free for at least 5 days prior to that time point.

The presence of pulmonary hypertension, pleural effusion, pulmonary edema, and pericardial effusion is summarized. An analysis presents over time the number and proportion of participants with any of these conditions. A 2-sided 95% CI for the proportion is provided. Additionally, use of any ventilatory or respiratory support is summarized similarly.

Hypertension is assessed by blood pressure measurement at every visit as part of vital signs collection, and analysis of concomitant medications to control the hypertension. Presence of hypertension is summarized by presenting the number and proportion of participants who shift in their hypertensive status from baseline to post baseline visits. Selected echocardiography parameters will be summarized over time.

The presence of PRES is summarized by presenting the number and proportion of participants with shifts in presence of PRES status from baseline to post baseline visits.

Signs and symptoms of GI involvement such as diarrhea, vomiting, pain, and bleeding are summarized. An analysis presents over time the number and proportion of participants with any of these conditions. The frequency and estimated volume of GI bleeding is also summarized.

Among participants who have achieved TMA response, TMA relapse (participant meets the criteria for TMA diagnosis after achieving TMA response) is summarized by treatment group by calculating the point estimate and a 95% CI for the proportion of participants with a TMA relapse. The CI is based on exact confidence limits using the Clopper-Pearson method.

An analysis summarizes time to TMA relapse from the time of response. Participants are censored at the time of the end of their available follow-up if they have not experienced a relapse. Kaplan Meier cumulative distribution curves are generated for each treatment group. A corresponding summary table presents the first quartile, median, and third quartile, along with corresponding 2-sided 95% CI, of time to TMA relapse. This analysis is repeated among participants who receive rescue therapy using only data from the start of rescue therapy. This analysis is also repeated without consideration for early assignment of non-response and rescue therapy, simply presenting results by randomized treatment group even for data obtained after early assignment of non-response or the initiation of rescue therapy.

Loss of response occurs when a participant achieves TMA response and fails to meet the criteria for at one or more components of TMA response at a subsequent visit (must be confirmed by a second laboratory result at least 24 hours apart). Participants who achieve loss of response are summarized by treatment group by calculating the point estimate and a 95% CI for the proportion of participants with a loss of TMA response. The CI is based on exact confidence limits using the Clopper-Pearson method. Duration of response is assessed with an analysis of time to loss of TMA response from the time of response. Participants are censored at the time of the end of their available follow-up if they have not experienced a loss of response. Kaplan Meier cumulative distribution curves are generated for each treatment group. A corresponding summary table presents the first quartile, median, and third quartile, along with corresponding 2-sided 95% CI, of time to loss of TMA response. This analysis is repeated among participants who receive rescue therapy using only data from the start of rescue therapy. This analysis is also be repeated without consideration for early assignment of non-response and rescue therapy, simply presenting results by randomized treatment group even for data obtained after early assignment of non-response or the initiation of rescue

Survival time is assessed as the number of days from randomization to an event of death. Participants who survive are censored at their last available follow-up or when they initiate rescue therapy. Kaplan-Meier cumulative distribution curves are generated for each treatment group. A corresponding summary table presents by treatment group the first quartile, median, and third quartile, along with corresponding 2-sided 95% CI, of survival time. This analysis is repeated among participants who receive rescue therapy using only data from the start of rescue therapy. This analysis is also repeated without consideration for early assignment of non-response and rescue therapy, simply presenting results by randomized treatment group even for data obtained after early assignment of non-response or the initiation of rescue therapy.

Non-relapse mortality is defined as participant's death due to any cause during the study, with the exception of death due to underlying disease progression or relapse. Cumulative incidence of non-relapse deaths is estimated using competing risk survival analysis methods to account for relapse-related deaths as a competing risk. Point estimates and 95% CIs are provided. Subjects who do not experience a non-relapse death are right censored at their last available follow-up or when they initiate rescue therapy. This analysis is repeated among participants who receive rescue therapy using only data from the start of rescue therapy. This analysis is also repeated without consideration for early assignment of non-response and rescue therapy, simply presenting results by randomized treatment group even for data obtained after early assignment of non-response or the initiation of rescue.

The following criterion must be met for platelet response: Platelet count ≥100,000/mm3 without transfusion support during the prior 7 days. Participants must meet this criterion at 2 separate assessments obtained at least 28 days apart, and any measurement in between, and they do not receive transfusion during this period. Maintenance of platelet count ≥100,000/mm3 during the 26-week randomized Treatment Period is summarized by treatment group by calculating the point estimate and a 95% CI for the proportion of responders. The CI is based on exact confidence limits using the Clopper Pearson method. Response is also summarized by treatment group over time by presenting the number and proportion of responders along with a 2-sided 95% CI for each postbaseline time point. This analysis is conducted using 2 different approaches. The first one presents the number and proportion of responders at a specific time point out of the participants who are still in the study up to this time point. The second approach presents the cumulative number and proportion of responders up to a specific time point out of all participants randomized. This analysis is repeated among participants who receive rescue therapy using only data from the start of rescue therapy. This analysis is also repeated without consideration for early assignment of non-response and rescue therapy, simply presenting results by randomized treatment group even for data obtained after early assignment of non-response or the initiation of rescue therapy.

For the primary analysis, the ravulizumab plus BSC treatment group is compared to the BSC-only treatment group based on the null and alternative hypotheses below. Hypothesis testing is two-sided and performed at the 0.05 level of significance. The null and alternative hypotheses for the primary analysis are as follows:

$${H}_{0} : {P}_{r a v + B S C} = {P}_{BSC} vs . {H}_{A} : {P}_{r a v + B S C} \neq {P}_{BSC}$$

where P_{rav+BSC} is the proportion of participants achieving TMA response in the ravulizumab plus BSC treatment group and PBSC is the proportion of participants achieving TMA response in the BSC-only treatment group. If the null hypothesis is rejected, key secondary endpoints are tested for inequality using a hierarchical stepdown closed-testing procedure. The secondary endpoints are tested in the following order until a non-significant test is observed, at which point no further testing of subsequent endpoints will occur: (1) time to TMA response for randomized treatment assignment, (2) hematologic response for randomized treatment assignment, (3) overall survival for randomized treatment assignment, (4) non-relapse mortality for randomized treatment assignment, and (5) change from baseline in eGFR for randomized treatment assignment.

For exploratory biomarker analyses, summary statistics are presented for observed values, change and percentage change from baseline. The relationship between ravulizumab concentration and exploratory biomarkers or the correlation between clinical benefit and key exploratory biomarkers can be assessed by graphical display. Exploratory analysis and potential relationships between clinical outcomes, PK/PD, genetic profile, and biomarker levels can also performed and results summarized if evaluated. Quality of life is evaluated using EQ-5D-5L. The visual assessment scale score is obtained and the US composite time trade-off calculated based on this instrument and these are summarized at baseline and each postbaseline time point using descriptive statistics for continuous variables for the observed value as well as the change from baseline. This analysis is repeated among participants who receive rescue therapy using only data from the start of rescue therapy. This analysis is also repeated without consideration for early assignment of non-response and rescue therapy, simply presenting results by randomized treatment group even for data obtained after early assignment of non-response or the initiation of rescue therapy.

All safety analyses are performed on the Safety Set and based on the treatment received. Participants who receive rescue therapy will have their BSC and rescue therapy data summarized separately. The following definitions are used for AEs:

TABLE 20
AE Definitions

Pretreatment adverse events: Any AE that starts after providing informed
consent/assent, but before randomization (BSC-only treatment group) or
before the first infusion of ravulizumab (ravulizumab plus BSC treatment
group).
Treatment-emergent adverse event (TEAE): Any AE that starts during or
after randomization (BSC-only treatment group) or after the start of the
first infusion of ravulizumab (ravulizumab plus BSC treatment group).
Treatment-emergent SAE (TESAE): A TEAE that is serious.

The incidence of TEAEs, TEAEs leading to withdrawal from the study, TEAEs leading to study treatment discontinuation, drug-related TEAEs, TEAEs during ravulizumab administration, and SAEs are summarized by treatment group. All AEs are coded using the Medical Dictionary for Regulatory Activities (MedDRA) Version 22 or higher and are summarized by System Organ Class (SOC) and Preferred Term overall, by severity, and by relationship to treatment. Detailed by-participant listings of TEAEs, SAEs, related TEAEs, TEAEs during ravulizumab administration, TEAEs leading to withdrawal from the study, and TEAEs leading to study treatment discontinuation are provided.

Adverse changes from baseline in physical examination findings are classified as AEs and analyzed accordingly. Vital signs are summarized descriptively at baseline and postbaseline time points and for changes from baseline.

Observed values and changes from baseline in clinical chemistry, hematology, and urinalysis are summarized descriptively at baseline, and at each postbaseline time point. For laboratory results that can be classified as normal, low or high based on normal range values, shifts from baseline in classification are summarized for all study visits. Protocol-required safety laboratory assessments are set forth in Table 20.

TABLE 20
Protocol-Required Safety Laboratory Assessments

| Laboratory | |
|---|---|
| Assessments | Parameters |
| Hematology | Platelet count |
| | Immature platelet fraction |
| | Red blood cell count |
| | Hemoglobin (including free hemoglobin) |
| | Hematocrit |
| | RBC Indices (mean corpuscular volume, mean |
| | corpuscular hemoglobin,) |
| | Percentage of reticulocytes |
| | RBC distribution width |
| | White blood cell count with differential (including |
| | early progenitors) |
| | Haptoglobin |
| | RBC morphology (peripheral blood smear); |
| | particularly the presence of schistocytes |
| Coagulation | INR |
| Panel | PT |
| | APTT |
| | D-Dimer |
| | Fibrinogen |
| Clinical | Liver function tests: |
| Chemistry^{a} | ALT |
| | AST |
| | ALP |
| | Albumin |
| | total protein |
| | A/G ratio (albumin/globulin ratio) |
| | Bilirubin (total, direct and indirect) |
| | GGT |
| | Glucose (non-fasting) |
| | LDH |
| | Renal function: |
| | Anion gap |
| | Bicarbonate |
| | Blood urea nitrogen |
| | Calcium |
| | Chloride |
| | Creatinine |
| | Magnesium |
| | Phosphorus |
| | Potassium |
| | Sodium |
| | Uric acid |
| | Other clinical chemistry parameters: |
| | Amylase |
| | Creactive protein |
| | Lipase |
| | Troponin I |
| | Von Willebrand Factor |
| Routine | Albumin |
| Urinalysis/ | Bilirubin |
| Urine | Blood |
| Chemistry | Color |
| | Creatinine |
| | Glucose |
| | Ketones |
| | Nitrite |
| | pH |
| | Protein |
| | Specific gravity |
| | Urobilinogen |
| Other | Serum follicle-stimulating hormone and estradiol (as |
| Screening | needed in women of non-childbearing potential only) |
| Tests | Serum or urine human chorionic gonadotropin |
| | pregnancy test (as needed for WOCBP)^{a} |
| | HIV antibody (HIV-1and HIV-2) |
| | ADAMTS13 activity |
| | ST-HUS screen (eg, Shiga toxin enzyme |
| | immunoassay/PCR in stool/stool culture/rectal swab) |
| | Coombs test, direct |
| Other Study- | Immunogenicity (ADA) |
| specific Tests | Serum PK |
| | Serum PD (total and free C5) |
| | Soluble C5b-9 |
| | Genetic testing (whole blood and buccal swab) |
| | Exploratory biomarkers |

^{a}Serum pregnancy test at Screening and End of Study Visit/Early Discontinuation Visit, and local urine pregnancy test at all other times as specified in SoA
Abbreviations: ADAMTS13 = a disintegrin and metalloproteinase with a thrombospondin type 1 motif, member 13; ALP = alkaline phosphatase; ALT = alanine aminotransferase; APTT = activated partial thromboplastin time; AST = aspartate transaminase; C5 = complement component 5; GGT = gamma-glutamyltransferase; HIV = human immunodeficiency virus; INR = International normalized ratio; LDH = lactate dehydrogenase; PD = pharmacodynamics; PK = pharmacokinetics; PT = prothrombin time; ST-HUS = Shiga toxin-related hemolytic uremic syndrome; WOCBP = woman of childbearing potential.

By-participant data listings of ECG parameters are provided. Electrocardiograms are be evaluated and summarized as normal, abnormal not clinically significant, or abnormal clinically significant. A shift from baseline to worst on-study ECG table is presented for ECG results. Observed values and change from baseline in ECG intervals (PR, RR, QT, and QTc) are summarized descriptively at baseline and each postbaseline time point. QT interval will be corrected for heart rate using Fridericia's formula (QTcF).

The incidence and titers for ADAs to ravulizumab are presented at each postbaseline time point in tabular format. Additionally, any confirmed ADA positive samples are tested for antibody titer and the presence of neutralizing antibodies to ravulizumab.

Individual PK/PD data is collected for all participants. Graphs of mean serum ravulizumab concentration-time profiles are constructed. Graphs of serum concentration-time profiles for individual participants can also be provided. Actual dose administration and sampling times are used for all calculations. Descriptive statistics re calculated for serum concentration data at each sampling time, as appropriate. The PD effects of ravulizumab is evaluated by assessing the absolute values and changes and percentage changes from baseline in serum free C5 and serum total C5 concentrations over time, as appropriate. Descriptive statistics are calculated for the PD data at each sampling time, as appropriate.

An early analysis of the PK/PD data is initiated when the tenth participant completes Visit 5 on Day 21. All PK and free C5 data collected in all participants by that time is analyzed to confirm the adequacy of the initial dosing regimen to achieve complete inhibition of C5. If necessary, the dosing regimen is adjusted.

A primary endpoint analysis is conducted at the interim time point when all participants in Stage 2 have been followed for 26 weeks from randomization or withdraw from the initial 26 weeks of the study.

A separate analysis is conducted when all participants who received rescue therapy have discontinued from the study or completed the 26 weeks following initiation of ravulizumab treatment. Data since the initiation of ravulizumab treatment is considered for this analysis.

The final study analysis is conducted at the end of the study.

SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO : 6
QNVLNTPLT
SEQ ID NO: 7
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SS
SEQ ID NO: 8
DIQMTQSPSS LSASVGDRVT ITCGASENIY GALNWYQQKP GKAPKLLIYG
ATNLADGVPS RESGSGSGTD FTLTISSLQP EDFATYYCQN VLNTPLTFGQ
GTKVEIK
SEQ ID NO : 9
ASTKGPSVFP LAPCSRSTSE STAALGCLVK DYFPEPVTVS WNSGALTSGV
HTFPAVLQSS GLYSLSSVVT VPSSNFGTQT YTCNVDHKPS NTKVDKTVER
KCCVECPPCP APPVAGPSVF LFPPKPKDTL MISRTPEVTC VVVDVSQEDP
EVQFNWYVDG VEVHNAKTKP REEQFNSTYR VVSVLTVLHQ DWLNGKEYKC
KVSNKGLPSS IEKTISKAKG QPREPQVYTL PPSQEEMTKN QVSLTCLVKG
FYPSDIAVEW ESNGQPENNY KTTPPVLDSD GSFFLYSRLT VDKSRWQEGN
VFSCSVMHEA LHNHYTQKSL SLSLGK
SEQ ID NO: 10
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNEGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQFNST
YRVVSVLTVL HQDWLNGKEY KCKVSNKGLP SSIEKTISKA KGQPREPQVY
TLPPSQEEMT NQVSLTCLVK GFYPSDIAVE WESNGQPENN YKTTPPVLDS
DGSFFLYSRL TVDKSRWQEG NVFSCSVMHE ALHNHYTQKS LSLSLGK
SEQ ID NO: 11
DIQMTQSPSS LSASVGDRVT ITCGASENIY GALNWYQQKP GKAPKLLIYG
ATNLADGVPS RESGSGSGTD FTLTISSLQP EDFATYYCQN VLNTPLTFGQ
GTKVEIKRTV AAPSVFIFPP SDEQLKSGTA SVVCLLNNFY PREAKVQWKV
DNALQSGNSQ ESVTEQDSKD STYSLSSTLT LSKADYEKHK VYACEVTHQG
LSSPVTKSEN RGEC
SEQ ID NO: 12
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SS
SEQ ID NO: 13
ASTKGPSVFP LAPCSRSTSE STAALGCLVK DYFPEPVTVS WNSGALTSGV
HTFPAVLQSS GLYSLSSVVT VPSSNEGTQT YTCNVDHKPS NTKVDKTVER
KCCVECPPCP APPVAGPSVF LFPPKPKDTL MISRTPEVTC VVVDVSQEDP
EVQFNWYVDG VEVHNAKTKP REEQFNSTYR VVSVLTVLHQ DWLNGKEYKC
KVSNKGLPSS IEKTISKAKG QPREPQVYTL PPSQEEMTKN QVSLTCLVKG
FYPSDIAVEW ESNGQPENNY KTTPPVLDSD GSFFLYSRLT VDKSRWQEGN
VFSCSVLHEA LHSHYTQKSL SLSLGK
SEQ ID NO: 14
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNEGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQFNST
YRVVSVLTVL HQDWLNGKEY KCKVSNKGLP SSIEKTISKA KGQPREPQVY
TLPPSQEEMT KNQVSLTCLV KGFYPSDIAV EWESNGQPEN NYKTTPPVLD
SDGSFFLYSR LTVDKSRWQE GNVFSCSVLH EALHSHYTQK SLSLSLGK
SEQ ID NO: 15
ASTKGPSVFP LAPCSRSTSE STAALGCLVK DYFPEPVTVS WNSGALTSGV
HTFPAVLQSS GLYSLSSVVT VTSSNFGTQT YTCNVDHKPS NTKVDKTVER
KCCVECPPCP APPVAGPSVF LFPPKPKDTL YITREPEVTC VVVDVSHEDP
EVQFNWYVDG MEVHNAKTKP REEQFNSTER VVSVLTVVHQ DWLNGKEYKC
KVSNKGLPAP IEKTISKTKG QPREPQVYTL PPSREEMTKN QVSLTCLVKG
FYPSDIAVEW ESNGQPENNY KTTPPMLDSD GSFFLYSKLT VDKSRWQQGN
VFSCSVMHEA LHNHYTQKSL SLSPGK
SEQ ID NO: 16
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVTSSNFGT
QTYTCNVDHK PSNTKVDKTV ERKCCVECPP CPAPPVAGPS VFLFPPKPKD
TLYITREPEV TCVVVDVSHE DPEVQFNWYV DGMEVHNAKT KPREEQFNST
FRVVSVLTVV HQDWLNGKEY KCKVSNKGLP APIEKTISKT KGQPREPQVY
TLPPSREEMT KNQVSLTCLV KGFYPSDIAV EWESNGQPEN NYKTTPPMLD
SDGSFFLYSK LTVDKSRWQQ GNVFSCSVMH EALHNHYTQK SLSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNFGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQFNST
YRVVSVLTVL HQDWINGKEY KCKVSNKGLP SSIEKTISKA KGQPREPQVY
TLPPSQEEMT KNQVSLTCLV KGFYPSDIAV EWESNGQPEN NYKTTPPVLD
SDGSFFLYSR LTVDKSRWQE GNVFSCSVMH EALHNHYTQK SLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAE VKKPGSSVKV SCKASGGTES SYAISVWRQA PGQGLEWMGG
IGPFFGTANY AQKFQGRVTI TADESTSTAY MELSSLRSED TAVYYCARDT
PYFDYWGQGT LVTVSS
SEQ ID NO: 28
DIELTQPPSV SVAPGQTARI SCSGDSIPNY YVYWYQQKPG QAPVIVIYDD
SNRPSGIPER FSGSNSGNTA TLTISGTQAE DEADYYCQSF DSSLNAEVFG
GGTKLTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAE VKKPGESLKI SCKGSGYSFT NYISWVRQMP GKGLEWMGII
DPDDSYTEYS PSFQGQVTIS ADKSISTAYL QWSSLKASDT AMYYCARYEY
GGFDIWGQGT LVTVSS
SEQ ID NO: 36 TKLTVL
SYELTQPPSV SVAPGQTARI SCSGDNIGNS YVHWYQQKPG QAPVLVIYKD
TLTISGTQAE DEADYYCGTY DIESYVFGGG
TKLTVL
SEQ ID NO: 37
SSYYVA
SEQ ID NO: 38
AIYTGSGATYKASWAKG
SEQ ID NO: 39
DGGYDYPTHAMHY
SEQ ID NO: 40
QASQNIGSSLA
SEQ ID NO: 41
GASKTHS
SEQ ID NO: 42
QSTKVGSSYGNH
SEQ ID NO: 43
QVQLVESGGG LVQPGGSLRL SCAASGFTSH SSYYVAWVRQ APGKGLEWVG
AIYTGSGATY KASWAKGRFT ISKDTSKNQV VLTMTNMDPV DTATYYCASD
GGYDYPTHAM HYWGQGTLVT VSS
SEQ ID NO: 44
DVVMTQSPSS LSASVGDRVT ITCQASQNIG SSLAWYQQKP GQAPRLLIYG
ASKTHSGVPS RFSGSGSGTD FTLTISSLQP EDVATYYCQS TKVGSSYGNH
FGGGTKVEIK
SEQ ID NO: 45
QVQLVESGGG LVQPGRSLRL SCAASGFTVH SSYYMAWVRQ APGKGLEWVG
AIFTGSGAEY KAEWAKGRVT ISKDTSKNQV VLTMTNMDPV DTATYYCASD
AGYDYPTHAM HYWGQGTLVT VSSASTKGPS VFPLAPSSKS TSGGTAALGC
LVKDYFPEPV TVSWNSGALT SGVHTFPAVL QSSGLYSLSS VVTVPSSSLG
TQTYICNVNH KPSNTKVDKK VEPKSCDKTH TCPPCPAPEL RRGPKVFLFP
PKPKDTLMIS RTPEVTCVVV DVSHEDPEVK FNWYVDGVEV HNAKTKPREE
QYNSTYRVVS VLTVLHQDWL NGKEYKCKVS NKGLPSSIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVLHEALHA HYTRKELSLS
P
SEQ ID NO: 46
DIQMTQSPSS LSASVGDRVT ITCRASQGIS SSLAWYQQKP GKAPKLLIYG
ASETESGVPS RFSGSGSGTD FTLTISSLQP EDFATYYCQN TKVGSSYGNT
FGGGTKVEIK RTVAAPSVFI FPPSDEQLKS GTASVVCLLN NFYPREAKVQ
WKVDNALQSG NSQESVTEQD SKDSTYSLSS TLTLSKADYE KHKVYACEVT
HQGLSSPVTK SENRGEC
SEQ ID NO: 47
QVQLQESGPGLVKPSETLSLTCTVSGDSVSSSYWTWIRQPPGKGLEWIGYIYYSGSSN
YNPSLKSRATISVDTSKNQFSLKLSSVTAADTAVYYCAREGNVDTTMI FDYWGQGTLV
TVSS
SEQ ID NO: 48
AIQMTQSPSSLSASVGDRVTITCRASQGIRNDLGWY QQKPGKAPKLLIYAASSLQSGVP
SRFAGRGSGTDFTLTISSLQPEDFATYYCLQDENYPWTFGQGTKVEIK
SEQ ID NO: 49
QVQLQESGPGLVKPSETLSLTCTVSGDSVSSSYWTWIRQPPGKGLEWIGYIYYSGSSNY
NPSLKSRATISVDTSKNQFSLKLSSVTAADTAVYYCAREGNVDTTMIFDYWGQGTLVTV
SSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTVD
KSRWQEGNVFSCSVMHEALHNHYTQKSLSLSLGK
SEQ ID NO: 50
AIQMTQSPSSLSASVGDRVTITCRASQGIRNDLGWYQQKPGKAPKLLIYAASSLQSGVP
SRFAGRGSGTDFTLTISSLQPEDFATYYCLQDFNYPWTFGQGTKVEIKRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSENRGEC

1. A method of treating a human patient with hematopoietic stem cell transplant-associated thrombotic microangiopathy (HSCT-TMA), the method comprising administering to the patient an effective amount of an anti-C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof, is administered:
(a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

2. A method of treating a human patient with hematopoietic stem cell transplant-associated thrombotic microangiopathy (HSCT-TMA), the method comprising administering to the patient an effective amount of an anti-C5 antibody or antigen binding fragment thereof comprising CDR1, CDR2 and CDR3 heavy chain sequences as set forth in SEQ ID NOs:19, 18 and 3, respectively, CDR1, CDR2 and CDR3 light chain sequences as set forth in SEQ ID NOs:4, 5 and 6, respectively, and a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering, wherein the anti-C5 antibody or antigen binding fragment thereof is administered:
(a) once on Day 1 at a dose of 600 mg to a patient weighing ≥5 to <10 kg, 600 mg to a patient weighing ≥10 to <20 kg, 900 mg to a patient weighing ≥20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a dose of 300 mg to a patient weighing ≥5 to <10 kg, 300 mg to a patient weighing ≥10 to <20 kg, 300 mg to a patient weighing ≥20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and every four weeks thereafter at a dose of 300 mg to a patient weighing ≥5 to <10 kg or 600 mg to a patient weighing ≥10 to <20 kg; or on Day 15 and every eight weeks thereafter at a dose of 2100 mg to a patient weighing ≥20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

3. The method of claim 1, wherein the anti-C5 antibody comprises a heavy chain variable region set forth in SEQ ID NO:12 and a light chain variable region set forth in SEQ ID NO:8.

4. The method of claim 1, wherein the anti-C5 antibody further comprises a heavy chain constant region set forth in SEQ ID NO:13.

5. The method claim 1, wherein the antibody comprises a heavy chain polypeptide comprising the amino acid sequence set forth in SEQ ID NO:14 and a light chain polypeptide comprising the amino acid sequence set forth in SEQ ID NO:11.

6. The method of claim 1, wherein the anti-C5 antibody binds to human C5 at pH 7.4 and 25 C with an affinity dissociation constant (K_{D}) that is in the range 0.1 nM ≤K_{D}≤1 nM (e.g., about 0.5 nM).

7. The method of claim 1, wherein the anti-C5 antibody binds to human C5 at pH 6.0 and 25 C with a K_{D}≥10 nM (e.g., about 22 nM).

8. The method of claim 1, wherein the anti-C5 antibody is administered to a patient weighing ≥5 to <10 kg:
(a) once on Day 1 at a dose of 600 mg;
(b) once on Day 5 at a dose of 300 mg;
(c) once on Day 10 at a dose of 300 mg; and
(d) on Day 15 and every four weeks thereafter at a dose of 300 mg.

9. The method of claim 1, wherein the anti-C5 antibody is administered to a patient weighing ≥10<20 kg:
(a) once on Day 1 at a dose of 600 mg;
(b) once on Day 5 at a dose of 300 mg;
(c) once on Day 10 at a dose of 300 mg; and
(d) on Day 15 and every four weeks thereafter at a dose of 600 mg.

10. The method of claim 1, wherein the anti-C5 antibody is administered to a patient weighing ≥20 to <30 kg:
(a) once on Day 1 at a dose of 900 mg;
(b) once on Day 5 at a dose of 300 mg;
(c) once on Day 10 at a dose of 300 mg; and
(d) on Day 15 and every eight weeks thereafter at a dose of 2100 mg.

11. The method of claim 1, wherein the anti-C5 antibody is administered to a patient weighing ≥30 to <40 kg:
(a) once on Day 1 at a dose of 1200 mg;
(b) once on Day 5 at a dose of 300 mg;
(c) once on Day 10 at a dose of 300 mg; and
(d) on Day 15 and every eight weeks thereafter at a dose of 2700 mg.

12. The method of claim 1, wherein the anti-C5 antibody is administered to a patient weighing ≥40 to <60 kg:
(a) once on Day 1 at a dose of 2400 mg;
(b) once on Day 5 at a dose of 600 mg;
(c) once on Day 10 at a dose of 600 mg; and
(d) on Day 15 and every eight weeks thereafter at a dose of 3000 mg.

13. The method of claim 1, wherein the anti-C5 antibody is administered to a patient weighing ≥60 to <100 kg:
(a) once on Day 1 at a dose of 2700 mg;
(b) once on Day 5 at a dose of 900 mg;
(c) once on Day 10 at a dose of 900 mg; and
(d) on Day 15 and every eight weeks thereafter at a dose of 3300 mg.

14. The method of claim 1, wherein the anti-C5 antibody is administered to a patient weighing ≥100 kg:
(a) once on Day 1 at a dose of 3000 mg;
(b) once on Day 5 at a dose of 900 mg;
(c) once on Day 10 at a dose of 900 mg; and
(d) on Day 15 and every eight weeks thereafter at a dose of 3600 mg.

15. The method of claim 1, wherein the treatment maintains a serum trough concentration of the anti-C5 antibody of 100 μg/mL or greater.

16. The method of any one of claim 1, wherein the treatment maintains a serum trough concentration of the anti-C5 antibody of 200 μg/mL or greater.

17. The method of claim 1, wherein the anti-C5 antibody is formulated for intravenous administration.

18. (canceled)

19. The method of claim 1, wherein the treatment results in one or more of the following:
(1) a reduction or cessation in microangiopathic hemolytic anemia, thrombocytopenia, endothelial injury, kidney damage, kidney failure, serositis, pulmonary hypertension, and multisystem organ failure compared to baseline;
(2) platelet count ≥50,000/mm3 without transfusion support during the prior 7 days, LDH<1.5×ULN, and absence of schistocytes (if there were schistocytes present at baseline);
(3) platelet count ≥50,000/mm³ without transfusion support during the prior 7 days, LDH<1.5×ULN, absence of schistocytes (if there were schistocytes present at baseline) and at least 50% reduction of proteinuria from baseline;
(4) a favorable hematological response;
(5) normalization of LDH, resolution of need for red cell and platelet transfusions, and disappearance of schistocytes;
(6) hemoglobin ≥8 g/dL without transfusion support;
(7) a decrease in LDH, an increase in platelets, and/or an increase in hemoglobin compared to baseline;
(8) normal levels of serum creatinine compared to baseline;
(9) an improvement in TMA-associated organ dysfunction in the renal, cardiovascular, pulmonary, CNS, and/or GI systems compared to baseline;
(10) terminal complement inhibition;
(11) a reduction in adverse events;
(12) a shift toward normal levels of biomarkers associated with vascular inflammation (e.g., shed tumor necrosis factor receptor 1 [TNF-R1]), endothelial damage and/or activation (e.g., thrombomodulin and shed vascular cell adhesion molecule 1 [VCAM-1]), renal injury (e.g., Cystatin C), and/or complement proteins and complement activation pathway products;
(13) a change from baseline in quality of life as assessed via a Quality of Life Assessment;
(14) a reduction of lactate dehydrogenase (LDH) levels compared to baseline; and/or
(15) a reduction in free C5 concentration in the patient or reduction in red blood cell (RBC) hemolysis: particularly wherein the treatment results in free C5 concentration of 0.5 μg/mL or less and/or RBC hemolysis of 20% or less compared to an untreated patient.

20.-34. (canceled)

35. The method of claim 1, wherein the treatment maintains a serum trough concentration of the anti-C5 antibody, or antigen binding fragment thereof, in the patient of at least 175 μg/mL or greater.

36. The method of claim 1, wherein the human patient is a pediatric patient.

37. The method of claim 1, wherein the human patient is an adult patient.

38. The method of claim 1, further comprising administering one or more best supportive care (BSC) measures selected from the group consisting of transfusion support, corticosteroids, dialysis, and antihypertensive medications.

39. (canceled)

40. A kit for treating hematopoietic stem cell transplant-associated thrombotic microangiopathy (HSCT-TMA), in a human patient, the kit comprising:
(a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and
(b) instructions for using the anti-C5 antibody, or antigen binding fragment thereof, in the method of claim 1.

41.-42. (canceled)


SEQUENCE LISTING (50 sequences)

SEQ ID NO: 1 - protein, 10 aa, Artificial Sequence
  source: /note=“Description of Artificial Sequence Synthetic peptide”
GYIFSNYWIQ

SEQ ID NO: 2 - protein, 17 aa, Artificial Sequence
  source: /note=“Description of Artificial Sequence Synthetic peptide”
EILPGSGSTEYTENFKD

SEQ ID NO: 3 - protein, 13 aa, Artificial Sequence
  source: /note=“Description of Artificial Sequence Synthetic peptide”
YFFGSSPNWYFDV

SEQ ID NO: 4 - protein, 11 aa, Artificial Sequence
  source: /note=“Description of Artificial Sequence Synthetic peptide”
GASENIYGALN

SEQ ID NO: 5 - protein, 7 aa, Artificial Sequence
  source: /note=“Description of Artificial Sequence Synthetic peptide”
GATNLAD

SEQ ID NO: 6 - protein, 9 aa, Artificial Sequence
  source: /note=“Description of Artificial Sequence Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 447 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence Synthetic peptide”
GASENIYHALN

SEQ ID NO: 18 - protein, 17 aa, Artificial Sequence
  source: /note=“Description of Artificial Sequence Synthetic peptide”
EILPGSGHTEYTENFKD

SEQ ID NO: 19 - protein, 10 aa, Artificial Sequence
  source: /note=“Description of Artificial Sequence Synthetic peptide”
GHIFSNYWIQ

SEQ ID NO: 20 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  source: /note=“Description of Artificial Sequence Synthetic peptide”
SYAIS

SEQ ID NO: 22 - protein, 17 aa, Artificial Sequence
  source: /note=“Description of Artificial Sequence Synthetic peptide”
GIGPFFGTANYAQKFQG

SEQ ID NO: 23 - protein, 7 aa, Artificial Sequence
  source: /note=“Description of Artificial Sequence Synthetic peptide”
DTPYFDY

SEQ ID NO: 24 - protein, 11 aa, Artificial Sequence
  source: /note=“Description of Artificial Sequence Synthetic peptide”
SGDSIPNYYVY

SEQ ID NO: 25 - protein, 7 aa, Artificial Sequence
  source: /note=“Description of Artificial Sequence Synthetic peptide”
DDSNRPS

SEQ ID NO: 26 - protein, 11 aa, Artificial Sequence
  source: /note=“Description of Artificial Sequence Synthetic peptide”
QSFDSSLNAEV

SEQ ID NO: 27 - protein, 116 aa, Artificial Sequence
  source: /note=“Description of Artificial Sequence Synthetic polypeptide”
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  source: /note=“Description of Artificial Sequence Synthetic polypeptide”
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  source: /note=“Description of Artificial Sequence Synthetic peptide”
NYIS

SEQ ID NO: 30 - protein, 17 aa, Artificial Sequence
  source: /note=“Description of Artificial Sequence Synthetic peptide”
IIDPDDSYTEYSPSFQG

SEQ ID NO: 31 - protein, 8 aa, Artificial Sequence
  source: /note=“Description of Artificial Sequence Synthetic peptide”
YEYGGFDI

SEQ ID NO: 32 - protein, 11 aa, Artificial Sequence
  source: /note=“Description of Artificial Sequence Synthetic peptide”
SGDNIGNSYVH

SEQ ID NO: 33 - protein, 7 aa, Artificial Sequence
  source: /note=“Description of Artificial Sequence Synthetic peptide”
KDNDRPS

SEQ ID NO: 34 - protein, 9 aa, Artificial Sequence
  source: /note=“Description of Artificial Sequence Synthetic peptide”
GTYDIESYV

SEQ ID NO: 35 - protein, 116 aa, Artificial Sequence
  source: /note=“Description of Artificial Sequence Synthetic polypeptide”
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  source: /note=“Description of Artificial Sequence Synthetic polypeptide”
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, Artificial Sequence
  source: /note=“Description of Artificial Sequence Synthetic peptide”
SSYYVA

SEQ ID NO: 38 - protein, 17 aa, Artificial Sequence
  source: /note=“Description of Artificial Sequence Synthetic peptide”
AIYTGSGATYKASWAKG

SEQ ID NO: 39 - protein, 13 aa, Artificial Sequence
  source: /note=“Description of Artificial Sequence Synthetic peptide”
DGGYDYPTHAMHY

SEQ ID NO: 40 - protein, 11 aa, Artificial Sequence
  source: /note=“Description of Artificial Sequence Synthetic peptide”
QASQNIGSSLA

SEQ ID NO: 41 - protein, 7 aa, Artificial Sequence
  source: /note=“Description of Artificial Sequence Synthetic peptide”
GASKTHS

SEQ ID NO: 42 - protein, 12 aa, Artificial Sequence
  source: /note=“Description of Artificial Sequence Synthetic peptide”
QSTKVGSSYGNH

SEQ ID NO: 43 - protein, 123 aa, Artificial Sequence
  source: /note=“Description of Artificial Sequence Synthetic polypeptide”
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, Artificial Sequence
  source: /note=“Description of Artificial Sequence Synthetic polypeptide”
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 45 - protein, 451 aa, Artificial Sequence
  source: /note=“Description of Artificial Sequence Synthetic polypeptide”
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 217 aa, Artificial Sequence
  source: /note=“Description of Artificial Sequence Synthetic polypeptide”
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 120 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 48 - protein, 107 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 49 - protein, 447 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC